SEQUENCE LISTING (19 sequences)

SEQ ID NO: 1 - RNA, 19 nt, Artificial
  siRNA
uagcuuuauauggauguua

SEQ ID NO: 2 - RNA, 19 nt, Artificial
  siRNA
uaaaacccuguaaggaaaa

SEQ ID NO: 3 - RNA, 19 nt, Artificial
  siRNA
ucuaaaacccuguaaggaa

SEQ ID NO: 4 - RNA, 19 nt, Artificial
  siRNA
gauuuugucuaaaacccug

SEQ ID NO: 5 - RNA, 19 nt, Artificial
  siRNA
uuugauuuugucuaaaacc

SEQ ID NO: 6 - RNA, 19 nt, Artificial
  siRNA
cuuucauaaugcuggcaga

SEQ ID NO: 7 - RNA, 19 nt, Artificial
  siRNA
ucacuuucauaaugcuggc

SEQ ID NO: 8 - RNA, 19 nt, Artificial
  siRNA
cuuucuaacaucugaacuu

SEQ ID NO: 9 - RNA, 19 nt, Artificial
  siRNA
caacuuucuaacaucugaa

SEQ ID NO: 10 - RNA, 19 nt, Artificial
  siRNA
uagcuuuauuuggauguua

SEQ ID NO: 11 - RNA, 19 nt, Artificial
  siRNA
guuucacaacacauuuuac

SEQ ID NO: 12 - RNA, 19 nt, Artificial
  siRNA
uuuugucuaauacccugua

SEQ ID NO: 13 - RNA, 19 nt, Artificial
  siRNA
gauuuugucaaaaacccug

SEQ ID NO: 14 - RNA, 19 nt, Artificial
  siRNA
uuugauuuugacuaaaacc

SEQ ID NO: 15 - RNA, 19 nt, Artificial
  siRNA
aggaaugugaccaccuucc

SEQ ID NO: 16 - RNA, 19 nt, Artificial
  siRNA
cuuucauaaagcuggcaga

SEQ ID NO: 17 - RNA, 19 nt, Artificial
  siRNA
ucacuuucaaaaugcuggc

SEQ ID NO: 18 - RNA, 19 nt, Artificial
  siRNA
cuuucuaacuucugaacuu

SEQ ID NO: 19 - RNA, 19 nt, Artificial
  siRNA
gcuauaccagcgucgucau

(19) United States
(12) Patent Application Publication
(10) Pub. No.: US 2015/0164901 A1
(43) Pub. Date: Jun. 18, 2015

(54) COMPOUNDS, COMPOSITIONS AND METHODS FOR TREATING OR PREVENTING NEURODEGENERATIVE DISORDERS

(71) Applicant: President and Fellows of Harvard College, Cambridge, MA (US)

(72) Inventor: Lee L. Rubin, Wellesley, MA (US)

(21) Appl. No.: 14/571,005

(22) Filed: Dec. 15, 2014

Related U.S. Application Data
(63) Continuation-in-part of application No. PCT/US2013/046172, filed on Jun. 17, 2013.
(60) Provisional application No. 61/660,396, filed on Jun. 15, 2012.
(60) Provisional application No. 61/767,002, filed on Feb. 20, 2013.
(60) Provisional application No. 61/767,404, filed on Feb. 21, 2013.

(51) Int. Cl. A61K 31/519; A61K 45/06

(52) U.S. Cl. CPC A61K 31/519; A61K 45/06

(57) ABSTRACT

The disclosure provides compounds, compositions, and methods for promoting motor neuron survival, and for treating or preventing neurodegenerative disorders, such as spinal muscular atrophy (SMA) and amyotrophic lateral sclerosis (ALS).

### RELATED APPLICATIONS

This application is a continuation-in-part of PCT Application No. PCT/US2013/046172, filed Jun. 17, 2013, which claims the benefit of U.S. Provisional Application No. 61/660,396, filed Jun. 15, 2012, U.S. Provisional Application No. 61/767,002, filed Feb. 20, 2013, and U.S. Provisional Application No. 61/767,404, filed Feb. 21, 2013. The entire teachings of the above applications are incorporated herein by reference.

### GOVERNMENT SUPPORT

This invention was made with government support under NS066888 awarded by the National Institutes of Health. The government has certain rights in the invention.

### BACKGROUND OF THE INVENTION

Spinal Muscular Atrophy (SMA) is the leading genetic cause of death in infants. This neurodegenerative disease results from diminished levels of the protein Survival of Motor Neuron (SMN) with motor neurons being the cell type most affected, although recent evidence suggests that other cells, such as muscle, might contribute to the disease phenotype. Data derived from SMA patients and from SMA mouse models suggest that therapeutics that elevate Survival of Motor Neuron (SMN) levels will be effective in treating this disease. Current treatment for SMA consists of prevention and management of the secondary effect of chronic motor unit loss. Some drugs under clinical investigation for treatment of SMA include, e.g., Bbutyrates, Valproic acid, Hydroxyurea and Riluzole. However, there are currently no known treatments for SMA that elevate SMN levels.

Therefore, there is need in the art for compositions and methods for treatment of SMA and other neurodegenerative disorders involving diminished levels of the protein SMN, such as Amyotrophic Lateral Sclerosis (ALS).

### SUMMARY OF THE INVENTION

In accordance with an embodiment of the present invention, a method of regulating survival of motor neuron (SMN) protein levels in a cell is provided. The method includes contacting the cell with an effective amount of an agent which inhibits the level or activity of Nedd8 activating enzyme (NAE) in the cell, thereby regulating SMN protein levels in the cell.

In accordance with an embodiment of the present invention, a method of promoting cell survival in a subject in need thereof by regulating the overall levels of SMN protein in the subject is provided. The method includes administering to a subject an agent which inhibits the level or activity of Nedd8 activating enzyme (NAE) in the subject's cells, thereby promoting cell survival in the subject.

In accordance with an embodiment of the present invention, a method of treating or preventing a neurodegenerative disorder in a subject in need thereof is provided. The method includes administering to the subject at least one agent which inhibits the level or activity of Nedd8 activating enzyme (NAE).

In accordance wish an embodiment of the present invention, a method of treating or preventing a neurodegenerative disorder in a subject in need thereof is provided. The method includes (a) obtaining a biological sample comprising cells from a subject suspected of having a neurodegenerative disorder; (b) contacting the cells with an effective amount of at least one agent which inhibits the level or activity of Nedd8 activating enzyme (NAE) in the cells, and (c) administering the cells to the subject, thereby treating or preventing the neurodegenerative disorder.

In accordance with aspects of the present invention, regulating SMN protein levels in the cell is selected from the group consisting of (i) increasing the levels of SMN protein in the cell, (ii) stabilizing levels of SMN protein in the cell, (iii) reducing degradation of SMN protein in the cell, (iv) preventing degradation of SMN protein in the cell, (v) modulating splicing of SMN mRNA to increase the levels of full-length SMN protein, and (vi) any combination of (i)-(v).

In accordance with aspects of the present invention, regulating the overall levels of SMN protein in the subject is selected from the group consisting of: (i) increasing the levels of SMN protein in the subject's cells, (ii) stabilizing levels of SMN protein in the subject's cells, (iii) reducing degradation of SMN protein in the subject's cells, (iv) preventing degradation of SMN protein in the subject's cells, (v) modulating splicing of SMN mRNA to increase the levels of full-length SMN protein, (vi) increasing the number of high SMN expressing cells in the subject (vii) stabilizing the number of high SMN expressing cells in the subject, (viii) increasing the ratio of high SMN expressing cells to low SMN expressing cells in the subject, and(ix) decreasing the number of low SMN expressing cells in the subject, and (x) any combination of (i)-(ix).

In accordance with aspects of the present invention, the agent is a compound of formula (I):

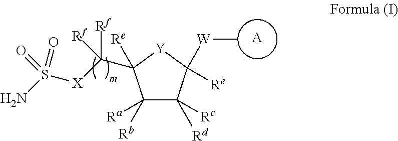

wherein:
Ring A is a 6-membered nitrogen-containing heteroaryl ring, optionally fused to a 5- or 6-membered aryl, heteroaryl, cycloaliphatic or heterocyclic ring, wherein either or both rings is optionally substituted and one ring nitrogen atom is optionally oxidized or Ring A is selected from the group consisting of

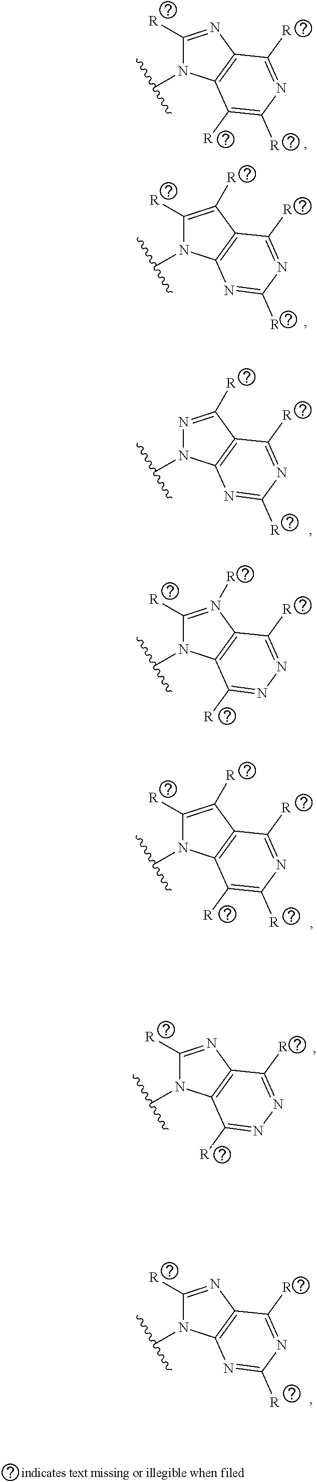

### W is CH₂, CHF, CH₂, CH(R1), CH(R¹), NH, N(R), O, S, or —NHC(O)—;

R¹ is C₁₋₄ aliphatic or C₂₋₄ fluoroaliphatic; or R¹ is a C₂₋₄ alkylene chain that is attached to a ring position on Ring A to form a 5-, 6-, or 7-membered fused ring, wherein the alkylene chain optionally is substituted with C₁₋₄ aliphatic, C₁₋₄ fluoroaliphatic, —O, —CN, or —C(O)N(R⁴)2;

### X is —O—, —S—, or —C(R^{m})(Rⁿ)—;

R^{a} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)NR⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;
R^{b} is selected from the group consisting of hydrogen, fluoro, C₁₋₄ aliphatic, and C₁₋₄ fluoroaliphatic;
R^{c} is selected from the group consisting of hydrogen, fluoro, CN, N₄, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;
R^{d} is selected from the group consisting of hydrogen, fluoro, C₁₋₄ aliphatic, and C₁₋₄ fluoroaliphatic;
R^{e} is hydrogen, or C₁₋₄ aliphatic; or R^{e}, taken together with one R^{f} and the intervening carbon atoms, forms a 3- to 6-membered spirocyclic ring; or R^{e}, taken together with R^{m} and the intervening carbon atoms, forms a fused cyclopropane ring, which is optionally substituted with one or two substituents independently selected from fluoro or C₁₋₄ aliphatic;
R^{e′} is hydrogen or C₁₋₄ aliphatic; or R^{e′}, taken together with R^{m} and the intervening carbon atoms, forms a fused cyclopropane ring, which is optionally substituted with one or two substituents independently selected from fluoro or C₁₋₄ aliphatic;
each R^{f} is independently hydrogen, fluoro, C₁₋₄ aliphatic, or C₁₋₄ fluoroaliphatic; or two R^{f} taken together form ═O; or two R^{f}, taken together with the carbon atom to which they are attached, form a 3- to 6-membered carbocyclic ring; or one R^{f}, taken together with R^{e} and the intervening carbon atoms, forms a 3- to 6-membered spirocyclic ring;
R^{m} is hydrogen, fluoro, —N(R⁴)₂, or an optionally substituted C₁₋₄ aliphatic group; or R^{m} and R^{a} together form ═O or ═C(R⁵)₂; or R^{m} and R^{e}, taken together with the intervening carbon atoms, form a fused cyclopropane ring, which is optionally substituted with one or two substituents independently selected from fluoro or C₁₋₄ aliphatic; or R^{m} and R^{e′}, taken together with the intervening carbon atoms, form a fused cyclopropane ring, which is optionally substituted with one or two substituents independently selected from fluoro or C₁₋₄ aliphatic;
Rⁿ is hydrogen, fluoro, or an optionally substituted C₁₋₄ aliphatic group; or R^{m} and Rⁿ together form ═O or ═C(R⁵)₂;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, in addition to the nitrogen atom, 0-2 ring heteroatoms independently selected from N, O, and S;
R^{4x} is hydrogen, C₁₋₄ alkyl, C₁₋₄ fluoroalkyl, or C₆₋₁₀ar(C₁₋₄)alkyl, the aryl portion of which can be optionally substituted;
R^{4y} is hydrogen, C₁₋₄ alkyl, C₁₋₄ fluoroalkyl, C₆₋₁₀ar(C₁₋₄)alkyl, the aryl portion of which can be optionally substituted, or an optionally substituted 5- or 6-membered aryl, heteroaryl, or heterocyclyl ring; or
R^{4x} and R^{4y}, taken together with the nitrogen atom to which they are attached, form an optionally substituted 4- to 5-membered heterocyclyl ring having, in addition to the nitrogen atom, 0-2 ring heteroatoms independently selected from N, O, and S; and
each R⁵ independently is hydrogen or an optionally substituted aliphatic, aryl, heteroaryl, or heterocyclyl group;
each R^{5x} independently is hydrogen, C₁₋₄ alkyl, C₁₋₄ fluoroalkyl, or an optionally substituted C₆₋₁₀ aryl or C₆₋₁₀ar(C₁₋₄)alkyl;
each R⁶ independently is an optionally substituted aliphatic, aryl or heteroaryl group; R^{g} is hydrogen, halo, —NO₂, —CN, —C(R⁵)═C(R⁵)₂, —C═C—R, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OCO₂R⁶, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)₂, —N(R⁴)—N(R⁴)—C(O)R⁵, —C(═NR⁴)—N(R⁴)₂, —C(═NR⁴)—OR⁵, —N(R⁴)—N(R⁴)₂, —N(R⁴)—OR⁵, —C(═NR⁴)—N(R⁴)—OR⁵, —C(R⁶)═N—OR⁵, or an optionally substituted aliphatic, aryl, heteroaryl, or heterocyclyl;
each R^{h} independently is hydrogen, halo, —CN—, —OR⁵, —N(R⁴)², —SR⁶, or an optionally substituted C1-4 aliphatic group;
each R^{j} is hydrogen, —OR⁵, —SR⁶, —N(R⁴)₂, or an optionally substituted aliphatic, aryl or heteroaryl group;
R^{k} is hydrogen, halo, —OR⁵, —SR⁶, —N(R⁴)+, or an optionally substituted C₁₋₄ aliphatic group;
m is 1, 2, or 3;
and pharmaceutically acceptable salts thereof.

In accordance with aspects of the present invention, the agent comprises the compound ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate (MLN4924).

In accordance with aspects of the present invention, the agent comprises the compound ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate (MLN4924).

In accordance with aspects of the present invention, the method further includes contacting the cell with an effective amount of an agent which increases the level of SMN protein selected from the group consisting of a PI3 kinase inhibitor, an RTK ligand, GSK3 kinase inhibitors, and a splicing modulator. In accordance with aspects of the present invention, the cell comprises a neuron selected from the group consisting of cortical neurons, dopaminergic neurons, and motor neurons.

In accordance with aspects of the present invention, contacting the cell occurs ex vivo or in vitro and the cell is obtained from a human or animal subject selected for treatment of a neurodegenerative disorder selected from the group consisting of: (i) a neurodegenerative disorder characterized by degeneration of motor neurons; (ii) a neurodegenerative disorder characterized by diminished levels of SMN protein; (iii) a neurodegenerative disorder characterized by degradation of SMN protein; or iv) a neurodegenerative disorder is selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telanglectasia; Batten disease; Canavan disease; Cockayne syndrome; corticobasal degeneration; Creutzfeldt-Jakob disease; ischemia stroke; Krabbe disease, Lewy body dementia; multiple sclerosis; multiple system atrophy; Parkinson's disease; Pelizaeus-Merzbacher disease; peripheral neuropathy; Pick's disease; primary lateral sclerosis; Refsum's disease; Sandhoff disease; Schilder's disease; spinal cord injury; spinal muscular atrophy (SMA); Steele Richardson-Olszewski disease; Tabes dorsalis; and traumatic brain injury.

In accordance with aspects of the present invention, contacting the cell occurs in vivo in a subject selected for treatment of a neurodegenerative disorder is selected from the group consisting of: (i) a neurodegenerative disorder characterized by degeneration of motor neurons; (ii) a neurodegenerative disorder characterized by diminished levels of SMN protein; (iii) a neurodegenerative disorder characterized by degradation of SMN protein, or iv) a neurodegenerative disorder selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telangiectasia; Batten disease; Canavan disease; Cockayne syndrome; corticobasal degeneration; Creutzfeldt-Jakob disease; ischemia stroke; Krabbe disease; Lewy body dementia; multiple sclerosis; multiple system atrophy; Parkinson's disease; Pelizaeus-Merzbacher disease; peripheral neuropathy; Pick's disease; primary lateral sclerosis; Refsum's disease; Sandhoff disease; Schilder's disease; spinal cord injury; spinal muscular atrophy (SMA); Steele Richardson-Olszewski disease; Tabes dorsalis; and traumatic brain injury.

In accordance with aspects of the present invention, the method further includes administering to the subject an effective amount of an agent which increases the level of SMN protein selected from the group consisting of a PI3 kinase inhibitor, an RTK ligand, GSK3 kinase inhibitors, and a splicing modulator. In accordance with aspects of the present invention, the subject is selected for treatment of a neurodegenerative disorder selected from the group consisting of: (i) a neurodegenerative disorder characterized by degeneration of motor neurons; (ii) a neurodegenerative disorder characterized by diminished levels of SMN protein; (iii) neurodegenerative disorder is characterized by degradation of SMN protein; or (iv) a neurodegenerative disorder selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telangiectasia; Batten disease; Canavan disease; Cockayne syndrome; corticobasal degeneration; Creutzfeldt-Jakob disease; ischemia stroke; Krabbe disease; Lewy body muscular atrophy (SMA); Steele Richardson-Olszewski disease; Tabes dorsalis; and traumatic brain injury.

In accordance with aspects of the present invention, the at least one agent is a compound of formula (I):

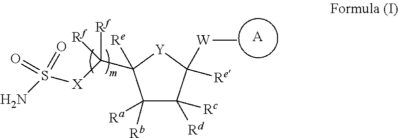

wherein:
Ring A is a 6-membered nitrogen-containing heteroaryl ring, optionally fused to a 5- or 6-membered aryl, heteroaryl, cycloaliphatic or heterocyclic ring, wherein either or both rings is optionally substituted and one ring nitrogen atom is optionally oxidized or Ring A is selected from the group consisting of

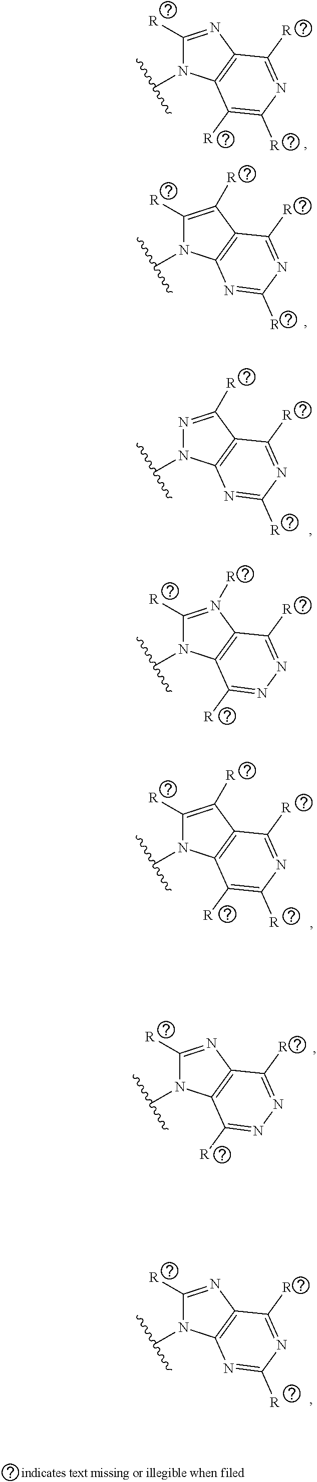

### W is CH₂, CHF, CH₂, CH(R1), CH(R¹), NH, N(R), O, S, or —NHC(O)—;

R¹ is C₁₋₄ aliphatic or C₂₋₄ fluoroaliphatic; or R¹ is a C₂₋₄ alkylene chain that is attached to a ring position on Ring A to form a 5-, 6-, or 7-membered fused ring, wherein the alkylene chain optionally is substituted with C₁₋₄ aliphatic, C₁₋₄ fluoroaliphatic, —O, —CN, or —C(O)N(R⁴)2;

### X is —O—, —S—, or —C(R^{m})(R^{m})—;

### Y is —O, —S—, or —C(R^{m})(Rⁿ)—;

R^{a} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)NR⁴)₂, —OC(O)R⁵, —OCO₂R⁶, or a C₁₋₄ aliphatic or C₁₋₄ flouroaliphatic optionally substituted with one or two substituents independently selected from the group consisting of —OR^{5x}, or —N(R^{4x})(R^{6y}), —CO₂R^{5x}, or —C(O)N(R^{4x})(R4^{4y}); or R^{a} and R^{e} together form a bond;
R^{b} is selected from the group consisting of hydrogen, fluoro, C₁₋₄ aliphatic, and C₁₋₄ fluoroaliphatic;
R^{c} is selected from the group consisting of hydrogen, fluoro, CN, N₄, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;
R^{d} is selected from the group consisting of hydrogen, fluoro, C₁₋₄ aliphatic, and C₁₋₄ fluoroaliphatic;
R^{e} is hydrogen, or C₁₋₄ aliphatic; or R^{e}, taken together with one R^{f} and the intervening carbon atoms, forms a 3- to 6-membered spirocyclic ring; or R^{e}, taken together with R^{m} and the intervening carbon atoms, forms a fused cyclopropane ring, which is optionally substituted with one or two substituents independently selected from fluoro or C₁₋₄ aliphatic;
R^{e′} is hydrogen or C₁₋₄ aliphatic; or R^{e′}, taken together with R^{m} and the intervening carbon atoms, forms a fused cyclopropane ring, which is optionally substituted with one or two substituents independently selected from fluoro or C₁₋₄ aliphatic;
each R^{f} is independently hydrogen, fluoro, C₁₋₄ aliphatic, or C₁₋₄ fluoroaliphatic; or two R^{f} taken together form ═O; or two R^{f}, taken together with the carbon atom to which they are attached, form a 3- to 6-membered carbocyclic ring; or one R^{f}, taken together with R^{e} and the intervening carbon atoms, forms a 3- to 6-membered spirocyclic ring;
R^{m} is hydrogen, fluoro, —N(R⁴)₂, or an optionally substituted C₁₋₄ aliphatic group; or R^{m} and R^{a} together form ═O or ═C(R⁵)₂; or R^{m} and R^{e}, taken together with the intervening carbon atoms, form a fused cyclopropane ring, which is optionally substituted with one or two substituents independently selected from fluoro or C₁₋₄ aliphatic; or R^{m} and R^{e′}, taken together with the intervening carbon atoms, form a fused cyclopropane ring, which is optionally substituted with one or two substituents independently selected from fluoro or C₁₋₄ aliphatic;
Rⁿ is hydrogen, fluoro, or an optionally substituted C₁₋₄ aliphatic group; or R^{m} and Rⁿ together form ═O or ═C(R⁵)₂;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, in addition to the nitrogen atom, 0-2 ring heteroatoms independently selected from N, O, and S;
R^{4x} is hydrogen, C₁₋₄ alkyl, C₁₋₄ fluoroalkyl, or C₆₋₁₀ar(C₁₋₄)alkyl, the aryl portion of which can be optionally substituted;
R^{4y} is hydrogen, C₁₋₄ alkyl, C₁₋₄ fluoroalkyl, C₆₋₁₀ar(C₁₋₄)alkyl, the aryl portion of which can be optionally substituted, or an optionally substituted 5- or 6-membered aryl, heteroaryl, or heterocyclyl ring; or
R^{4x} and R^{4y}, taken together with the nitrogen atom to which they are attached, form an optionally substituted 4- to 5-membered heterocyclyl ring having, in addition to the nitrogen atom, 0-2 ring heteroatoms independently selected from N, O, and S; and
each R⁵ independently is hydrogen or an optionally substituted aliphatic, aryl, heteroaryl, or heterocyclyl group;
each R^{5x} independently is hydrogen, C₁₋₄ alkyl, C₁₋₄ fluoroalkyl, or an optionally substituted C₆₋₁₀ aryl or C₆₋₁₀ar(C₁₋₄)alkyl;
each R⁶ independently is an optionally substituted aliphatic, aryl or heteroaryl group; R^{g} is hydrogen, halo, —NO₂, —CN, —C(R⁵)═C(R⁵)₂, —C═C—R, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OCO₂R⁶, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)₂, —N(R⁴)—N(R⁴)—C(O)R⁵, —C(═NR⁴)—N(R⁴)₂, —C(═NR⁴)—OR⁵, —N(R⁴)—N(R⁴)₂, —N(R⁴)—OR⁵, —C(═NR⁴)—N(R⁴)—OR⁵, —C(R⁶)═N—OR⁵, or an optionally substituted aliphatic, aryl, heteroaryl, or heterocyclyl;
each R^{h} independently is hydrogen, halo, —CN—, —OR⁵, —N(R⁴)², —SR⁶, or an optionally substituted C1-4 aliphatic group;
each R^{j} is hydrogen, —OR⁵, —SR⁶, —N(R⁴)₂, or an optionally substituted aliphatic, aryl or heteroaryl group;
R^{k} is hydrogen, halo, —OR⁵, —SR⁶, —N(R⁴)+, or an optionally substituted C₁₋₄ aliphatic group;
m is 1, 2, or 3;
and pharmaceutically acceptable salts thereof.

In accordance with aspects of the present invention, the at least one agent comprises the compound ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate (MLN4924).

In accordance with aspects of the present invention, the neurodegenerative disorder is selected from the group consisting of: (i) a neurodegenerative disorder characterized by degeneration of motor neurons; (ii) a neurodegenerative disorder characterized by diminished levels of SMN protein; (iii) a neurodegenerative disorder is characterized by degradation of SMN protein; or a neurodegenerative disorder selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telanglectasia; Batten disease; Canavan disease; Cockayne syndrome; corticobasal degeneration; Creutzfeldt-Jakob disease; ischemia stroke; Krabbe disease, Lewy body dementia; multiple sclerosis; multiple system atrophy; Parkinson's disease; Pelizaeus-Merzbacher disease; peripheral neuropathy; Pick's disease; primary lateral sclerosis; Refsum's disease; Sandhoff disease; Schilder's disease; spinal cord injury; spinal muscular atrophy (SMA); Steele Richardson-Olszewski disease; Tabes dorsalis; and traumatic brain injury.

In accordance with aspects of the present invention, the cells are selected from the group consisting of: (i) neurons selected from the group consisting of cortical neurons and dopaminergic neurons; (ii) motor neurons; (iii) non-neuronal cells; (iv) somatic cells; and (v) fibroblasts. In accordance with aspects of the present invention, the cells are reprogrammed to induced pluripotent stem cells. In accordance with aspects of the present invention, the cells are reprogrammed to neurons. In accordance with aspects of the present invention, the cells are reprogrammed to motor neurons. In accordance with aspects of the present invention, the cells are expanded prior to administering the cells to the subject.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a diagrammatic illustration of a pathway for Cullin-mediated degradation of SMN protein.

FIGS. 2A, 2B, 2C, 2D and 2E demonstrate that Cullin-based E3 ubiquitin ligase regulates SMN protein levels. FIG. 2A is a Western blot analysis showing the levels of SMN in cells expressing wild-type SMN and the dominant negative form of Cullin1 protein. SMN is stabilized by inhibition of the Cullin1 ubiquitin ligase. FIG. 2B is a bar graph quantifying the Western blot results shown in FIG. 2A. The error bars in FIG. 2B indicate ±S.E.M. FIG. 2C shows images of cells that were fixed and stained with an SMN-specific antibody, demonstrating that MLN4924, a chemical inhibitor of Collin ligases, leads to the stabilization of SMN protein in human fibroblasts derived from SMA patients and their parents. FIGS. 2D and 2E are dose response curves of nuclear (FIG. 2D) and cytoplasmic (FIG. 2E) SMN levels in SMA patient and parent fibroblasts treated with MLN4924 at the indicated time points. The error bars in FIGS. 2D and 2B indicate ±S.E.M. SMN levels are normalized to DMSO control for each cell line at each time point.

FIGS. 3A and 3B demonstrate that cullin inhibition increases the average levels of SMN protein in motor neurons. FIG. 3A is a line graph illustrating the fold change in SMN protein levels in motor neurons treated with varying concentrations of a Cullin inhibitor compared to DMSO control. FIG. 3B shows images of immunocytochemical analysis of motor neurons which were treated with increasing amounts of a Collin inhibitor or DMSO control.

FIG. 4 is a line graph showing the percentage of motor neuron survival after treatment with a cullin inhibitor or DMSO control at the indicated time points, demonstrating that cullin inhibition promotes the survival of motor neurons.

FIGS. 5A, 5B, 5C and 5D are diagrammatic illustrations of the Cullin family of E3 ubiquitin ligases, including Cullin 1 (Cul1, FIG. 5A), Cullin 2 (Cul2, FIG. 5B), Cullin3 (Cul3, FIG. 5C) and Cullin4A (Cul4A, FIG. 5D).

FIGS. 6A, 6B, 6C, 6D, 6E, 6F and 6G demonstrate that MLN4924 increases SMN levels in motor neurons. FIG. 6A shows Western blot analysis of SMN levels assessed in lysates of motor neuron cultures treated with MLN4924 or DMSO as control for 3 days. FIGS. 6B and 6C are bar graphs quantifying the results in FIG. 6A. FIG. 6B reveals that SMN levels were significantly increased by MLN4924 treatment. FIG. 6C shows quantitative RT-PCR analysis revealing that SMN transcript levels were not significantly affected by treatment with MLN4924 compared to control. These results indicate that MLN4924 affects SMN protein stability and not transcription or splicing. FIG. 6D shows images of immunocytochemical analysis of motor neurons derived from mouse embryonic stem cells which were treated with increasing amounts of MLN4924 or DMSO control at plating and wore fixed four days later. SMN is shown in red and GFP driven by the motor neuron specific promoter hb9 is shown in green. FIG. 6E is a bar graph illustrating that the average intensify of SMN significantly increases in a dose-dependent manner with increasing levels of MLN4924. FIG. 6F is line graph showing the results of a histogram analysis of the number of motor neurosis with different SMN levels indicating that MLN4924 treatment increases the number of motor neurons with higher levels of SMN expression without affecting the amount of low SMN oppressors. FIG. 6G is a bar graph quantification which reveals a dose-dependent effect of MLN4924 on the number of high SMN expressing motor neurons. High SMN expressor is defined as those motor neurons that harbor SMN levels greater than the top 10^{th} percentile as defined in the DMSO control. Error bars indicate ±S.E.M.

FIGS. 7A, 7B and 7C demonstrate that MLN4924 promotes motor neuron survival. FIG. 7A is a line graph showing the results of a time-course analysis of motor neurons derived from ES cells expressing GFP under the motor neuron specific hb9 promoter performed with imaging every 6 hours for a 7 day period. FIG. 7A demonstrates that MLN4924 significantly increases motor neuron survival. The dashed lines represent S.E.M. FIG. 3B shows representative images of motor neuron survival experiments performed in FIG. 7A. FIG. 7C is a bar graph demonstrating that MLN4924 treatment leads to a dose-dependent significant increase in motor neuron survival in mouse-ES derived motor neurons. The error bars indicate ±S.E.M.

FIGS. 8A, 8B and 8C demonstrate that MLN4924 prevents SMN knockdown induced motor neuron death. FIG. 8A shows images of stains illustrating that knockdown of SMN using shRNA encoded in a lentivirus also expressing GFP in hb9_RFP motor neurons leads to a significant decrease in motor neuron survival. MLN4924 is able to reverse the effects of SMN knockdown on motor neuron death. Representative images are shown. FIG. 8B is a line graph quantification of the results shown in FIG. 8A, which reveals due MLN4924 significantly inhibits the effect of SMN RNAi on inducing motor neuron death. The error bars indicate a ±S.E.M. FIG. 8C is a graph showing the results of a histogram analysis revealing that SMN RNAi reduces the number of high SMN expressing motor neurons but does not substantially increase the amount of low SMN expressors. This suggests that motor neurons with extremely low levels of SMN do not survive. MLN4924 increases the number of high SMN expressing motor neurons, suggesting that this is the mechanism by which MLN4924 promotes motor neuron survival.

FIGS. 9A, 9B, 9C, and 9D demonstrate that MLN4924 increases SMN protein levels and promotes motor neuron survival in SMA-like mouse motor neurons. FIG. 9A are images of immunostains of motor neurons derived from the ES-cells of A2 mice that lack a functional copy of mSMN but are supplemented with two copies of hSMN2 and thus are deficient but not completely lacking SMN protein were treated with MLN4924 for four days, SMN is shown in red and the hb9 promoter driven-GFP is shown in green. FIGS. 9B, 9C and 9D are bar graphs qualifying the results shown in FIG. 9A, which reveal that MLN4924 significantly increases the number of surviving motor neurons (FIG. 9B), SMN levels (FIG. 9C), and the number of high SMN expressing motor neurons (FIG. 9D) in SMN-deficient motor neurons as in wild-type. The error bars indicate ±S.E.M.

FIGS. 10A, 10B, 10C, 10D, 10E and 10F demonstrate that MLN4924 promotes motor neuron survival and SMN levels in human motor neurons derived from SMA patient iPS cells. FIG. 10A are images of immunostains of motor neurons derived from human SMA Type II patient (1-51) and SMA Type I iPS cells were plated on glia in conditions that do not support progenitor cell division due to the addition of Ara C and treated with MLN4924 for a duration of 7 days. Representative images are shown for Type II motor neurons. SMN is shown in red, the motor neuron marker islet 1 is shown in green, and the nuclear marker Hoechst is shown in blue. Quantification of human motor neuron treated as in FIG. 10A reveals that MLN4924 significantly increases SMN levels (FIG. 10B), the number of surviving motor neurons (FIGS. 10C and 10E), and the number of high SMN expressing motor neurons (FIGS. 10D and 10F) in SMN-deficient human motor neurons. Error bars indicate ±S.E.M.

FIGS. 11A and 11B demonstrate that MLN4924 promotes motor neuron survival of human motor neurons derived from ALS patient iPS cells. FIG. 11A are immune stains of motor neurons derived from human wild-type (HUES3, 18a) and ALS patient (47a) ES or iPS cells. These cells were plated on glia in conditions that do not support progenitor cells, treated with MLN4924 for 14 days after 7 days in culture, and motor neuron survival was assessed. Representative images are shown for the Hues3 line which expresses GFP under the hb9 promoter shown is green, human nuclear antigen (hNA) shown in pink, and the nuclear marker Hoechst is shown in blue. FIG. 11B is a bar graph showing quantification of the results in FIG. 11A, which reveals that MLN4924 significantly increases the number of surviving motor neurons, suggesting that MLN4924 may act to preserve neuronal health and prevent neuron death in contexts outside of SMA and may be used to treat ALS and other neurodegenerative diseases. Error bars indicate ±S.E.M.

FIG. 12 is a diagrammatic illustration depicting the mechanism of action of MLN4924.

FIG. 13 is a bar graph illustrating the effect of MLN4924 treatment on Brn2+ neurons compared to DMSO control, demonstrating that MLN4924 increases survival of other neurons.

FIG. 14 is a line graph comparing the effects of Trichostatin A treatment on human motor neurons compared to a control, demonstrating that trichostatin A selectively reduces the number of low SMN expressing motor neurons.

FIG. 15 is a bar graph showing the additive effects on SMN levels of MLN and Roche small molecule SMN splicing modulators (R07, R08) when tested on a spinal muscular atrophy (SMA) induced pluripotent stem (iPS) patient cell line.

FIG. 16A, FIG. 16B, FIG. 16C, and FIG. 16D are line graphs showing that the MLN compound increases SMN levels in wilde type motor neurons (BJ RIPS (WT)), as well as three different amyotrophic lateral sclerosis (ALS) patient motor neurons (derived from iPS cells) (A19 (C9 ORF), 27B (SOD1), 1-51N (SMA type II)), whereas the splicing modulator does not.

FIG. 17 is a bar graph showing that MLN increases the survival of the indicated motor neurons (BJ RIPS (WT), 18A (WT), 27B (SOD1), 31D (TDP43), and A19 (C9ORF)), but the SMN splicing modulator does not.

FIG. 18A, FIG. 18B, FIG. 18C, and FIG. 18D are line graphs showing that MLN increases Gem counts (nuclear protein complexes important in RNA splicing and an indication of more functional SMN) in the motor neurons indicated (18A (WT), A19 (C9 ORF), 31D (TDP43), and 27B (SOD1)), and the SMN splicing modulator does not.

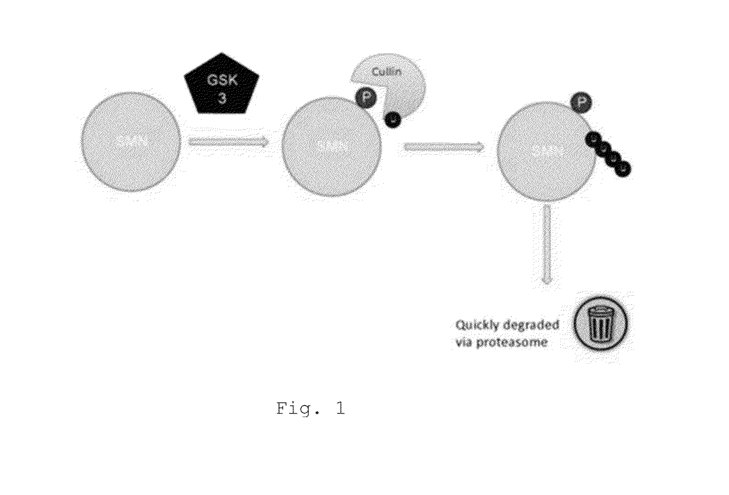

### DETAILED DESCRIPTION OF THE INVENTION

The present invention relates to methods, compositions, agents, compounds and kits for regulating the overall levels of SMN protein in cells, and use of those methods, compositions, agents, compounds and kits for promoting survival of cells (e.g., neurons, e.g., motor neurons), and for treating or preventing disorders characterized by degradation of SMN protein or diminished levels of SMN protein, such as neurodegenerative disorders (e.g., ALS or SMA).

The present invention is based, in part, on the characterization of the SMN regulatory pathway. The understanding of this pathway has revealed agents which reduce SMN protein degradation and/or increase SMN protein level, for example, by inhibiting cullin mediated degradation of SMN via ubiquitination of SMN protein. Such agents are useful in the treatment of neurodegenerative disorders, such as SMA or ALS.

Cullins are a family of hydrophobic proteins that act as scaffolds for ubiquitin ligases (E3). Cullins are found throughout eukaryotes. Humans express seven cullins (Cul1, 2, 3, 4A, 4B, 5 and 7), each forming part of a multi-subunit ubiquitin complex. Cullin-RING ubiqiutin ligases (CRLs), such as Cul1 (SCF), play an essential role in targeting proteins for ubiquitin-mediated destruction; as such, they are diverse in terms of composition and function, regulating many different processes from glucose sensing and DMA replication to limb patterning and circadian rhythms. The catalytic core of CRLs consists of a RING protein and a cullin family member. For Cul1, the C-terminal cullin-homology domain binds the RING protein. The RING protein appears to function as a docking site for ubiquitin-conjugating enzymes (E2s). Other proteins contain a cullin-homology domain, such as the APC2 subunit of the anaphase-promoting complex/cyclosome and the p53 cytoplasmic anchor PARC; both APC2 and PARC have ubiquitin ligase activity. The N-terminal region of cullins is more variable, and is used to interact with specific adaptor proteins.

With the exception of APC2, each member of the cullin family is modified by Nedd8 and several cullins function in Ubiquitin-dependent proteolysis, a process in which the 26S proteasome recognizes and subsequently degrades a target protein tagged with K48-linked poly-ubiquitin chains. Nedd8/Rub1 is a small ubiquitin-like protein, which was originally found to be conjugated to Cdc53, a cullin component of the SCF (Skp1-Cdc53/CUL1-F-box protein) E3 Ub ligase complex in Sacharomyces cerevisiae (Baker's yeast), and Nedd8 modification has now emerged as a regulatory pathway of fundamental importance for cell cycle control and for embryogenesis in metazoans. The only identified Nedd8 substrates are cullins. Neddylation results in covalent conjugation of a Nedd8 moiety onto a conserved cullin lysine residue.

The inventors have now discovered, inter alia, that SMN stability is regulated by a GSK3/Cul1 phosphorylation-ubiquitination pathway. While GSK3 inhibition had little effect on mRNA levels or splicing of SMN, the inventors discovered that a GSK3 phosphorylation site on Ser4 of SMN regulates SMN stability. Without wishing to be bound by a theory, a cullin, e.g., cul1, interacts with phosphorylated SMN protein leading to degradation of the SMN protein by proteasome. Blocking the dephosphorylation of the SMN leads to reduction in degradation of the SMN protein via a reduction in cullin-mediated degradation of SMN protein.

Accordingly, certain aspects described herein relate to methods for regulating survival of motor neuron (SMN) protein levels. In an aspect, a method of regulating SMN protein levels in a cell comprises contacting the cell with an effective amount of an agent which inhibits ubiquitination of SMN protein in the cell, thereby regulating SMN protein levels in the cell.

In an aspect, a method of regulating SMN protein levels in a cell comprises contacting the cell with an effective amount of an agent which modulates ubiquitin ligase activity of a cullin in the cell, thereby regulating SMN protein levels in the cell.

In an aspect, a method of regulating SMN protein levels in a cell comprises contacting the cell with an effective amount of an spot which inhibits the level or activity of Nedd8 activating enzyme (NAE) in the cell, thereby regulating SMN protein levels in the cell.

As used herein, “regulating SMN protein levels” refers to modulation of SMN protein levels in any way which would be desirable to impart a beneficial effect to a cell comprising SMN protein. In some embodiments, regulating SMN protein levels in the cell comprises increasing the levels of SMN protein in the cell. In some embodiments, regulating SMN protein levels in the cell comprises increasing for levels of SMN protein in the cell without increasing the levels of SMN mRNA in the cell. In some embodiments, regulating SMN protein levels in the cell comprises stabilizing levels of SMN protein in the cell. In some embodiments, regulating SMN protein levels in the cell comprises reducing degradation of SMN protein in the cell. In some embodiments, regulating SMN protein levels in the cell comprises preventing degradation of SMN protein in the cell. In some embodiments, regulating SMN protein levels in the cell comprises genetically modifying the cell to express high levels of SMN protein. In some embodiments, regulating SMN protein levels comprises modulating alternative splicing of SMN mRNA to shift the balance of SMN2 splicing toward the production of full-length SMN2 messenger RNA.

In an aspect, the disclosure provides a method of regulating degradation of SMN protein in a cell (e.g., a neuron, e.g., motor neuron), in an embodiment, a method of regulating degradation of SMN protein in a cell comprises contacting a cell with an agent which inhibits ubiquitination of SMN protein in the cell, thereby regulating degradation of SMN protein in the cell.

In an embodiment, a method of regulating degradation of SMN protein in a cell comprises contacting a cell with an agent which modulates ubiquitin ligase activity of a cullin in the cell, thereby regulating degradation, of SMN protein in the cell.

In an embodiment, a method of regulating degradation of SMN protein in a cell comprises contacting a cell with an agent that inhibits the level or activity of Nedd8 activating enzyme (NAE) in the cell, thereby regulating degradation of SMN protein in the cell.

In an aspect, provided herein is a method of increasing SMN level in a cell, comprising contacting cell with an agent that inhibits ubiquitination of SMN protein in the cell.

In an aspect, provided herein is a method of increasing SMN level in a cell, comprising contacting cell with an agent that modulates the activity of a cullin, e.g., Cul1.

In an aspect, provided herein is a method of increasing SMN level in a cell, comprising contacting cell with an agent that inhibits the level or activity of Nedd8 activating enzyme (NAE) in the cell.

Certain aspects herein relate to methods of promoting cell survival. In an aspect, a method of promoting cell survival comprises contacting the cell with an effective amount of an agent which inhibits ubiquitination of SMN protein in the cell, thereby promoting survival of the cell.

In an aspect, a method of promoting cell survival comprises contacting the cell with an effective amount of an agent which modulates ubiquitin ligase activity of a cullin in the cell, thereby promoting survival of the cell.

In an aspect, a method of promoting cell survival comprises contacting the cell with an effective amount of an agent which inhibits the level or activity of Nedd8 activating enzyme (NAE) in the cell, thereby promoting survival of the cell.

Certain aspects provided herein relate to methods of promoting motor neuron survival. In an embodiment, a method of promoting motor neuron survival comprises contacting a motor neuron with an agent which inhibits ubiquitination of SMN protein in the motor neuron, thereby promoting survival of the motor neuron. In an embodiment, a method of promoting motor neuron survival comprises contacting a motor neuron with an agent which modulates the activity of a cullin in the motor neuron, thereby promoting survival of the motor neuron. In an embodiment, a method of promoting motor neuron survival comprises contacting a motor neuron with an agent which inhibits the level or activity of NAE in the motor neuron, thereby promoting survival of the motor neuron.

As used herein, the phrase “promoting cell survival” refers to an increase in survival of cells (e.g., neurons, e.g., motor neurons) as compared to a control. In some embodiments, contacting of a cell, (e.g., neurons, e.g., motor neurons) with an agent or compound described herein results in at least about 10%, 20%, 30%, 40%, 50%, 60%, 70%, 80%, 90%, 95%, 100%, 2-fold, 3-fold, 4-fold, 5-fold or more increase in cell survival (e.g., neuron, e.g., motor neuron) relative to non treated control.

Motor neuron survival can be assessed by for example (i) increased survival time of motor neurons in culture; (ii) increased production of a neuron-associated molecule in culture or in vivo, e.g., choline acetyltransferase, acetylcholinesterase. SMN or GEMs; or (iii) decreased symptoms of motor neuron dysfunction in vivo. Such effects may be measured by any method known in the art. In one non-limiting example, increased survival of motor neurons may be measured by the method set forth in Arakawa et al. (1990, J. Neurosci. 10:3507-3515); increased production of neuron-associated molecules may be measured by bioassay, enzymatic assay, antibody binding. Northern blot assay, etc., depending on the molecule to be measured; and motor neuron dysfunction may be measured by assessing the physical manifestation of motor neuron disorder. In one embodiment, the increase in motor neuron survival can be assessed by measuring the increase in SMN protein levels. Cell survival can also be measured by uptake of calcein AM, an analog of the viable dye, fluorescein diacetate. Calcein is taken up by viable cells and cleaved intracellularly to fluorescent salts which are retained by intact membranes of viable cells. Microscopic counts of viable neurons correlate directly with relative fluorescence values obtained with the fluorimetric viability assay. This method thus provides a reliable and quantitative measurement of cell survival in the total cell population of a given culture (Bozyczko-Coyne et al., J. Neur. Meth. 50:205-216, 1993). Other methods of assessing cell survival are described in U.S. Pat. Nos.: 5,972,639; 6,077,684 and 6,417,160, contents of which are incorporated herein by reference.

In vivo motor neuron survival can be assessed by an increase in motor neuron, neuromotor or neuromuscular function in a subject, in one non-limiting example, motor neuron survival in a subject can be assessed by reversion, alleviation, amelioration, inhibition, slowing down or stopping of the progression, aggravation or severity of a condition associated with motor neuron dysfunction or death in a subject, e.g., SMA or ALS.

Certain aspects disclosed herein relate to promoting cell survival in a subject in need thereof by regulating the overall levels of SMN protein in the subject. In an aspect, a method of promoting cell, survival in a subject in need thereof by regulating the overall levels of SMN protein in the subject comprises administering to a subject an effective amount of an agent which inhibits ubiquitination of SMN protein in the subject's cells, thereby promoting cell survival in the subject.

In an aspect, a method of promoting cell survival in a subject in need thereof by regulating the overall levels of SMN protein in the subject comprises administering to a subject an agent which modulates ubiquitin ligase activity of a cullin in the subject's cells, thereby promoting cell survival in the subject.

In an aspect, a method of promoting cell survival in a subject in need thereof by regulating the overall levels of SMN protein in the subject comprises administering to a subject an agent, which inhibits the level or activity of Nedd8 activating enzyme (NAE) in the subject's cells, thereby promoting cell survival in the subject.

Those skilled in the art will appreciate that regulating the overall levels of SMN protein in a subject can be achieved in a variety of ways. The disclosure contemplates any method which is capable of regulating the overall levels of SMN protein in a subject. In some embodiments of this and any aspect described herein, regulating the overall levels of SMN protein in the subject comprises increasing the levels of SMN protein in the subject's cells. In some embodiments of this and any aspect described herein, regulating the overall levels of SMN protein in the subject comprises stabilizing levels of SMN protein in the subject's cells. In some embodiments of this and any aspect described herein, regulating the overall levels of SMN protein in the subject comprises reducing degradation of SMN protein in the subject's cells. In some embodiments of this and any aspect described herein, regulating the overall levels of SMN protein in the subject comprises preventing degradation of SMN protein in the subject's cells. In some embodiments of this and any aspect described herein, regulating the overall levels of SMN protein in the subject comprises increasing the number of high SMN expressing cells in the subject. In some embodiments of this and any aspect described herein, regulating the overall levels of SMN protein in the subject comprises stabilizing the number of high SMN expressing cells in the subject. In some embodiments of this and any aspect described herein, regulating the overall levels of SMN protein in the subject comprises increasing the ratio of high SMN expressing cells to low SMN expressing cells in the subject. In some embodiments of thus and any aspect described herein, regulating the overall levels of SMN protein in the subject comprises decreasing the number of low SMN expressing cells in the subject.

### Agents and Compounds

The disclosure contemplates various agents and compounds which are useful in regulating the overall levels of SMN protein in a cell or subject for use in the methods, compositions, and kits described herein. It is contemplated that any agent which is useful for regulating the overall levels of SMN protein in a cell or subject can be used, for example, any agent which increases the levels of SMN protein, stabilizes levels of SMN protein, reduce degradation of SMN protein, prevents degradation of SMN protein, increases the number of high SMN expressing cells, stabilizes the number of high SMN expressing cells, increases the ratio of high SMN expressing cells to low SMN expressing cells, and/or decreases the number of low SMN expressing cells in the subject cast be used is the methods, compositions, and kits described herein.

Exemplary agents include small organic or inorganic molecules, saccharines, oligosaccharides, polysaccharides, biological macromolecules, peptides, proteins, peptide analogs and derivatives, peptidomimetics, antibodies fragments or portions of antibodies, nucleic acids, nucleic acid analogs and derivatives, an extract made from biological materials, animal tissues, naturally occurring or synthetic compositions, and any combinations thereof which are capable of achieving the desired effects in a cell or subject.

In some embodiments, the agent is a compound of formula (I):

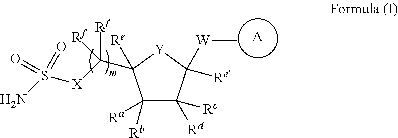

wherein;

Ring A is a 6-membered nitrogen-containing heteroaryl ring, optionally fused to a 5- or 6-membered aryl, heteroaryl, cycloaliphatic or heterocyclic ring, wherein either or both rings is optionally substituted and one ring nitrogen atom is optionally oxidized or Ring A is selected from the group consisting of

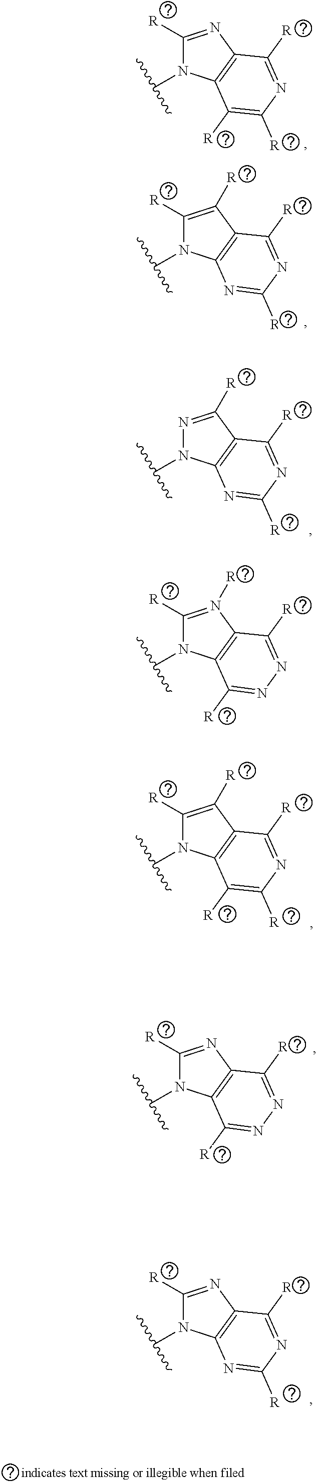

W is CH₂, CHF, CH₂, CH(R1), CH(R¹), NH, N(R), O, S, or —NHC(O)—;

R¹ is C₁₋₄ aliphatic or C₁₋₄ fluoroaliphatic; or R¹ is a C₂₋₄ alkylene chain that is attached to a ring position on Ring A to form a 5-, 6-, or 7-membered fused ring, wherein the alkylene chain optionally is substituted with C₁₋₄ aliphatic, C₁₋₄ fluoroaliphatic, —O, —CN, or —C(O)N(R⁴)2;

X is CH2, CHF, CH2, NH, or O;

Y is —O—, —S—, or —C(R^{m})(Rⁿ)—;

R^{a} is selected from the group consissting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;

R^{b} is selected from the group consisting of hydrogen, fluoro, C₁₋₄ aliphatic, and C₁₋₄ fluoroaliphatic;

R^{c} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;

R^{d} is selected from the group consisting of hydrogen, fluoro, C₁₋₄ aliphatic, and C₁₋₄ fluoroaliphatic;

R^{e} is hydrogen, or C₁₋₄ aliphatic; or R^{c}, taken together with one R^{f} and the intervening carbon atoms, forms a 3- to 6-membered spirocyclic ring; or R^{e}, taken together with R^{m} and the intervening carbon atoms, forms a fused cyclopropane ring, which is optionally substituted with one or two substituents independently selected from fluoro or C_{1-4 aliphatic;}

R^{e′} is hydrogen or C₁₋₄ aliphatic; or R^{e′}, taken together with R^{m} and the intervening carbon atoms, forms a fused cyclopropane ring, which is optionally substituted with one or two substituents independently selected from fluoro or C₁₋₄ aliphatic;

each R^{f} is independently hydrogen, fluoro, C₁₋₄ aliphatic, or C₁₋₄ fluoroaliphatic; or two R^{f} taken together form ═O; or two R^{f}, taken together with the carbon atom to which they are attached, form a 3- to 6-membered carbocyclic ring; or one R^{f}, taken together with R^{e} and the intervening carbon atoms, forms a 3- to 6-membered spirocyclic ring;

R^{m} is hydrogen, fluoro, —N(R⁴)₂, or an optionally substituted C₁₋₄ aliphatic group; or R^{m} and Rⁿ together form ═O or ═C(R⁵)₂; or R^{m} and R^{e}, taken together with the intervening carbon atoms, form a fused cyclopropane ring, which is optionally substituted with one or two substituents independently selected from fluoro or C₁₋₄ aliphatic; or R^{m} and R^{e′}, taken together with the intervening carbon atoms, form a fused cyclopropane ring, which is optionally substituted with one or two substituents independently selected from fluoro or C₁₋₄ aliphatic;

each Rⁿ is hydrogen, fluoro, or an optionally substituted C₁₋₄ aliphatic group; or R^{m} and Rⁿ together form ═O or ═C(R⁵)₂;

each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, to addition to the nitrogen atom, 0-2 heteroatoms independently selected from N, O, and S;

R^{4x} is hydrogen, C₁₋₄ alkyl, C₁₋₄ fluoroalkyl, or C₆₋₁₀ ar(C₁₋₄ )alkyl, the aryl portion of which can be optionally substituted;

R^{4y} is hydrogen, C₁₋₄ alkyl, C₁₋₄ fluoroalkyl, C₆₋₁₀ ar(C₁₋₄ )alkyl, the aryl portion of which can be optionally substituted, or an optionally substituted 5- or 6-membered aryl, heteroaryl, or heterocyclyl ring; or

R^{4x} and R4^{y}, taken together with the nitrogen atom to which they are attached, form an optionally substituted 4- to 8-membered heterocyclyl ring having, in addition to the nitrogen atom, 0-2 ring heteroatoms independently selected from N, O, and S; and

each R⁵ independently is hydrogen or an optionally substituted aliphatic, aryl, heteroaryl, or heterocyclyl group;

each R^{5x} independently is hydrogen, C₁₋₄ alkyol, C₁₋₄ fluoroalkyl, or an optionally substituted C₆₋₁₀ aryl or C₆₋₁₀ ar(C₁₋₄ )alkyl;

each R⁶ independently is an optionally substituted aliphatic, aryl, or heteroaryl group; R⁶ is hydrogen, halo, —NO₂, —CN, —C(R⁵)═C(R⁵)₂, —C═C—R, —OR⁵, —SR⁶, —S(O)R⁶—SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OCO₂R⁶, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁶, —N(R⁴)—N(R⁴)₂, —N(R⁴)—OR⁵, —C(═NR⁴)—N(R⁴)—OR⁵, —C(R⁶)═N—OR⁵, or an optionally substituted aliphatic, aryl, heteroaryl, or heterocyclyl;

each R^{h} independently is hydrogen, halo, —CN—, —OR⁵, —N(R⁴)², —SR⁶, or an optionally substituted C1-4 aliphatic group;

each R^{j} is hydrogen, —OR⁵, —SR⁶, —N(R⁴)₂, or an optionally substituted aliphatic, aryl, or heteroaryl group;

R^{k} is hydrogen, halo, —OR⁵, —SR⁶, —N(R⁴)—, or an optionally substituted C₁₋₄ aliphatic group;

m is 1, 2, or 3;

and pharmaceutically acceptable salts thereof.

Additional compounds of formula (I) are described in U.S. Patent Application Publication Nos. 2007/0191293, 2009/0036678, and U.S. Pat. No. 8,008,307, content of all of which is incorporated by reference in its entirety.

In some embodiments, the agent comprises the compound ((1S,2S,4R)-4-(4-(((S)-2,3-dihydro-1H-inden-1-yl)amino)-7H-pyrrolo[2,3-d]pyrimidin-7-yl)-2-hydroxycycloopentyl)methyl sulfamate (MLN4924). As used herein, “MLN”, “the MLN compound” and “MLN4924” are used interchangeably.

In some embodiments, the agent is an analog or derivative of MLN4924.

In some embodiments, the agent that modulates the activity of a cullin is a cullin polypeptide or a functional fragment thereof. The term “functional” when used in conjunction with “fragment” refers to a polypeptide which possesses a biological activity that is substantially similar to a biological activity of the entity or molecule of which it is a fragment thereof. By “substantially similar” in this context is meant that at least 25%, at least 35%, at least 50% of the relevant or desired biological activity of a corresponding wild-type peptide is retained. For example, a functional fragment of polypeptide retains enzymatic activity that is substantially similar to the enzymatic activity of the full length polypeptide. In some embodiments, the functional fragment of cullin polypeptide retains E3 ubiquitin ligase activity. Cullin polypeptide can comprise the amino acid sequence of human cullin-1, which can be accessed by accession no. Q13616 the UniprotKB database at www.uniprot.org/uniprot/.

In some embodiments, the agent that modulates the activity of a cullin is a cullin polypeptide (e.g., a dominant negative form of cullin in which a portion of the ammo acid sequence of the cullin polypeptide is truncated). An exemplary dominant negative form of a cullin polypeptide is the dominant negative Cul1-75 polypeptide described by Voigt and Papalopulu in which the last 75 amino acids of the cullin polypeptide are missing, including the neddylation site (Development, 2006; 133:559-568). It is believed that any dominant negative form of a cullin polypeptide which lacks the neddylation site can be used to modulate the activity of a cullin and stabilize SMN protein levels in a cell. In an embodiment, the cullin polypeptide comprises a mutant cullin polypeptide in which lysine 720 is missing. In some embodiments, the cullin polypeptide comprises a cullin polypeptide in which a sequence of many as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or up to 75 or more amino acids comprising lysine 720 are deleted from the cullin polypeptide. In some embodiments, a cullin polypeptide comprises a mutant polypeptide in which lysine 720 is mutated to a non-conservative amino acid residue. In some embodiments, a cullin polypeptide comprises a mutant polypeptide in which lysine 720 is mutated to a non-basic amino acid residue. In some embodiments, the cullin polypeptide comprises a mutant cullin polypeptide in which a neddylation site of the cullin polypeptide is mutated. In some embodiments, the neddylation site of the cullin polypeptide is mutated with a non-conservative amino acid substitution. In some embodiments, the neddylation site of the cullin polypeptide is mutated with a non-basic amino acid substitution.

In some embodiments, the agent can promote cell survival (e.g., neuron, e.g., motor neuron) by increasing SMN protein levels in cells. In some embodiments, the SMN protein levels are increased by about at least 5%, 10%, 20%, 30%, 40%, 50%, 60%, 70%, 80%, 90%, 95%, 100%, 1.1-fold, 1.25-fold, 1.5-fold, 1.75-fold, 2-fold, 3-fold, 4-fold, 5-fold, 10-fold or more relative to when cell is not contacted with a compound described herein.

In some embodiments, the agent can promote cell survival (e.g., neuron, e.g., motor neuron) survival by increasing SMN protein levels in cells. In some embodiments, the SMN protein levels are increased by about at least 5%, 10%, 20%, 30%, 40%, 50%, 60%, 70%, 80%, 90%, 95%, 100%, 1.1-fold, 1.25-fold, 1.5-fold, 1.75-fold, 2-fold, 3-fold, 4-fold, 5-fold, 10-fold or more relative to when cell is not contacted with a agent described herein.

In some embodiments, an increase in SMN protein levels can be accomplished without an increase in SMN RNA levels. For example, the agent or compound can act by inhibiting, preventing, blocking, stopping, or reducing degradation of SMN protein or by stabilizing the SMN protein against degradation. The compound can inhibit, prevent, block, stop, or reduce degradation of SMN protein by about at least 5%, 10%, 20%, 30%, 40%, 50%, 60%, 70%, 80%, 90%, 95%, 100%, 1.1-fold, 1.25-fold, 1.5-fold, 1.75-fold, 2-fold, 3-fold, 4-fold, 5-fold, 10-fold or more relative to when cell is not contacted with a the compound.

In some embodiments, the agent or compound sets by stabilizing the SMN protein against degradation. Without wishing to be bound by theory, the agent, or compound functions by decreasing or reducing activity of a cullin. In some embodiments, a compound described herein can decrease or reduce the activity of a cullin by at least 5%, 10%, 20%, 30%, 40%, 50%, 60%, 70%, 80%, 90%, 95%, 100%, 2-fold, 3-fold, 4-fold, 5-fold, 10-fold or more relative to in the absence of the compound.

In some embodiments, a cell (e.g., neuron, e.g., motor neuron cell) is contacted with a second compound, wherein the second compound modulates a biological pathway or target other than a cullin.

In some embodiments, the biological pathway can be selected from group consisting of PI-3K signaling pathway, Akt signaling pathway, MARK signaling pathway, PDGP pathway, RAS pathway, elF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof. In some embodiments, the signaling pathway is the PI-3/AKT/GSR pathway. In some embodiments, the pathway comprises GSK-3b, CDK2, CDK5, PKR or IKK-2b.

In some embodiments, the target is selected from the group consisting of Na⁺/K⁺ channel, MAPK, cannabinoid receptor, GPCR, Ca²⁺ channel, K⁺ channel, PDE5, GSK/CDK, PKR, CDK2, IKK-2, proteasome, BMP/TGFbeta receptor, dopamine receptor, and any combinations thereof.

In some embodiments, the second compound can be selected from the group consisting of RTK activator, insulin, FGF (e.g. FGF2), EGF, NGF, TGF (e.g. TGFβ), MAPK activator, kinase inhibitor, GSK inhibitor, CDK inhibitor, PKR inhibitor, IKK inhibitor, BMP/TGFβ ligand, cannabinoid or GPCR agonists, ion channel modulator (e.g. Na⁺/K⁺ channel modulator, Ca⁺ channel modulator, K⁺ channel modulator). PDE5 inhibitor, HDAC inhibitor, proteasome inhibitor, dopamine receptor ligand, PDGF, and combinations thereof.

Without wishing to be bound by theory, the second compound functions by increasing, inhibiting, preventing, blocking, stopping and/or reducing signaling activity in a biological pathway described herein. In some embodiments, the second compound described herein can alter the signaling activity by at least 5%; 10%, 20%, 30%, 40%, 50%, 60%, 70%, 80%, 90%, 95%, 100%, 2-fold, 3-fold, 4-fold, 5-fold, 10-fold or more relative to when pathway is not being modulated by the compound.

In some embodiments, the second compound is a GSK inhibitor, GSK inhibitors are known widely in the art and can be grouped into different chemical classes such as pyrroloazepine, flavone, beruazepinone, bis-indole, pyrrolopyrazine, pyridyloxadiazole, pyrazolopyridine, pyrazolopyridazine, aminopyridine, pyrazoloquinoxaline, oxindole (indolinone), thiazole, bisindolylmaleimide, azainodolylmaleimide, arylindolemaleimide, aniliomaleimide, phenylaminopyridine, triazole, pyrrolopyrimidine, pyrazolopyrimidine, and chloromethyl thienyl ketone. In some embodiments, the compound is a GSK inhibitor selected from the group consisting of CHIR98014, CHIR99021, GSK1, GSK2, GSK6, GSK7, GSK8 (ARA014418), GSK9, GSK10, GSK11, GSK12, GSK 13, GSK15, GSK 17, hymenialdisine, flavopiridol, aloisine A, aloisine B, pyrazolopyridine 18, pyrazolopyridine 9, pyrazolopyridine 34, CT20026, SU9516, staurosporine, GF109203x, RO318220, SB216763, SB415286, 15, CGP60474, and combinations thereof. Additional GSK-3beta inhibitors amenable to the compositions and methods described herein are described in U.S. Pat. Nos. 7,056,939, 7,045,519, 7,037,918, 6,989,382, 6,949,547, 6,872,737, 6,800,632, 6,780,625, 6,608,063, 6,489,344, 6,470,490, 6,441,053, 6,417,185, 6,323,029, 6,316,259, and 6,057,117, the contents of which each are incorporated herein by reference in their entirety.

In some embodiments, the second compound is a CDK inhibitor. Exemplary CDK inhibitors include, but are not limited to, 2-(3-Hydroxypropylamino)-6(O-hyddroxybenzylamino)-9-isopropylpurine, 2-bromo-12, 13-dihydro-5H-indolo[2,3-a]pyrrolo[3,4-c]carbazole-5,7(6H)-dione, 3-(2-Chloro-3-indolylmethylene)-1,3-dihydroindol-2-one, 2(bis-(Hydroxyethyl)amino)-6-(4methoxybenzylamino)-9-isopropyl-purine, 3-Amino-1Hpyrazolo[3,4-b]quinoxaline, 5-amino-3-((4-(aminosulfonyl)phenyl)amino)-N-(2,6-difluorophenyl)-1H1,2,4-triazole-1-carbothioamide, aloisine A, aloisine RP106, alsterpaullone 2-cyanoethyl, alvocidib, aminopurvalanol A, bohemine, CGP74514A, ethyl-(6-hydroxy-4-phenylbenzo [4,5]furo[2,3-b]pyridine-3-carboxylate, fisetin, N4-(6-Aminopyrimidin-4-yl)-sulfanilamide, flavopiridol, kenpaullone, NSC 625987, NU6102, NU6140, olomoucine, olomoucine II, roscovitine, SU9516, WR 216174 p21CiP1 (CDKN1A), p27KiP1, (CDKN1B), and analogs thereof. Additional CDK inhibitors are described in U.S. Pat. Nos. 7,084,271, 7,078,591, 7,078,525, 7,074,924, 7,067,661, 6,992,080, 6,939,872, 6,919,341, 6,710,227, 6,683,095, 6,677,345, 6,610,684, 6,593,356, 6,569,878, 6,559,152, 6,531,477, 6,500,846, 6,448,264, and 6,107,305, the contents of which each are incorporated herein by reference in their entirety.

In some embodiments, the second compound is a HDAC inhibitor. Inhibitors of HDAC include small molecular weight carboxylates (e.g., less than about 250 amu), hydroxamic acids, benzamides, epoxyketones, cyclic peptides, and hybrid molecules. (See, for example, Drummond D. C., et al. Annu. Rev. Pharmacol. Toxicol. (2005) 45Z:495-528, (including specific examples therein) which is hereby incorporated by reference in its entirety). Non-limiting examples HDAC inhibitors include, but are not limited to, Suberoylanilide Hydroxamic Acid (SAHA (e.g., MK0683, volinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (i.e., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (i.e., 6-(3-chlorophenylureido)caprole hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. HDAC inhibitors are commercially available, e.g., from BIOMOL International, Fukasawa, Merck Biosciences, Novartis, Gloucester Pharmaceuticals, Aton Pharma. Titan Pharmaceuticals, Schering AG, Pharmion, MethylGene, and Sigma Aldrich. Further HDAC inhibitors amenable to the invention include, but are not limited to, those that are described in U.S. Pat. Nos.: 7,183,298; 6,512,125; 6,541,661; 6,531,472; 6,960,685; 6,897,220; 6,905,669; 6,888,207; 6,800,638 and 7,169,801, and U.S. patent application Ser. Nos. 10/811,332; 12/286,769; 11,365,268; 11/581,570; 10/509,732; 10/546,153; 10/381,791 and 11/516,620, the contents of which each are incorporated herein by reference in their entirety.

In some embodiments, the second compound is a proteasome inhibitor. Exemplary proteasome inhibitors amenable to the invention include, but are not limited to those that are described in U.S. Pat. Nos. 5,693,617; 5,780,454; 5,834,487; 6,465,433; 6,794,516; 6,747,150; 6,117,887; 6,133,308; 6,6617,317; 6,294,560; 6,849,743; 6,310,057; 6,566,553; 6,075,150; 6,083,903; 6,066,730; 6,297,217 and 6,462,019, the contents of which each are incorporated herein by reference in their entirety. In one embodiment, the proteasome inhibitor is not lacacystin or those described in U.S. Pat. Publication No. 2007/0207144.

In some embodiments, the second compound is a dopamine receptor ligand. Exemplary dopamine receptor ligands amenable to the invention include, but are not limited to those that are described in U.S. Pat. Nos. 6,469,141; 5,998,414; 6,107,313; 5,849,765; 5,861,407; 5,798,350; 6,103,715; 5,576,314; 5,538,965; 5,968,478; 5,700,445; 5,407,823 and 5,602,121, the contents of which each are incorporated herein by reference in their entirety.

In some embodiments, the second compound is a cannabinoid (CB) receptor agonist. In one embodiment, the cannabinoid receptor agonist is WIN55,212-2 or anandamide. Other exemplary cannabinoid receptor agonists amenable to the invention include, but are nor limited to those that are described in U.S. Pat. Nos. 5,607,933; 5,324,737; 5,013,837; 5,081,122; 6,825,209; 5,817,651; 7,057,076; 5,068,234; 5,605,906; 6,995,187; 6,166,066;; 6,509,367; 6,100,259; 7,119,108; 4,973,587; 5,112,820; 7,037,910; 5,948,777; 5,925,768; 6,013,648; 6,864,291; 6,903,137; 6,943,266 and 5,596,106, the contents of which each are incorporated herein by reference in their entirety.

In some embodiments, the second compound is FGF, EGF, NGF, TGF, PDGF, PDGF-BB or insulin, which compound activates PI-3K signaling pathway.

In some embodiments, the second compound is an activator of PI-3K pathway, which compound activates PI3K, PDK or PKB.

In some embodiments, the second compound inhibits IκB kinase 2 (IKK-2). Exemplary IKK-2 inhibitors include, but are not limited to SC-514, SPC-839, IKK-2 inhibitor IV (CAS: 507475-17-4) and IKK-2 inhibitor VI. Other IKK-2 inhibitors amenable to the present invention include those described in U.S. pat. Nos. 7,122,544; 6,462,036; and 7,125,896, and U.S. patent application Ser. Nos. 11/271,598; 11/211,383; 10/542,044; 11/272,401; 11/430,215; 11/346,986; and 10/542,326, the contents of which each are incorporated herein by reference in their entirely. Further IKK-2 inhibitors are described in Bingham, A. H., et al., Bioorg. Med. Chem. (2003), 14, 409-412 and Liddle, J., et al., Bioorg. Med. Chem. Lett. (2009), 19, 2504-2508, the contents of which each are incorporated herein by reference in their entirety.

In some embodiments, the second compound is a modulator of TGF-β signaling. Exemplary modulators of TGF-62 signaling include, but are not limited to, AP-12009 (TGF-β Receptor type II antisense oligonucleotide), Lerdelimumab (CAT 152, antibody a TGF-β Receptor type II) GC-1008 (antibody to all isoforms of human TGF-β), ID11 (antibody to all isoforms of murine TCF-β, soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g., 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzol[1,3]dioxol-5-yl-4-pyridin-2-yl-1H-imidazol-2-yl)-benzamide) GW788388 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, lefty 1, Lefty 2, Follistatin, Noggin, Chordin, Cerberus, Gremlin, inhibin, BIO (6-bromo-indirubin-3′-oxime), Smad proteins (e.g., Smad6, Smad7), Cystatin C, soluble TGF-β Receptor type I, AP-11014 (TOF-β Receptor Type I antisense oligonucleotide), Metelimumab (CAT 152, TGF-β Receptor type I antibody), LY550410, LY580276 (3-(4-fluorophenyl)-5,6-dihydro-2-(6-methylppyridin-2-yl)-4H-pyrrolo[1,2-b]pyrazole, LY364947 (4-[3-(2-Pyridinyl)-1H-pyrazol-4-yl]-quinoline), LY2109761, LY573636 (N-((5-bromo-2-thienyl)sulfonyl)-2,4-dichlorobenzamide), SB-505124 (2-(5-Benzo[1,3]dioxol-5-yl-2-tert-butyl-3H-imidazol-4-yl)-6-methylpyridine), SD-208 (2-(5-Chloro-2-fluorophenyl)-4-[(4-pyridyl)amino]pteridine), SD-093, KI2689, SM16, FKBP12 protein, 3-(4-(2-(6-methypyridin-2-yl)H-imidazo[1,2-a]pyridin-3-yl)quinolin-7-yloxy)-N,N-dimethylpropan-1-amine, and analogs thereof. Other modulators of TGF-β signaling amenable to the invention are described in Callahan, J. F. et al., J. Med. Chem. 45, 999-1001 (2002); Sawyer, J. S. et al., J. Med. Chem. 46,3953-3956 92003); Sawyer, J. S. et al., Bioorg. Med. Chem. Lett. 14, 3581-3584 (2004); Gellibert, F. et al., J. Med. Chem. 47, 4494-4506 (2004); Yingling, J. M. et al., Nature Rev. Drug Disc. 3, 1011-1022 (2004); Tojo, M. et al., Cancer Sci. 96: 791-800 (2005); Valdimarsdottir, G. et al., APMIS 113, 773-389 (2005); Petersen et al. Kidney International 73, 705-715 (2008); yingling, J. M. et al., Nature Rev. Drug Disc. 3, 1011-1022 92004); Byfield, S. D. et al., Mol. Pharmacol., 65, 744-752 (2004); Byfield, S. D., and Roberts, A. B., Trends Cell Biol. 14, 107-111 (2004); Dumont, N. et al., Cancer Cell 3, 531-536 (2003); WO Publication No. 2002/094833; WO Publication No. 2004/026865; WO Publication No. 2004/067530; WO Publication No. 209/032667; WO Publication No. 2004/013135; WO Publication No. 2003/097639; WO Publication No. 2007/048857; WO Publication No. 2007/018818; WO Publication No. 2006/018967; WO Publication No. 2005/039570; WO Publication No. 2000/031135; WO Publication No. 1999/058128; WO Publication Mo. 2004/926871; WO Publication No. 2004/021989; WO Publication No. 2004/026307; WO Publication No. 2000/012497; U.S. Pat. No. 6,509,318; U.S. Pat. No. 6,090,383; U.S. Pat. No. 6,419,928; U.S. Pat. No. 9,927,738; U.S. Pat. No. 7,223,766; U.S. Pat. No. 6,476,031: U.S. Pat. No. 6,419,928: U.S. Pat. No. 7,030,125; U.S. Pat. No. 6,943,191; U.S. Pat. No. 5,731,144; U.S. Pat. No. 7,151,169; U.S. Publication No. 2005/0245520; U.S. Publication No. 2004/0147574; U.S. Publication No. 2007/0066632; U.S. Publication No. 2003/0028905; U.S. Publication No. 2005/0032835; U.S. Publication No. 2008/0108656; U.S. Publication No. 2004/015781; U.S. Publication No. 2004/0204431; U.S. Publication No. 2006/0003929; U.S. Publication No. 2007/0155722; U.S. Publication No. 2004/0038856; U.S. Publication No. 2005/0245508; U.S. Publication No. 2004/0138188 and U.S. Publication No. 2009/0036382, contents of all of which are herein incorporated in their entireties. Oligonucleotide based modulators of TGF-β signaling, such as siRNAs and antisense oligonucleotides, are described in U.S. Pat. No. 5,731,424; U.S. Pat. No. 6,124,449; U.S. Publication Nos. 2008/0015161; 2006/0229266; 2004/0006030; 2005/0227936 and 2005/0287128, contents of all of which are herein incorporated in their entireties. Other antisense nucleic acids and siRNAs can be obtained by methods known to one of ordinary skill in the art.

In some embodiments, BMP/TGFβ ligand is BMP4 (bone morphogenetic protein 4). Other exemplary BMP/TGFβ modulators are described in U.S. Pat. Nos.: 7,223,766 and 7,354,722, the contents of which each are incorporated herein by reference in their entirety.

In some embodiments, second compound is a Na⁺, K⁺ and/or Ca²⁺, ion channel modulator. Exemplary ion channel modulators are described in U.S. pat. Nos.: 6,184,231; 6,479,498; 6,646,012; 5,565,483; 5,871,940; 6,172,085; 5,242,947; 7,132,422; 6,756,400; 7,183,323; 7,226,950 and 6,872,741, and U.S. patent application Ser. Nos.: 11/434,920; 11/546,669; 10/514,150; 11/450,695; 11/216,376; 10/743,280; 11/434,627; 10/977,609; 11/811,909; 11/418,163; 11/556,354; 11/432,997; 11/216,899; 11/517,754; 11/418,278; 10/792,688; 11/574,751; 11/643,622; 11/266,142; 10/935,008; 12/158,491; 10/450,215; 10/427,847 and 10/838,087, the contents of which each are incorporated herein by reference in their entirety. In some embodiments, Na⁺/K⁺ channel modulator is a cardiac glycoside selected from the group consisting of Ouabain, Digoxin, Dititoxin, Lanatoside C, and combinations thereof. In some embodiments, Ca²⁺ channel modulator is Thapsigargin, ionomycin or Calcimycin. In some embodiments, Ca²⁺ channel modulator is Veratridine, Monensin NA or Valinomycin.

In some embodiments, MAPK activator is Anysomycin or Coumermycin.

Exemplary inhibitors of PDE5 are described in U.S. Pat. Nos.; 6,869,974; 6,680,047; 6,635,274; 6,555,663 and 6,472,425, the contents of which each are incorporated herein by reference in their entirety. In some embodiments, PDE5 inhibitor is selected from the group consisting of MBCQ, Dipyridamole, spironolactone, bucladesine, and combinations thereof.

In some embodiments, the second compound is an activator of RTK signaling. Exemplary modulators of RTK signaling are described in U.S. Pat. Nos.: 5,196,446; 5,374,652; 6,316,635; 7,214,700; 6,569,868; 5,302,606; and 6,849,641, the contents of which each are incorporated herein by reference is their entirety. In some embodiments, RTK activator is PDGF-BB.

In some embodiments, the second compound is a growth factor.

In some embodiments, the second compound inhibits the activity of at least one kinase. In some embodiments, the kinase is selected from the group consisting of phosphoinositide 3-kinases (PI-3 kinases), phosphoinositide dependent kinase 1 (PDK1), SGK, glycogen synthase kinase 3 (GSK-3), inhibitor of IκB kinase 2 (IKK2), cyclin dependent kinase 2 (CDK2), and RNA dependent protein kinase.

In some embodiments, the second compound inhibits the activity of at least two different kinases. For example, the second compound can inhibit the activity of GSK-3 and a second kinase. In some embodiments, the second kinase is CDK.

Without limitations, cullin modulators and other agents or compounds described herein can be small organic or inorganic molecules (i.e., including heteroorganic and organometallic compounds), saccharines, oligosaccharides, polysaccharides, biological macromolecules, peptides, proteins, peptide analogs and derivatives, peptidomimetics, antibodies, fragments or portions of antibodies, nucleic acids, nucleic acid analogs and derivatives, an extract made from biological materials (such as bacteria, plants, fungi, or animal cells) animal tissues, naturally occurring or synthetic compositions, and any combinations thereof.

Nucleic acid based cullin modulators and nucleic acid based modulators of biological pathways and targets include, but are not limited to, antisense oligonucleotide, siRNA, shRNA, ribozyme, aptamers, decoy oligonucleotides. Methods of preparing such nucleic acids are known in the art and easily available to those skilled in the art.

Amino acid based cullin modulators and amino acid based modulators of biological pathways and targets include, but are not limited to, peptides, oligopeptides, proteins, peptidomimetics, antibodies, and portions or fragments of antibodies. As used herein, the term “antibody” includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)₂, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites. Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv beads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific. Thus, antibodies useful in the methods described herein include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with an antigen. While both polyclonal and monoclonal antibodies can be used in the methods described herein, it is preferred that a monoclonal antibody is used where conditions require increased specificity for a particular protein. Antibodies can be raised against a biological pathway component or target by methods known to those skilled in the art. Such methods are described in detail, for example, in Harlow et al., 1988 in: Antibodies, A Laboratory Manual, Cold Spring Harbor, N.Y.

Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

### Motor Neurons

Motor neurons for the aspects disclosed herein can be obtained from any source available to one of skill at the art. Additionally, a motor neuron can be of any origin. Accordingly, in some embodiments, the motor neuron is a mammalian motor neuron. In one embodiment, the motor neuron is a human motor neuron or a mouse motor neuron.

A rapid and efficient protocol for directed differentiation of mouse embryonic stem (ES) cells into functionally normal motor neurons has been well established by Wichterle's laboratory utilizing sequential treatment with retinoic acid and a Hh agonist (Wichterle et al. Cell (2002) 110:385; Wichterle and Peljto. Curr Protoc Stem Cell Biol (2008) vol. Chapter 1 pp. Unit 1H.1.1-1H.1.9). This has allowed the generation of sufficiently large numbers of motor neurons (Kris et al., Stem Cell Res, 2011, 6:195). Accordingly, in some embodiments, motor neuron is a human ES cell-derived motor neuron.

In some embodiments, the BS cell is from a subject, e.g., a patient. In some embodiments, the subject, e.g., a patient, is suffering from a neurodegenerative disorder. In some embodiments, the subject, e.g., a patient, is suffering from a neurodegenerative disorder characterized by degradation of SMN protein. In some embodiments, the subject, e.g., a patient, is suffering from a neurodegenerative disorder characterized by diminished levels of SMN protein. In some embodiments, the neurodegenerative disorder is ALS. In some embodiments, the neurodegenerative disorder is SMA. In one embodiment, the ES cell is from a carrier, e.g., a symptom-free carrier.

In some embodiments, the motor neurons comprise a mutation in a gene associated with a neurodegenerative disorder. One non-limiting example of a gene associated with a neurodegenerative disorder is SMN1. In another non-limiting example of a gene associated with a neurodegenerative disorder is SMN2. Still, another non-limiting example of a gene associated with a neurodegenerative disorder is SOD1. A variety of SOD1 mutant alleles are known to be associated with SMA and/or ALS, including without limitation, SOD1G93A.

In some embodiments, methods of the invention employs cells that are not neurons, e.g., non-neuronal cells. In some embodiments, methods of the invention employs cells that are not motor neurons, e.g., non-motor neuron cells. In some embodiments, the cells can comprise a mutation in a gene associated with a neurodegenerative disease. In some embodiments, methods of the present invention employ fibroblasts. In some embodiments, methods of the present invention employ HEK cells.

In some embodiments, the fibroblast comprises a mutation in a gene associated with a neurodegenerative disease. In some embodiments, methods of the invention employ non-motor neuron cells (e.g., fibroblasts or HBK cells) comprising a mutation in a SOD1 gene, such as, without limitation, SOD1G93A.

As used herein, the term “SOD1” refers to either the gene encoding superoxide dismutase 1 or the enzyme encoded by this gene. The SOD1 gene or gene product is known by other names in the art including, but not limited to, ALS1 Cu/Zn superoxide dismutase, indophenoloxidase A, IPOA, and SODC_HUMAN. Those of ordinary skill in the art will be aware of other synonymous names that refer to the SOD1 gene or gene product. The SOD1 enzyme neutralizes supercharged oxygen molecules (called superoxide radicals), which can damage cells if their levels are not controlled. The human SOD1 gene maps to cytogenetic location 21q22.1. Certain mutations in SOD1 are associated with ALS in humans including, but not limited to, Ala4Val, Gly37Arg and Gly93Ala, and more than one hundred others. Those of ordinary skill in the art will be aware of these and other human mutations associated with ALS. Certain compositions and methods of the present invention comprise or employ cells comprising a SOD1 mutation.

“SOD 1 mutations” refer to mutations in the SOD1 gene (NC_{—}000021.8; NT_{—}011512.11; AC_{—}000064.1; NW_{—}927384.1; AC_{—}000153.1; NW_{—}001838706.1 NM_{—}000454.4; NP_{—}000445.1 and NCBI Entrez GeneID: 6647) including but are not limited to Ala4Val, Cys6Gly, Val7Glu, Leu8Val, Gly10Val, Gly12Arg, Val14Met, Gly16Ala, Asn19Ser, Phe20Cys, Glu21Lys, Gln22Leu, Gly37Arg, Leu38Arg, Gly41Ser, His43Arg, Phe45Cys, His 46arg, Val47Phe, His48Gln, Glu49Lys, Thr54KArg, Ser59Ile, Asn65Ser, Leu67Arg, Gly72Ser, Asp76 Val, His80Arg, Leu84Phe, Gly85Arg, Asn86Asp, Val87Ala, Ala89Val, Asp90Ala, Gly93Ala, Ala 95Thr, Asp96Asn, Val97Met, Glu100Gly, Asp101Asn, Ile104Phe, Ser105Leu, Leu106Val, Gly108Val, Ile112Thr, Ile113Phe, Gly114Ala, Arg115Gly, Val118Leu, Ala140Gly, Ala145Gly, Asp124Val, Asp124Gly, Asp125His, Leu126Ser, Ser134Asn, Asn139His, Asn139Lys, Gly141Glu, Leu144Phe, leu144Ser, Cys146Arg, Ala145Thr, gly147Arg, Val148Gly, Val148Ile, Ile149thr, Ile151Thr, and Ile151Ser. SOD1 is also known as ALS, SOD, ALS1, IPOA, homodimer SOD1. “SOD 1 mutation” databases can be found at Dr. Andrew C. R. Martin website at the university College of London (www.bioinfo.org.uk), the ALS/SOD1 consortion website (www.alsod.org) and the human gene mutation database (HGMD®) at the Institute of Medical Genetics at Cardiff, united Kingdom.

In some embodiments, the cell is a SMN deficient cell. By “SMN deficient cell” is meant a cell that has at least partially reduced SMN levels of function compared to a healthy or normal reference level of SMN.

Contacting Cells with Agents

In practicing the subject methods, any cell that expresses SMN can be utilized. Non-limiting examples of specific cell types in which SMN protein levels can be modulated include fibroblast, cells of skeletal tissue (bone and cartilage), cells of epithelial tissues (e.g. liver, lung, breast, skin, bladder and kidney), muscle cells, skeletal muscle cells, cardiac and smooth muscle cells, neural cells (glia and neurons, e.g., motor neurons, cortical neurons, dopaminergic neurons, etc.), endocrine cells (adrenal, pituitary, pancreatic islet cells), melanocytes, and many different types of hematopoietic cells (e.g., cells of B-cell or T-cell lineage, and their corresponding stem cells, lymphoblasts). In some embodiments, the cell is a mammalian cells. In some embodiments, the cell is a human cell. In some embodiments, the cell is a HEK293T cell.

Cells (e.g., neurons, e.g., motor neurons) can be contacted with the agents described herein in a cell culture e.g., in vitro or ex vivo, or administrated to a subject, e.g., in vivo, in some embodiments of the invention, an agent described herein can be administrated to a subject to treat, prevent, and/or diagnose neurodegenerative disorders, including those described herein.

The term “contacting” or “contact” as used herein in connection with contacting a cell (e.g., neurons, e.g., motor neuron) includes subjecting the cell to an appropriate culture media which comprises the indicated compound or agent. Where the cell is in vivo, “contacting” or “contact” includes administering the compound or agent in a pharmaceutical composition to a subject via an appropriate administration route such that the compound or agent contacts the cell in vivo. Measurement of cell survival can be based on the number of viable cells alter period of time has elapsed after contacting of cells with a compound or agent, for example, number of viable cells can be counted after about at least 5 minutes, 10 minutes, 20 minutes, 30 minutes, 40 minute, 45 minutes, 50 minutes, 1 hour, 1.5 hours, 2 hours, 4 hours, 6 hours, 12 hours, 24 hours, 2 days, 3 days or more and compared to number of viable cells in a non-treated control.

For in vitro methods, cells (e.g., neurons, e.g., motor neurons) can be obtained from different sources, for example, neurons, e.g., motor neurons can be obtained boat a subject, or derived boot non neuronal cells from a subject. In some embodiments, motor neuron is a whole cell. In some embodiments, the cells are obtained from a subject. In some embodiments, the subject is suffering from a disorder characterized by degradation of SMN protein. In some embodiments, the subject is suffering from a disorder characterized by diminished levels of SMN protein. In some embodiments, the subject is suffering from a neurodegenerative disorder. In some embodiments, the subject is suffering from a neurodegenerative disorder characterized by degradation of SMN protein. In some embodiments, the subject is suffering from a neurodegenerative disorder characterized by diminished levels of SMN protein. In some embodiments, the subject is suffering from SMA. In some embodiments, the subject is suffering from ALS. In some embodiments, the subject is a carrier e.g., a symptom-free carrier. In some embodiments, cells are derived from a subject's embryonic stem cells (ESCs). In some embodiments, neurons are derived from a subject's ESCs. In some embodiments, motor neurons are derived from a subject's embryonic stem cells (ESCs). In some embodiments, the subject is a human. In some embodiments, the subject is a mouse. In some embodiments, the mouse is a transgenic mouse. Methods of inducing motor neuron differentiation from embryonic stem cells are known in the art, for example as described in Di Giorgio et al., Nature Neuroscience (2007), published online 15 Apr. 2007; doi:10.1038/nn1885 and Wichterle et al., Cell (2002) 110:385-397. In some instances induced pluripotent stem cells can be generated from a subject and then differentiated into cells (e.g., neurons, e.g., motor neurons). One exemplary method of deriving cells (e.g., motor neurons bono a subject is described in Dimos, J. T., et. al. Science (2008) 321, 1218-122 (Epub Jul. 31, 2008).

For in vivo methods, a therapeutically effective amount of a compound, composition, or agent described herein can be administered to a subject. Methods of administering compounds, compositions, and agents to a subject are known in the art and easily available to one of skill in the art.

### Method of Treatment

As one of skill in the art is aware, promoting survival of cells is a subject can lead to treatment, prevention, or amelioration of a number of disorders involving death of those cells. The inventors have discovered that cell death in certain cell types, such as neurons (e.g., motor neurons), is associated with degradation of, or diminished levels of SMN protein. In such instances, promoting survival of neurons (e.g., motor neurons) in a subject can lead to treatment, prevention or amelioration of a number of neurodegenerative disorders, particularly those neurodegenerative disorders in which cell death is associated with degradation of or diminished levels of SMN protein.

Accordingly, in an aspect, provided herein is a method of treating or preventing a disorder characterized by degradation of SMN protein in a subject in need thereof, comprising administering to the subject an effective amount of at least one agent which regulates degradation of SMN protein in a subject. In some embodiments, the at least one agent is an agent which inhibits ubiquitination of SMN protein. In some embodiments, the at least one agent is an agent which inhibits phosphorylation of SMN protein. In some embodiments, the at least one agent is an agent which inhibits neddylation of a cullin. In some embodiments, the at least one agent is an agent which inhibits neddylation of cullin1. In some embodiments, the at least one agent is an agent which modulates ubiquitin ligase activity of a cullin. In some embodiments, the at least one agent is an agent which inhibits the level or activity of NAE. In some embodiments, the disorder characterized by degradation of SMN protein is a neurodegenerative disorder. In some embodiments, the neurodegenerative disorder is SMA. In some embodiments, the neurodegenerative disorder is ALS.

In an aspect, provided herein is a method of treating or preventing a disorder characterized by diminished levels of SMN protein in a subject in need thereof, comprising administering to the subject an effective amount of at least one agent which increases the levels of SMN protein in a subject. In some embodiments, the at least one agent is an agent which inhibits ubiquitination of SMN protein. In some embodiments, the at least one agent is an agent which inhibits phosphorylation of SMN protein. In some embodiments the at least one agent is an agent which modulates ubiquitin ligase activity of a cullin. In some embodiments, the at least one agent is an agent which inhibits the level or activity of NAE. In some embodiments, the at least one agent is an agent which inhibits neddylation of a cullin. In some embodiments, the disorder characterized by degradation of SMN protein is a neurodegenerative disorder. In some embodiments, the neurodegenerative disorder is SMA. In some embodiments, the neurodegenerative disorder is ALS.

In an aspect, provided herein is a method of treating or preventing a neurodegenerative disorder in a subject in need thereof, comprising adminstering to the subject an effective amount of at least one agent which regulates degradation of SMN protein in a subject.

In an aspect, provided herein is a method of treating or preventing a neurodegenerative disorder in a subject in need thereof, comprising adminstering to the subject an effective amount of at least one agent which increases the levels of SMN protein in a subject.

In an aspect, provided herein is a method of treating or preventing a neurodegenerative disorder in a subject in need thereof, comprising administering to the subject at least one agent which regulates the overall levels of SMN protein in the subject. In some embodiments, regulating the overall levels of SMN protein in the subject comprises increasing the levels of SMN protein in the subject's cells. In some embodiments, regulating the overall levels of SMN protein in the subject comprises stabilizing levels of SMN protein in the subject's cells. In some embodiments, regulating the overall levels of SMN protein in the subject comprises reducing degradation of SMN protein in the subject's cells. In some embodiments, regulating the overall levels of SMN protein in the subject comprises preventing degradation of SMN protein in the subject's cells. In some embodiments, regulating the overall levels of SMN protein in the subject comprises increasing the number of high SMN expressing cells in the subject. In some embodiments, regulating the overall levels of SMN protein in the subject comprises stabilizing the number of high SMN expressing cells in the subject. In some embodiments, regulating the overall levels of SMN protein in the subject comprises increasing the ratio of high SMN expressing cells to low SMN expressing cells in the subject. In some embodiments, regulating the overall levels of SMN protein in the subject comprises decreasing the number of low SMN expressing cells in the subject.

In an aspect, provided herein is a method of treating or preventing a neurodegenerative disorder in a subject in need thereof, comprising administering to the subject at least one agent which increases the number of high SMN expressing cells in the subject. In some embodiments of this and any other aspect herein, high SMN expressing cells comprise high SMN expressing neurons. In some embodiments of this and any other aspect herein, high SMN expressing cells comprise high SMN expressing motor neurons.

In an aspect, provided herein is a method of treating or preventing a neurodegenerative disorder in a subject in need thereof, comprising administering to the subject at least one agent which decreases the number of low SMN expressing cells in the subject.

In aspects, provided herein is a method of treating or preventing a neurodegenerative disorder in a subject in need thereof comprising administering to the subject a therapeutically effective amount of at least one agent which increases the number of high SMN expressing cells in the subject and administering to the subject a therapeutically effective amount of at least one agent which decreases the number of low SMN expressing cells in the subject.

In some embodiments, a method of treating or preventing a neurodegenerative disorder in a subject in need thereof composes administering to the subject a therapeutically effective amount of a composition comprising a least one agent which increases the number of high SMN expressing cells in the subject and at least one agent which decreases the number of low SMN expressing cells in the subject.

In some embodiments of this and any other aspect herein, low SMN expressing cells comprise low SMN expressing neurons. In some embodiments of this and any other aspect herein, low SMN expressing cells comprise low SMN expressing motor neurons.

In some embodiments of this and any other aspect herein, the at least one agent which increases the number of high SMN expressing cells is an agent which inhibits ubiquitination of SMN protein. In some embodiments of this and any other aspect herein, the at least one agent which increases the number of high SMN expressing cells is an agent which inhibits phosphorylation of SMN protein. In some embodiments of this and any other aspect herein, the at least one agent which increases the number of high SMN expressing cells is an agent which modulates ubiquitin ligase activity of a cullin. In some embodiments of this and any other aspect herein, the at least one agent which increases the number of high SMN expressing cells is en agent which inhibits the level or activity of NAE. In some embodiments of this and any other aspect herein, the at least one agent which decreases the number of low SMN expressing cells is an HDAC inhibitor. In some embodiments of this and any other aspect herein, the at least one agent which decreases the number of low SMN expressing cells is an HDAC Type I inhibitor. In some embodiments of this and any other aspect herein, the at least one agent which decreases the number of low SMN expressing cells is an HDAC Type II inhibitor. In some embodiments of this and any other aspect herein, the at least one agent which decreases the number of low SMN expressing cells is an inhibitor of HDAC Type I and II. In some embodiments of this and any other aspect herein, the at least one agent which decreases the number of loot SMN expressing cells is an HDAC1 inhibitor. In some embodiments of this and any other aspect herein, the at least one agent which decreases the number of low SMN expressing cells is an HDAC2 inhibitor. In some embodiments of this and any other aspect herein, the at least one agent which decreases the number of low SMN expressing cells is an HDAC3 inhibitor. In some embodiments of this and any other aspect herein, the at least one agent which decreases the number of low SMN expressing cells is art HDAC8 inhibitor. In some embodiments of this and any other aspect herein, the at least one agent which decreases the number of low SMN expressing cells is an HDAC11 inhibitor. In some embodiments of this and any other aspect herein, the at least one agent which decreases the number of low SMN expressing cells is an inhibitor. In some embodiments of this and any other aspect herein, the at least one agent which decreases the number of low SMN expressing cells is an HDAC5 inhibitor. In some embodiments of this and any other aspect herein, the at least one agent which decreases the number of low SMN expressing cells is an HDAC6 inhibitor. In some embodiments of this and any other aspect herein, the at least one agent which decreases she number of low SMN expressing cells is an HDAC7 inhibitor. In some embodiments of this and any other aspect herein, the at least one agent which decreases the number of low SMN expressing cells is an HDAC9 inhibitor. In some embodiments of this and any other aspect herein, the at least one agent which decreases the number of low SMN expressing cells is an HDAC10 inhibitor. In some embodiments of this and any other aspect herein, the at least one agent which decreases the number of low SMN expressing cells is dual inhibitor of at least one HDAC Type I selected from the group consisting of HDAC1, HDAC2, HDAC3, HDAC8, and HDAC11, and at least one HDAC Type I selected from the group consisting of HDAC4, HDAC5, HDAC6, HDAC7, HDAC9 and HDAC10. In some embodiments of this, and any other aspect herein, the at least one agent which decreases the number of low SMN expressing cells is Trichostatin (e.g., Trichostatin A) or an analog or derivative thereof. Exemplary analogs of Trichostatin A and methods of synthesizing them are described in U.S. Patent Publication No. 2011/0237832, which is incorporated by reference herein. For example, the Trichostatin A analog comprises a compound having the following structure.

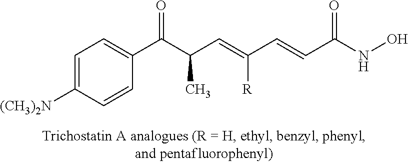

In some embodiments of this and any other aspect herein, the at least one agent which decreases the number of low SMN expressing cells is Apicidin ([cyclo-L-(2-Amino-8-oxodecanoyl)-L-(N-methoxy-tryptophan)-L-isoleucyl-D-pipecolinyl]) or an analog or derivative thereof. In some embodiments of this and any other aspect herein, the at least one agent which decreases the number of low SMN expressing cells is 5-Aza-2′-deoxycytidine or an analog or derivative thereof. In some embodiments of this and any other aspect herein, the at least one agent which decreases the number of low SMN expressing cells is CAY10433 or an analog or derivative thereof. In some embodiments of this and any other aspect herein, the at least one agent which decreases the number of low SMN expressing cells is CAY10398 or an analog or derivative thereof. In some embodiments of this and any other aspect herein, the at least one agent which decreases the number of low SMN expressing cells is 6-Chloro-2,3,4,9-tetrahydro--1H-carbazole-1-carboxamide or an analog or derivative thereof. In some embodiments of this and any other aspect herein, the at least one agent which decreases the number of low SMN expressing cells is HC Toxin or an analog or derivative thereof. In some embodiments of this and any other aspect herein, the at least one agent which decreases the number of low SMN expressing cells is ITSA1 or an analog or derivative thereof. In some embodiments of this and any other aspect herein, the at least one agent which decreases the number of low SMN expressing cells is M344 or an analog or derivative thereof. In some embodiments of this and any other aspect herein, the at least one agent which decreases the number of low SMN expressing cells is MC 1293 or an analog or derivative thereof. In some embodiments of this and any other aspect herein, the at least one agent which decreases the number of low SMN expressing cells is Oxamflatin or an analog or derivative thereof. In some embodiments of this and any other aspect herein, the at least one agent which decreases the number of low SMN expressing cells is PXD101 or an analog or derivative thereof. In some embodiments of this and any other aspect herein, the at least one agent which decreases the number of low SMN expressing cells is Scriptaid or an analog or derivative thereof. In some embodiments of this and any other aspect herein, the at least one agent which decreases the number of low SMN expressing cells is Sodium butyrate or an analog or derivative thereof. In some embodiments of this and any other aspect herein, the at least one agent which decreases the number of low SMN expressing cells is Sodium 4-phenylbutyrate or an analog or derivative thereof. In some embodiments of this and any other aspect herein, the at least one agent which decreases the number of low SMN expressing cells is Splitomicin or an analog or derivative thereof. In some embodiments of this and any other aspect herein, the at least one agent which decreases the number of low SMN expressing cells is Valproic acid or an analog or derivative thereof (e.g., Valproic acid sodium salt). In some embodiments, the disorder characterized by degradation of SMN protein is a neurodegenerative disorder. In some embodiments, the neurodegenerative disorder is SMA. In some embodiments, the neurodegenerative disorder is ALS.

In an aspect, provided herein is a method of treating or preventing a neurodegenerative disorder in a subject in need thereof, comprising administering to the subject at least one agent which inhibits ubiquitination of SMN protein.

In an aspect, provided herein is a method of treating or preventing a neurodegenerative disorder in a subject in need thereof, comprising administering to the subject at least one agent which modulates ubiquitin ligase activity of a cullin.

In an aspect, provided herein is a method of treating or preventing a neurodegenerative disorder in a subject in need thereof comprising administering to the subject at least one agent which inhibits the level or activity of Nedd8 activating entwine (NAE).

In an aspect, provided herein is a method of treating or preventing a neurodegenerative disorder in a subject in need thereof, comprising: (a) obtaining a biological sample comprising cell is from a subject suspected of having a neurodegenerative disorder; (b) contacting the cells with an effective amount of at least one agent which regulates the overall level of SMN protein in the cells, and (c) administering the cells to the subject, thereby treating or preventing the neurodegenerative disorder.

In an aspect, provided herein is a method of treating or preventing a neurodegenerative disorder in a subject in need thereof, comprising: (a) obtaining a biological sample comprising cells from a subject suspected of having a neurodegenerative disorder; (b) contacting the cells with an effective amount of at least one agent which inhibits ubiquitination of SMN protein in the cells, and (c) administering the cells to the subject, thereby treating or preventing the neurodegenerative disorder.

In an aspect, provided herein is a method of treating or preventing a neurodegenerative disorder in a subject in need thereon comprising: (a) obtaining a biological sample comprising cells from a subject suspected of having a neurodegenerative disorder; (b) contacting the cells with an effective amount of at least one agent which modulates ubiquitin ligase activity of a cullin in the cells, and (c) administering the cells to the subject, thereby treating or preventing the neurodegenerative disorder.

In an aspect, provided herein is a method of treating or preventing a neurodegenerative disorder in a subject in need thereof, comprising: (a) obtaining a biological sample comprising cells from a subject suspected of having a neurodegenerative disorder; (b) contacting the cells with an effective amount of at least one agent which inhibits the level or activity of Nedd8 activating enzyme (NAE) in the cells, and (c) administering the cells to the subject, thereby treating or preventing the neurodegenerative disorder.

In some embodiments of this and any other aspect herein, the cells comprise neurons. In some embodiments of this and any other aspect herein, the cells comprise motor neurons. In some embodiments of this and any other aspect herein, the cells comprise non-neuronal cells. In some embodiments of this and any other aspect herein, the cells comprise somatic cells. In some embodiments of this and any other aspect herein, the cells comprise fibroblasts. In some embodiments of this and any other aspect herein, the cells are reprogrammed to induced pluripotent stem cells. In some embodiments of this and any other aspect herein, the cells are reprogrammed to neurons. In some embodiments of this and any other aspect, the cells are reprogrammed to motor neurons.

In some embodiments of this and any other aspect, the cells are expanded prior to administering the cells to the subject.

In some embodiments, the methods further comprise administering to the subject an effective amount of an agent or composition which regulates the overall level of SMN protein in the subject's cells. In some embodiments, the methods further comprise administering to the subject an effective amount of an agent or composition which inhibits ubiquitination of SMN protein. In some embodiments, the methods further comprise administering to the subject an effective amount of an agent or composition which modulates ubiquitin ligase activity of a cullin. In some embodiments, the methods further comprise administering to the subject an effective amount of an agent or composition which inhibits the level or activity of Nedd8 activating enzyme (NAE).

In an aspect, provided herein is a method for treating a neurodegenerative disorder in a subject, comprising administering a therapeutically effective amount of a cullin modulator to a subject in need thereof. Agents or compounds that modulate the activity of a cullin are also referred to as cullin modulators herein. As discussed above, cullins form the catalytic core of multisubunit ubiquitin ligases. Thus, activity of a cullin can be monitored by assaying the ubiquitin ligase activity of such a complex. Biochemical assays to monitor the ubiquitin ligase activities of cullins are known in the art. See for example, U.S. Pat. No. 6,413,725, content of which is incorporated herein by reference.

By “neurodegenerative disorder” is meant any disease or disorder caused by or associated with the deterioration of cells or tissues of the nervous system, exemplary neurodegenerative disorders are polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia. Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, peripheral neuropathy, Pick's disease, primary lateral sclerosis, Refsum's disease, Sandhoff disease, Schilder's disease, spinal cord injury, spinal muscular atrophy (SMA), SteeleRichardson-Olszewski disease, Tabes dorsalis, and traumatic brain injury.

The motor neuron diseases (MND) are a group of neurodegenerative disorders that selectively affect motor neurons, the nerve cells that control voluntary muscle activity including speaking, walking, breathing, swallowing and general movement of the body. Skeletal muscles are innervated by a group of neurons (lower motor neurons) located in the ventral horns of the spinal cord which project out the ventral roots to the muscle cells. These nerve cells are themselves innervated by the corticospinal tract or upper motor neurons that project from the motor cortex of the brain. On macroscopic pathology, there is a degeneration of the ventral horns of the spinal cord, as well as atrophy of the ventral roots. In the brain, atrophy may be present in the frontal and temporal lobes. On microscopic examination, neurons may show spongiosis, the presence of astrocytes, and a number of inclusions including characteristic “skein-like” inclusions, bunina bodies, and vacuolisation. Motor neuron diseases are varied and destructive in their effect. They commonly have distinctive differences in their origin and causation, but a similar result in their outcome for the patient: severe muscle weakness. Amyotrophic lateral sclerosis (ALS), primary lateral sclerosis (PLS), progressive muscular atrophy (PMA), pseudobulbar palsy, progressive bulbar palsy, spinal muscular atrophy (SMA) and post-polio syndrome are all examples of MND. The major site of motor neuron degeneration classifies the disorders. As used herein, the phrase “motor neuron degeneration” or “degeneration of motor neuron” means a condition of deterioration of motor neurons, wherein the neurons die or change to a lower or less functionally-active form.

Common MNDs include amyotrophic lateral sclerosis, which affects both upper and lower motor neurons. Progressive bulbar palsy affects the lower motor neurons of the brain stent, causing slurred speech and difficulty chewing and swallowing, individuals with these disorders almost always have abnormal signs in the arms and legs. Primary lateral sclerosis is a disease of the upper motor neurons, while progressive muscular atrophy affects only lower motor neurons in the spinal cord. Means for diagnosing MND are well known to those skilled in the art. Non limiting examples of symptoms are described below.

Spinal Muscular Atrophy (SMA): SMA refers to a number of different disorders, all having in common a genetic cause and the manifestation of weakness due to loss of the motor neurons of the spinal cord and brainstem. Weakness and wasting of the skeletal muscles is caused by progressive degeneration of the anterior horn cells of the spinal cord. This weakness is often more severe in the legs than in the arms, SMA has various forms, with different ages of onset, patterns of inheritance, and severity and progression of symptoms. Some of the more common SMAs are described below.

Delect in SMN gene products are considered as the major cause of SMA and SMN protein levels correlate with survival of subject suffering from SMA. The most common form of SMA is caused by mutation of the SMN gene. The region of chromosome 5 that contains the SMN (survival motor neuron) gene has a large duplication. A large sequence that contains several genes occurs twice in adjacent segments. There are thus two copies of the gene, SMN1 and SMN2. The SMN2 gene has an additional mutation that makes it less efficient at making protein, though it does so in a low level. SMA is caused by loss of the SMN1 gene from both chromosomes. The severity of SMA, ranging from SMA 1 to SMA 3, is partly related to how well the remaining SMN 2 genes can make up for the loss of SMN 1.

SMA type I, also called Werdnig-Hoffmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing. Some children also develop scoliosis (curvature of the spine) or other skeletal abnormalities. Affected children never sit or stand and the vast majority usually die of respiratory failure before the age of 2.

Symptoms of SMA type II usually begin after the child is 6 months of age. Features may include inability to stand or walk, respiratory problems, hypotonia, decreased or absent tendon reflexes, and fasciculations. These children may learn to sit but do not stand. Life expectancy varies, and some individuals live into adolescence or later.

Symptoms of SMA type III (Kugelberg-Welander disease) appear between 2 and 17 years of age and include abnormal gait; difficulty running, climbing steps, or rising from a chair; and a fine tremor, of the fingers. The lower extremities are was often affected. Complications include scoliosis and joint contractures—chronic shortening of muscles or tendons around joints, caused by abnormal muscle tone and weakness, which prevents the joints from moving freely.

Other forms of SMA include e.g., Hereditary Bulbo-Spinal SMA Kennedy's disease (X linked, Androgen receptor), SMA with Respiratory Distress (SMARD 1) (chromosome 11, IGHMBP2 gene). Distal SMA with upper limb predominance (chromosome 7, glycyl tRNA synthase), and X-Linked infantile SMA (gene UBE1)

Current treatment for SMA consists of prevention and management of the secondary effect of chronic motor unit loss. Some drugs under clinical investigation for the treatment of SMA include butyrates, Valproic acids, hydroxyurea and Riluzole.

Symptoms of Fazio-Londe disease appear between 1 and 12 years of age and may include facial weakness, dysphagia (difficulty swallowing), stridor (a high-pitched respiratory sound often associated with acute blockage of the larynx), difficulty speaking (dysarthria), and paralysis of the eye muscles. Most individuals with SMA type III die from breathing complications.

Kennedy disease, also known as progressive spinobulbar muscular atrophy, is an X-linked recessive disease. Daughters of individuals with Kennedy disease are carriers and have a 50 percent chance of having a son affected with the disease. Onset occurs between 15 and 60 years of age. Symptoms include weakness of the facial and tongue muscles, hand tremor, muscle cramps, dysphagia, dysarthria, and excessive development of male breasts and mammary glands. Weakness usually begins in the pelvis before spreading to the limbs. Some individuals develop noninsulin-dependent diabetes mellitus.

The course of the disorder varies but is generally slowly progressive. Individuals tend to remain ambulatory until late in the disease. The life expectancy for individuals with Kennedy disease is usually normal.

Congenital SMA with arthrogryposis (persistent contracture of joints with fixed abnormal posture of the limb) is a rare disorder. Manifestations include severe contractures, scoliosis, chest deformity, respiratory problems, unusually small jaws, and drooping of the upper eyelids.

Amyotrophic Lateral Sclerosis (ALS): ALS, also called Lou Gehrig's disease or classical motor neuron disease, is a progressive, ultimately fatal disorder that eventually disrupts signals to all voluntary muscles. In the United States, doctors use the terms motor neuron disease and ALS interchangeably. Both upper and lower motor neurons are affected. Approximately 75 percent of people with classic ALS will also develop weakness and wasting of the bulbar muscles (muscles that control speech, swallowing, and chewing). Symptoms are usually noticed first in the arms and hands, legs, or swallowing muscles. Muscle weakness and atrophy occur disproportionately on both sides of the body. Affected individuals lose strength and the ability to move their arms, legs, and body. Other symptoms include spasticity, exaggerated reflexes, muscle cramps, fasciculations, and increased problems with swallowing and forming words. Speech can become slurred or nasal. When muscles of the diaphragm and chest wall fail to function properly, individuals lose the ability to breathe without mechanical support. Although the disease does not usually impair a person's mind or personality, several recent studies suggest that some people with ACS may have alterations in cognitive functions such as problems with decision-making and memory. ALS most commonly strikes people between 40 and 60 years of age, but younger and older people also can develop the disease. Men are affected more often than women. Most cases of ALS occur sporadically, and family members of those individuals are not considered to be at increased risk for developing the disease. However, there is a familial form of ALS in adults, which often results from mutation of the superoxide dismutase gene, or SOD1, located on chromosome 21. In addition, a rare juvenile-onset form of ALS is genetic. Most individuals with ALS die from respiratory failure, usually within 3 to 5 years from the onset of symptoms. However, about 10 percent of affected individuals survive for 10 or more years.

Progressive bulbar palsy, also called progressive bulbar atrophy, involves the bulb-shaped brain stem—the region that controls lower motor neurons needed for swallowing, speaking, chewing, and other functions. Symptoms include pharyngeal muscle weakness (involved with swallowing), weak jaw and facial muscles, progressive loss of speech, and tongue muscle atrophy. Limb weakness with both lower and upper motor neuron signs is almost always evident but less prominent. Affected persons have outbursts of laughing or crying (called emotional lability), individuals eventually become unable to eat or speak and are at increased risk of choking and aspiration pneumonia, which is caused by the passage of liquids and food through the vocal folds and into the lower airways and lungs. Stroke and myasthenia gravis each have certain symptoms that are similar to those of progressive bulbar palsy and must be ruled out prior to diagnosing this disorder. In about 25 percent of ALS cases early symptoms begin with bulbar involvement. Some 75 percent of individuals with classic ALS eventually show some bulbar involvement. Many clinicians believe that progressive bulbar palsy by itself, without evidence of abnormalities in the arms or legs, is extremely rare.

Pseudobulbar palsy, winch shares many symptoms of progressive bulbar palsy, is characterized by upper motor neuron degeneration and progressive loss of the ability to speak, chew, and swallow. Progressive weakness in facial muscles leads to an expressionless face. Individuals may develop a gravelly voice and an increased gag reflex. The tongue may become immobile and unable in protrude from the mouth, individuals may also experience emotional lability.

Primary lateral sclerosis (PLS) affects only upper motor neurons and is nearly twice as common in men as in women. Onset generally occurs after age 50. The cause of PLS is unknown. It occurs when specific nerve cells in the cerebral cortex (the thin layer of cells covering the brain which is responsible for most higher level mental functions) that control voluntary movement gradually degenerate, causing the muscles under their control to weaken. The syndrome—which scientists believe is only rarely hereditary—progresses gradually over years or decades, leading to stiffness and clumsiness of the affected muscles. The disorder usually affects the legs first, followed by the body trunk, arms and hands, and, finally, the bulbar muscles. Symptoms may include difficulty with balance, weakness and stiffness in the legs, clumsiness, spasticity in the legs which produces slowness and stiffness of movement, dragging of the feet (leading to an inability to walk), and facial involvement resulting in dysarthria (poorly articulated speech). Major differences between ALS and PLS (considered a variant of ALS) are the motor neurons involved and the rate of disease progression. PLS may be mistaken for spastic paraplegia, a hereditary disorder of the upper motor neurons that causes spasticity in the legs and usually starts in adolescence. Most neurologists follow the affected individual's clinical course for at least 3 years before making a diagnosis of PLS. The disorder is not fatal bat may offset quality of life. PLS often develops into ALS.

Progressive muscular atrophy (PMA) is marked by slow but progressive degeneration of only the lower motor neurons. It largely affects men, with onset earlier than in other MNDs. Weakness is typically seen first in the hands and then spreads into the lower body, where it can be severe. Other symptoms may include muscle wasting, clumsy band movements, fasciculations, and muscle cramps. The trunk muscles and respiration may become affected. Exposure to cold can worsen symptoms. The disease develops into ALS in many instances.

Post-polio syndrome (PPS) is a condition that can strike polio survivors decades after their recovery from poliomyelitis. PPS is believed to occur when injury, illness (such as degenerative joint disease), weight gain, or the aging process damages or kills spinal cord motor neurons that remained functional after the initial polio attack. Many scientists believe PPS is latent weakness among muscles previously affected by poliomyelitis and not a new MND. Symptoms include fatigue, slowly progressive muscle weakness, muscle atrophy, fasciculations, cold intolerance, and muscle and joint pain. These symptoms appear most often among muscle groups affected by the initial disease. Other symptoms include skeletal deformities such as scoliosis and difficulty breathing, swallowing, or sleeping. Symptoms are more frequent among older people and those individuals most severely affected by the earlier disease. Some individuals experience only minor symptoms, while others develop SMA and, rarely, what appears to be, but is not, a form of ALS. PPS is not usually life threatening. Doctors estimate the incidence of PPS at about 25 to 50 percent of survivors of paralytic poliomyelitis.

In some embodiments, neurodegenerative disease is SMA. In some embodiments, neurodegenerative disease is ALS.

By “treatment, prevention or amelioration of neurodegenerative disorder” is meant delaying or preventing the onset of such a disorder (e.g. death of motor neurons), at reversing, alleviating, ameliorating, inhibiting, slowing down or stopping the progression, aggravation or deterioration the progression or severity of such a condition. In one embodiment, the symptom of a disorder characterized by degradation of SMN protein is alleviated by at least 20%, at least 30%, at least 40%, or at least 50%. In one embodiment, the symptom of a disorder characterized by degradation of SMN protein is alleviated by more than 50%. In one embodiment, the symptom of a disorder characterized by degradation of SMN protein is alleviated by 80%, 90%, or greater. In one embodiment, the symptom of a disorder characterized by diminished levels of SMN protein is alleviated by at least 20%, at least 30%, at least 40%, or at least 50%. In one embodiment, the symptom of a disorder characterized by diminished levels of SMN protein is alleviated by more than 50%. In one embodiment, the symptom of a disorder characterized by diminished levels of SMN protein is alleviated by 80%, 90%, or greater. In one embodiment, the symptom of a neurodegenerative disorder is alleviated by at least 20% at least 30%, at least 40%, or at least 50%. In one embodiment, the symptom of a neurodegenerative disease is alleviated by more than 50%. In one embodiment, the symptom of a neurodegenerative disorder is alleviated by 80%, 90%, or greater. In some embodiments, treatment also includes improvements in neuromuscular function. In some embodiments, neuromuscular function improves by at least about 10%, 20%, 30%, 40%, 50% or more. In some embodiments, treatment also includes increases in levels of SMN protein. In some embodiments, levels of SMN protein increases by at least about 10%, 20%, 30%, 40%, 50% or more. In some embodiments, treatment also includes a reduction in degradation of SMN protein. In some embodiments, degradation of SMN protein is reduced by at least about 10%, 20%, 30%, 40%, 50% or more. In some embodiments, treatment also includes a reduction in the number of dying cells (e.g., neurons, e.g., motor neurons). In some embodiments, the number of dying cells in a subject is reduced by at least about 10%, 20%, 30%, 40%, 50% or more. In some embodiments, treatment includes an increased number of surviving cells (e.g., neurons, e.g., motor neurons). In some embodiments, the number of surviving cells in a subject is increased by at least about 10%, 20%, 30%, 40%, 50% or more.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

In some embodiments, the subject is a human.

In some embodiments, the subject suffers from a disorder characterized by diminished levels of SMN protein. In some embodiments, the subject suffers from a disorder characterized by degradation of SMN protein. In some embodiments, the subject suffers from a neurodegenerative disease. In some embodiments, the neurodegenerative disease is characterized by diminished levels of SMN protein or degradation of SMN protein.

In some embodiments the methods described herein further comprise selecting a subject diagnosed with a disorder characterized by degradation of SMN protein. In some embodiments the methods described herein further comprise selecting a subject diagnosed with a disorder characterized by diminishes levels of SMN protein. In some embodiments the methods described herein further comprise selecting a subject diagnosed with a neurodegenerative disease. A subject suffering from a neurodegenerative disease can be selected based on the symptoms presented. For example a subject suffering from SMA may show symptoms of hypotonia, diminished limb movements, lack of tendon reflexes, fasciculations, tremors, swallowing, feeding difficulties, impaired breathing, scoliosis or other skeletal abnormalities, inability to stand or walk, abnormal gait, difficulty running, difficulty climbing steps, difficulty rising from a chair, and/or fine tremor of the fingers. In another example, a subject suffering from ALS may show symptoms of weakness and wasting of the bulbar muscles, such as difficulty speaking, swallowing, and chewing, muscle weakness and/or atrophy, decreased strength and reduced mobility, spasticity, exaggerated reflexes, muscle cramps, and fasciculations.

In some embodiments, the methods described herein further comprise diagnosing a subject for a neurodegenerative disease or disorder. In some embodiments, the methods described herein further comprise diagnosing a subject for a SMA. In some embodiments, the methods described herein further comprise diagnosing a subject for ALS.

In some embodiments, the method further comprises co-administering an additional pharmaceutically active agent approved for treatment of the neurodegenerative disorder or alleviating a symptom thereof.

In some embodiments, the method further comprises co-administering a second compound to the subject, wherein the second compound modulates a biological pathway or target other than a cullin, e.g., a biological pathway or target described herein.

### Compositions

The disclosure contemplates various compositions comprising one or more agents and/or compounds described herein, e.g., agents which block SMN ubiquitination, agents which inhibit the level or activity of NAE, agents which inhibit neddylation of a cullin, agents which increase the level of high SMN expressing cells (e.g., motor neurons) in a subject, agents which decrease the level of low SMN expressing cells (e.g., motor neurons) in a subject, agents which modulate the ligase activity of a cullin, etc.

In an aspect, provided herein is a composition, comprising/an effective amount of an agent which regulates the degradation of SMN protein.

In an aspect, provided herein is a composition, comprising an effective amount of an agent which increasing the level of SMN protein.

In an aspect, provided herein is a composition, comprising an effective amount, of a mutant cullin polypeptide lacking a neddylation site.

In an aspect, provided herein is a composition, comprising an effective amount of an agent which regulates the overall levels of SMN protein in a cell or subject.

In an aspect, provided herein is a composition, comprising an effective amount of an agent which inhibits SMN ubiquitination.

In an aspect, provided herein is a composition, comprising an effective amount of an agent which modulates ligase activity of a cullin.

In an aspect, provided herein is a composition, comprising an effective amount of an agent which inhibits the level or activity of NAE.

In an aspect, provided herein is a composition, comprising an effective amount of ML4924 or an analog or derivative thereof.

In an aspect, provided herein is a composition, comprising an effective amount an HDAC inhibitor.

In an aspect, provided herein is a composition, comprising an effective amount of an agent which selectively decreases the number of low SMN expressing cells in a subject.

In an aspect, provided herein is a composition comprising a therapeutically effective amount of Trichostatin A or an analog or derivative there.

In an aspect, provided herein is a composition comprising an effective amount of a compound of formula I.

In aspect, provided herein is a composition comprising an effective amount of an agent which increases the number of high SMN expressing cells in a subject, and an effective amount of an agent which decreases the number of low SMN expressing cells in a subject.

In an aspect, provided herein is a composition comprising an effective amount of a compound of formula I and an HDAC inhibitor.

In an aspect, provided herein is a composition comprising an effective amount of MLN4924 or an analog or derivative thereof and an effective amount of Trichostatin A or an analog or derivative thereof.

### Formulations and Administration

For administration to a subject, the compositions, agents and/or compounds e.g., agents which block SMN ubiquitination, agents which inhibit the level or activity of NAE, cullin modulators, etc., can be administered orally, parenterally, for example, subcutaneously, intravenously, intramuscularly, intraperitoneally, by intranasal instillation, or by application to mucous membranes, such as, that of the nose, throat, and bronchial tubes. One method for targeting the nervous system, such as spinal cord glia, is by intrathecal delivery. The targeted compound is released into the surrounding CSF and/or tissues and the released compound can penetrate into the spinal cord parenchyma, just after acute intrathecal injections. For a comprehensive review on drug delivery strategies including CNS delivery, see Ho et al., Curr. Opin. Mol. Ther. (1999), 1:336-3443, Groothuis et al., J. Neuro Virol. (1997), 3:387-400, and Jan. Drug Delivery SYstmes: Technologies and Commercial Opportunities, Decision Resources, 1998 and

They can be administered alone or with suitable pharmaceutical carriers, and can be in solid or liquid form such as, tablets, capsules, powders, solutions, suspensions, or emulsions.

As used herein, the term “administered” refers to the placement of an agent or compound described herein, into a subject by a method or route which results in at least partial localization of the agent or compound at a desired site. An agent or compound described herein can be administered by any appropriate route which results in effective treatment in the subject, i.e. administration results in delivery to a desired location in the subject where at least a portion of the agent or compound delivered. Exemplary modes of administration include, but are not limited to, injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The agents or compounds can be formulated in pharmaceutically acceptable compositions which comprise a therapeutically-effective amount of the agent or compound, formulated together with one or more pharmaceutically acceptable carriers (additives) and/or diluents. The agents or compounds can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol Toxicol. 24: 199-236 (1984); Lewis, ed. “Controlled Release of Pesticides and Pharmaceuticals” (Plenum Press, Hew York, 1981); U.S. Pat. No. 3,773,919; and U.S. Pat. No. 35 3,270,960.

As used here, the term “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve us pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methyl cellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

“PEG” means an ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG110000, PEG12000, PEG2000000 and any mixtures thereof.

The agents or compounds can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an agent or compound with one or more excipients or carriers. In general, the formulations are prepared by uniformly and intimately bringing into association one or more agents or compounds with liquid excipients or finely divided solid excipients or both, and then, if appropriate, shaping the product.

The preparative procedure may include the sterilization of the pharmaceutical preparations. The agents or compounds may be mixed with auxiliary agents such as lubricants, preservatives, stabilizers, salts for influencing osmotic pressure, etc., which do not react deleteriously with the agents or compounds.

Examples of injectable form include solutions, suspensions and emulsions. Injectable forms also include sterile powders for extemporaneous preparation of injectable solutions, suspensions or emulsions. The agents or compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatinA suitable carrier is 5% dextrose in saline. Frequently, it is desirable to include additives in the carrier such as buffers and preservatives or other substances to enhance isotonicity and chemical stability.

In some embodiments, agents or compounds can be administrated encapsulated within liposomes. The manufacture of such liposomes and insertion of molecules into such liposomes being well known its the art, for example, as described in U.S. Pat. No. 4,522,811. Liposomal suspensions (including liposomes targeted to particular cells, e.g., neurons) can also be used as pharmaceutically acceptable carriers. Methods of targeting agents or compounds to the CNS ere reviewed by Schnyder and Huwyler (NeuroRx. 2005; 2(1); 99-107).

In one embodiment, the agents or compounds are prepared with carriers that will protect the agent or compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polyacitic acid. Methods for preparation of such formulations will be apparent to those skilled in the art. The materials can also be obtained commercially from Alza Corporation and Nova Pharmaceuticals, Inc.

In the case of oral ingestion, excipients useful for preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like.

The agents or compounds can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these agents or compounds may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount or compound in such therapeutically useful compositions is such that a suitable dosage will be obtained. Preferred compositions according to the present invention are prepared so that an oral dosage unit contains between about 100 and 2000 mg of compound.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time alter the dose. Continuous infusion may also be used after the bolus dose.

The agents or compounds can also be administrated directly to the airways in the form of an aerosol. For administration by inhalation, the agents or compounds in solution or suspension can be delivered in the form of an aerosol spray from pressured container or dispenser which contains a suitable propellant, e.g., a gas such as carbon dioxide, or hydrocarbon propellant like propane, butane or isobutene. The agents or compounds can also be administrated in a no-pressurized form such as in an atomizer or nebulizer.

The agents or compounds can also be administered parenterally. Solutions or suspensions of these agents or compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of agent or compound calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the agents or compounds are formulated into ointments, salves, gels, or creams as generally known in the art.

The agents or compounds can be administrated to a subject in combination with a pharmaceutically active agent. Exemplary pharmaceutically active agent include, but are not limited to, those found in Harrison's Principles of Internal Medicine, 13th Edition, Eds. T. R. Harrison et al. McGraw-Hill N.Y., N.Y.; Physicians Desk Reference, 50^{th} Edition, 1997, Oradell, N.J., Medical Economics, Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference. In some embodiments, the pharmaceutically active agent is selected from the group consisting of butyrates, valproic acid, hydroxyuirae and Riluzole.

The agent or compound and the pharmaceutically active agent may be administrated to the subject in the same pharmaceutical composition or in different pharmaceutical compositions (at the same time or at different times). In some embodiments, the pharmaceutically active compound is an HDAC inhibitor (e.g., Trichostatin A).

The amount of agent or compound which can be combined with a carrier material to produce a single dosage form will generally be that amount of the agent or compound which produces a therapeutic effect. Generally out of one hundred percent, this amount will range from about 0.1% to 99% of compound, preferably from about 5% to about 70%, most preferably from 10% to about 30%.

The tablets, capsules, and the like may also contain a binder such as gum tragacanth, acacia, corn starch, or gelatin; excipients such as dicalcium phosphate; a disintegrating agent such as cornstarch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

The pharmaceutical compositions can be included in a container, pack, or dispenser together with instructions for administration. In some contexts herein, the term “therapeutically effective amount” means an amount of the agent or compound which is effective to promote the survival of cells (e.g., neurons) or to prevent or slow the death of such cells. In some contexts herein, the term “therapeutically effective amount” means an amount of the agent or compound which is effective to promote the survival of motor neuron cells or to prevent or slow the death of such cells. In some contexts herein, the term “therapeutically effective amount” means an amount of the agent or compound which is effective to reduce or prevent degradation of SMN protein in cells (e.g., neurons, e.g., motor neurons). In some contexts herein, the term “therapeutically effective amount” means an amount of the agent or compound which is elective to increase the levels of SMN protein in cells (e.g., neurons, e.g., motor neurons).

Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders.

Guidance regarding the efficacy and dosage which will deliver a therapeutically effective amount of an agent or compound to treat SMA can be obtained from animal models of SMA, see e.g., those described in Hsieb-Li et al. Nature Genetics. 2000; 24:66-70 and references cited therein.

Guidance regarding the efficacy and dosage which will deliver a therapeutically effective amount of an agent or compound to treat ALS can be obtained from animal models of SMA, e.g., a superoxide dismutase 1 (SOD1)(G93A) mutant mouse model of ALS or the wobbler mouse, see, e.g., Moser et al. The wobbler mouse, an ALS animal model, Mol Genet Genomics. 2013, DOI 10.1007/s00438-013-0741-0.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

The data obtained from the cell culture assays and animal studies can be used in formulating a range of dosage for use in humans. The dosage of such agents or compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, phosphorylation assays, NAE binding assays, and immunological assays.

The dosage may be determined by a physician and adjusted, as necessary, to suit observed effects of the treatment. Generally, the compositions are administered so that the agent or compound is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. For antibody agents or compounds, one preferred dosage is 0.1 mg/kg of body weight (generally 10 mg/kg to 20 mg/kg). If the antibody is to act in the brain, a dosage of 50 mg/kg to 100 mg/kg is usually appropriate.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. Examples of dosing schedules are administration once a week, twice a week, three times a week, daily, twice daily, three times daily or four or atom times daily.

### Survival of Motor Neuron (SMN)

Survival of Motor Neuron (SMN) is also known as component of gems 1 or Gemin-1. In human, there are two genes associated with this protein: SMN1 (also known as SMN or SMNT) and SMN2 (also known as SMNC).

The nucleotide and amino acid sequences of human SMN are disclosed in the art e.g., Lefebvre S. et al., Cell 80:155-165 (1995); Buerglen L. et al., Genomics 32:479-482 (1996); Chen Q. et al., Genomics 48:121-127 (1998); Gennarelli m. et al., Biochem. Biophys. Res. Commun. 213:342-348 (1995); Schmutz J. et al., Nature 431:268-274 (2004); The MGC Project Team, Genome Res. 14:2121-2127 (2004); and van der Steege G. et al., Eur. J. Hum. Genet. 3:87-95 (1995). The nucleotide and amino acid sequences of mouse SMN are disclosed in the art e.g., Viollet L. et al., Genomics 40:185-188 (1997); Didonato C. J. et al., Genome Res. 7:339-352 (1997); Schrank B. et al., Proc. Natl. Acad. Sci. U.S.A. 94:9920-9925 (1997); and the MGC Project Team; Genome Res. 14:2121-2127 (2004).

The SMN complex plays an essential role in spliceosomal snRNP assembly in the cytoplasm and is required for pre-mRNA splicing in the nucleus. It may also play a role in the metabolism of snoRNPs

The SMN protein is localized in the cytoplasm, nucleus and gem, ((subnuclear structures next to coiled bodies, called Gemini of Cajal bodies (Gems)).

The SMN protein is expressed in a wide variety of tissues. E.g., it is expressed at high levels in brain, kidney and liver, moderate levels in skeletal and cardiac muscle, and low levels in fibroblasts and lymphocytes. Its is also seen at high levels in spinal cord and is present in osteoclasts and mononuclear cells (at protein level).

The unprocessed human SMN protein is 294 amino acid in length with a molecular weight of about 32 kDa. The unprocessed mouse SMN protein is 288 amino acid in length with a molecular weight of about 31 kDa.

SMN is part of a stable complex that contains at least six other proteins, named Gemins 2-7, and is found in all metazoan cells. SMN protein is localized in the cytoplasm and in the nuclear structures called Gems that appear to be similar to and possibly interact with coded bodies. The full spectrum of SMN functions in nucleus and cytoplasm has not been determined. In in the cytoplasm, the SMN complex known to be a core mediator of assembly and trafficking of spliceosomal snRNP, and Gems are thought to be the centers of pre-mRNA splicing orchestrated by the SMN complex. It has been shown that cytosolic components phosphorylae SMN and transform the complex in to the active state. Interestingly, in both cases of overexpression of SMNΔexon7 and SMN+exon7, the number of nuclear gems was increased, however only the later construct promoted SMN increase in the cytoplasm. Although SMN protein is ubiquitously expressed, the main questions about why only motor neurons die during SMA, and which pool of SMN is essential for these cells are still remain. It has been presumed that disturbance in snRNP formation and splicing of motor neuron specific genes are attributers of such specificity. And indeed, it has been shown that ability of SMN complex to perform snRNP assembly determines the survival of animal with SMA (Workman E, HMG, 2009). In particular, there was noted a reduction of minor snRNP's that are responsible for splicing of approximately 700 genes (Gabanella F, PLoS, 2007). Second hypothesis points at the loss of function that SMN possibly has in motor neuronal axons. For example, reduced levels of β-actin mRNA transport to the growth cones after the number of certain Ca2+ channels (Rossoll W, JCB, 2003). Recently, cytoplasmic LSm4 protein known as part of the RNP complex associated with axonal RNA transport was shown to bind to SMN (Bealtie, 2008 review). Apart from splicing, SMN protein has also been reported to influence several other cellular activities such as transcription, ribosomal assembly, and apoptosis. Therefore SMN localization may reflect its multiple roles and their diversity is still a matter of further study.

SMA has recently attracted a great deal of attention from researchers because of its monogenic nature and seemingly straightforward path to the clinic. Data obtained from experiments on fibroblasts derived from SMA patients and from SMA mouse models suggest that therapeutics that elevate Survival of Motor Neuron (SMN) levels will be effective in treating this disease. Previously, large scale screens have been performed using libraries of diverse chemical structures with unknown biological activities in attempt to identify a scaffold that would be a clinical candidate for treating SMA disease. There are studies that describe screens based on the enzyme-reporter assays designed to identify compounds that stimulate Smn2 transcription or facilitate the splicing of Exon 7. It is well understood that reduced SMN levels is what ultimately causes SMA and these reporter assays only identify compounds that modulate the level of mRNA and are not designed to find comounds that elevane SMN protein, Alter SMN localization or stability. One of the promising high-throughput assays was established to study the mechanism of snRNP assembly and identified compounds that decrease the efficiency of this process; however no compounds were detected that promoted it (Dreyfuss, 2008). Several other screens were directed toward finding compoundws that increase the number of nuclear SMN-containing gems. However, since it has already been mentioned that the functional pools of SMN in motor effective compounds. Anothe rproblem with these assays is that the cells used have no obvious disease related phenotype. Therefore, this presents an additional challenge when trying to determine if compounds found in a primary screen can be effective in modifying the physiological defects that underlie SMA.

Results described herein show that there are several different classes of compounds that appear able to increase SMN. One of these pathways—PI-3 kinase, activated by different receptor tyrosine (RTK) ligands, is particularly effective. A downstream target of this pathway, GSK3 kinase, seems to be especially important. Inhibitors of this kinase elevate SMN levels in both patient fibroblasts and in motor neurons, derived from mouse embryonic stem cells. Importantly, they also decrease motor neuron death that follows reduction of SMN levels. Without wisthing to be bound by theory, it is believed that the agents which inhibit the level or activity of NAE exhibit additive effects with any agent that increases the levels of SMN protein (e.g., either full length or truncated SMN protein).

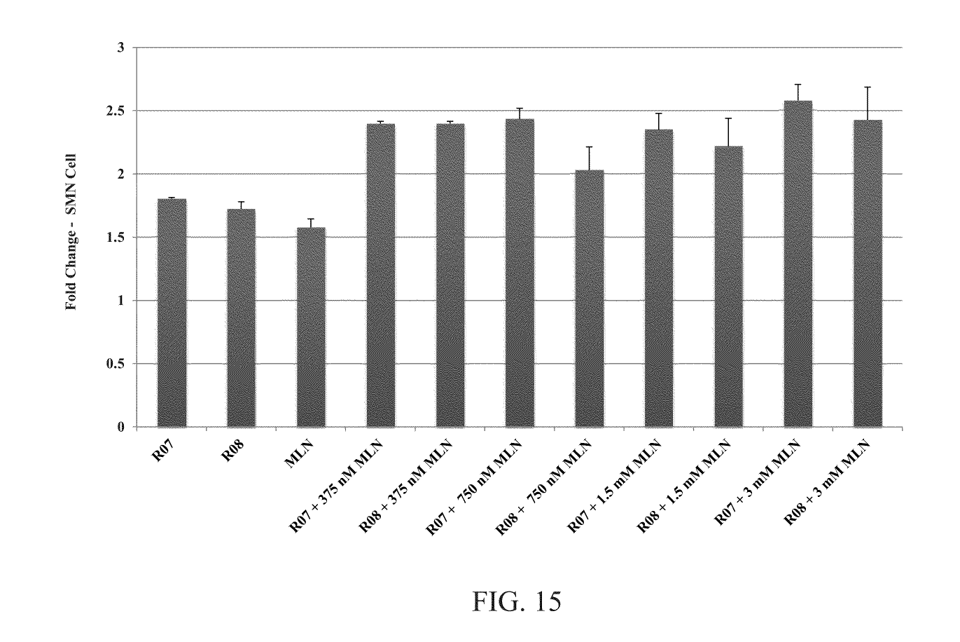

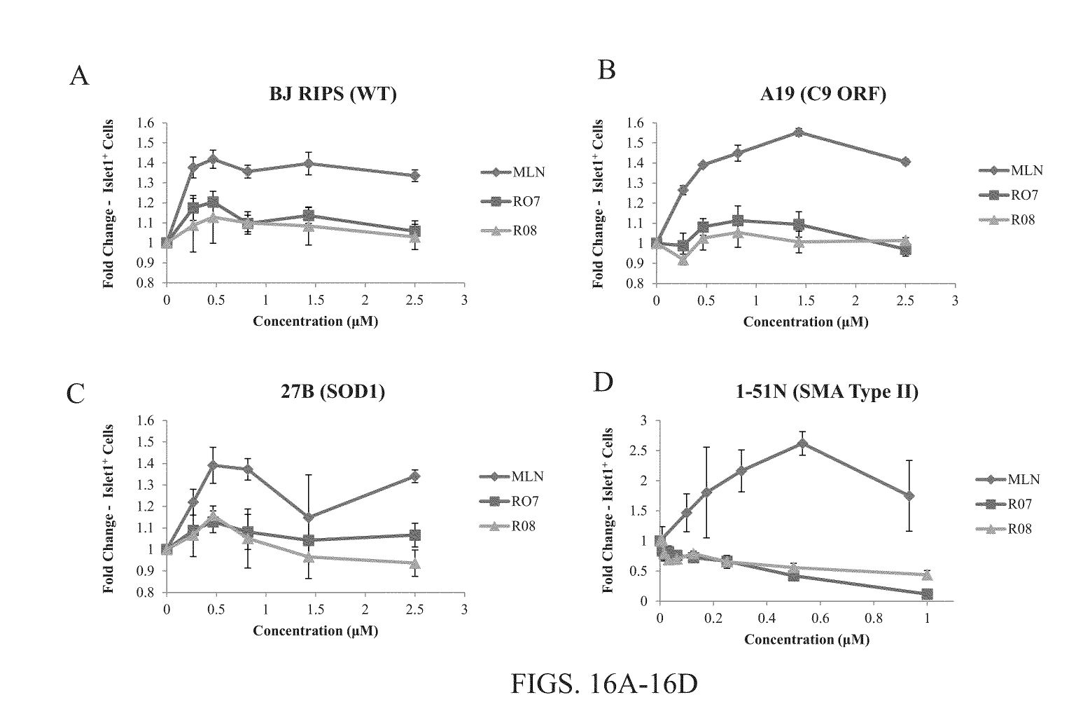

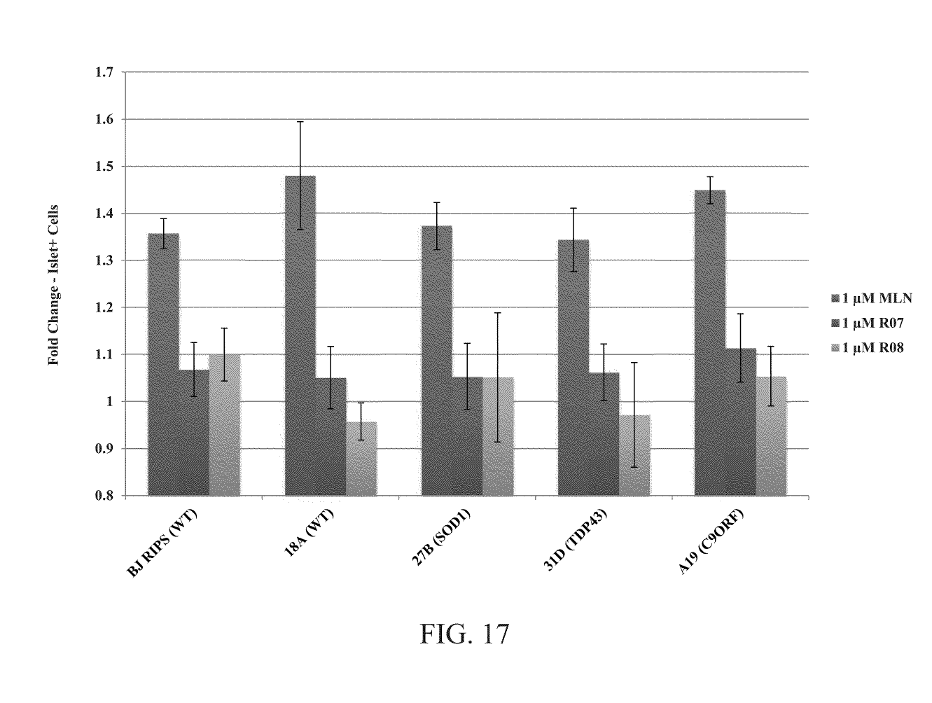

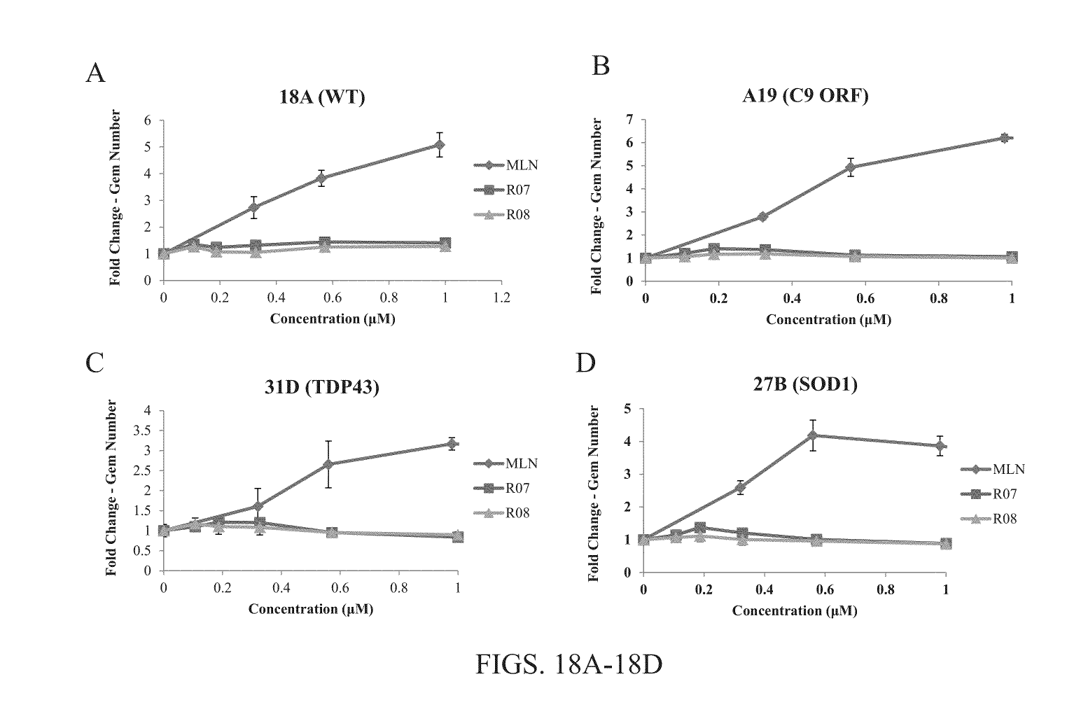

In some embodiments, an agent that increases the level of SMN protein comprises an SMN splicing modulator. As used herein, a “SMN splicing modulator” refers to an agent that increases the production of full-length SMN mRNA (e.g., SMN2 mRNA). SMN splicing modulators have been reported to improve motor function and longevity in mice with spinal muscular atrophy (see Naryshkin et al., “SMN2 splicing modifiers improve motor function and longevity in mice with spinal muscular atrophy”, Science, 345, 688 (2014). The data presented herein shows that Roche small molecule SMN splicing modulators R07 and R08 exhibit additive effects when used in combination with MLN4924 (FIG. 15). Without wishing to be bound by theory, it is believed that other SMN splicing modulators would exhibit similar additive effects when used in combination with MLN4924 and/or any other agent that inhibits the level and/or activity of NAE and/or agent that increases the level of SMN protein.

Examples of SMN splicing modulators include the orally bioavailable small organic molecule SMN splicing modulators (e.g., SMN-C1, SMN-C2 and SMN-C3) as described in Naryshkin et al, 2014.

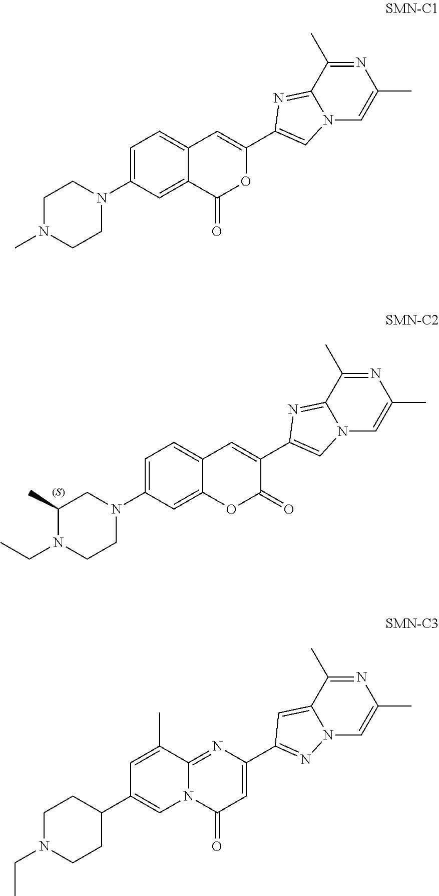

In some aspects, a SMN splicing modulator comprises an antisense oligonucleotide targeted to SMN2 pre-mRNA that induces SMN2 exon 7 inclusion and SMN protein expression. An example of such antisense oligonucleotide comprises 1S1S 396443, as described in Rigo et al., “Pharmacology of a central nervous system delivered 2′-O-methoxyethyl-modified survival of motor neuron splicing oligonucleotide in mice and nonhuman primates,” J Pharma Exp Ther., 350(1): 46-55 (2014). In some aspects, a SMN splicing modulator comprises a double-stranded RNA (e.g., siRNA) of between 15-30 bases that targets a splice junction or exonic or intronic sequence adjacent thereto in SMN2 pre-mRNA that produces an “exon-included” product, as described in U.S. Publication No. 2014/0128449. In some aspects, the double-stranded RNA contains one or more centrally located mismatches. In some aspects, the double-stranded RNA is fully complementary to a target site. Examples of such double-stranded sIRNAs targeting SMN2 pre-mRNA are shown in Table 1 below.

TABLE 1
siRNAs targeting SMN2 pre-mRNA

| | | Position of | |
|---|---|---|---|
| | | mismatch | Targeted |
| siRNA | Sequence and SEQ ID NO. | (if any) | region |
| | Fully complementary Duplexes | | |
| I82-I64FC | UAGCUUUAUAUGGAUGUUA (1) | — | ISS, intron 6 |
| I13-B06PC | UAAAACCCUGUAAGGAAAA (2) | — | Intron 6/exon 7 |
| I10-E09FC | UCUAAAACCCUGUAAGGAA (3) | — | Intron 6/exon 7 |
| I3-E16FC | GAUUUUGUCUAAAACCCUG (4) | — | ESS, exon 7 |
| E01-E19FC | UUUGAUUUUGUCUAAAACC (5) | — | ESS, exon 7 |
| I6-I24FC | CUUUCAUAAUGCUGGCAGA (6) | — | ISS, intron 7 |
| I9-I27FC | UCACUUUCAUAAUGCUGGC (7) | — | ISS, intron 7 |
| I86-I104FC | CUUUCUAACAUCUGAACUU (8) | — | ISS, intron 7 |
| I89-I107FC | CAACUUUCUAACAUCUGAA (9) | — | ISS, intron 7 |
| | Mismatch-containing duplexes | | |
| I82-I64 | UAGCUUUAUUUGGAUGUUA (10) | 10 | ISS, intron 6 |
| I1112-I94 | GUUUCACAACACAUUUUAC (11) | 10 | ISS, intron 6 |
| I5-E14 | UUUUGUCUAAUACCCUGUA (12) | 11 | ESS, exon 7 |
| I3-E16 | GAUUUUGUCAAAAACCCUG (13) | 10 | ESS, exon 7 |
| E01-E19 | UUUGAUUUUGACUAAAACC (14) | 11 | ESS, exon 7 |
| E25-E42 | AGGAAUGUGACCACCUUCC (15) | 11 | Exon 7 |
| I6-I24 | CUUUCAUAAAGCUGGCAGA (16) | 10 | ISS, intron 7 |
| I9-I27 | UCACUUUCAAAAUGCUGGC (17) | 10 | ISS, intron 7 |
| I86-I104 | CUUUCUAACUUCUGAACUU (18) | 10 | ISS, intron 7 |
| | Noncomplementary duplex | | |
| CM | GCUAUACCAGCGUCGUCAU (19) | | |

The data presented herein also shows that MLN4924 (also referred to herein as MLN) increases SMN and promote motor neuron survival in human motor neurons. As shown herein, MLN increases SMN in human MNs, both in measures of average SMN and # of high SMN expressors. While, preliminary results of MLN4924 on MN survival appear to be variable, it is believed that the varying effects on MN survival can depend on the amount of proliferation in the culture since MLN4924 inhibits proliferation. The effect on proliferation can be dissociated, for example, by using AraC in protocols for differentiation of cells into motor neurons. The data presented herein also shows that MLN promotes survival of motor neurons derived from ALS patients and their parents, as well as motor neurons derived from SMA patterns and their parents.

### Biological Pathways and Targets

Non-limiting examples of the biological pathways include e.g. PI3K signaling pathway, Akt signaling pathway, MAPK signaling pathway, PDGF pathway, RAS pathway, elF2 pathway, GSK pathway, PKR pathway, Insulin Receptor Pathway, mTOR pathway, EGF pathway, NGF pathway, FGF pathway and BMP/TGFβ pathway. Non-limiting examples of the targets that modulate levels of SMN include e.g. components of the biological pathways described, herein. In some preferred embodiments, the targets that modulate levels of SMN include e.g., Na+/K+ channel, MAPK, cannobinoid receptor, GPCR, CA2+ channel, K+ channel, PDE5, GSK/CDK, GSK, PKR, CDK2, IKK-2, HDAC, proteasome, BMP/TGFβ receptor and Dopamine receptor.

PI3K signaling pathway: The definition and details of the PI3K signaling pathway are disclosed in the art e.g., Abell K. and Watson, C. J. Cell Cycle 4, 897-900 (2005); Brachmann, S. M. et al., Mol. Cell Biol. 25, 2593-2606 (2005); Katso R. et al., Annu. Rev. Cell Dev. Biol. 17, 615-675 (2001); and Vanhaesebroeck B. and Waterfield M. D. Exp. Cell Res. 253, 239-254 (1999).

Akt signaling pathway: The definition and details of the PI3K signaling pathway are disclosed in the art e.g., Downward, J. Curr. Opin. Cell Biol. 10, 262-267 (1988); Jimenez, C. et al., J. Biol. Chem. 277(404):41556-41562 (2002); Kitamura, T. et al., Mol. Cell Biol. 19, 6286-6296 (1999); Ruggero D. and Sonenberg n. Oncogene, 24, 7426-34 (2005); Testa J. R. and Tsichlis P. N. Oncogene, 7391-7393 (2005); and Zhou X. M. et al., J. Biol. Chem. 275,25046-25051 (2000).

MAPK signaling pathway: The definition and details of the MAPK signaling pathway are disclosed in the art e.g., Ichijo H. et al., Science 275, 90-94 (1997); Qiu M. S. and Green S. H. Neuron 7, 937-946 (1991); Rubinfeld H. and Seger R. Mol. Biotechnol. 31, 151-174 (2005); and Yoon S. and Seger R. Growth Factors, 24, 21-44 (2006).

PDGF pathway: The definition and details of the PDGF pathway are disclosed in the art e.g., Fredriksson L. et al., J. Biol. Chem. 280, 26856-26862 (2005); Hauck C. R. et al., J. Biol. Chem. 275, 41092-41099 (2000); Hoch R. V. and Soriano P. Development 130, 4769-4784 (2003); Jiang B. et al., Surgery, 120, 427-431 (1996); and Relgstad L. J. et al., FEBS J. 272, 5723-5741 (2005).

RAS pathway: The definition and details of the RAS pathway are disclosed in the art e.g., Ada-Nguema A. S. et al. J. Cell Sci. 119, 1307-1319 (2006); Hofer F. et al., Pro. Natl. Acad. Sci. 91, 11089-11093 (1994); Kikuchi A. et al. Mol. Cell. Biol. 14, 7483-7491 (1994); Rodriguez-Viciana, P. et al., Nature 370, 527-532; and Rubio, I. et al., Biochem. J. 326, 891-895 (1997).

eIF2 pathway: The definition and details of the eIF2 pathway are disclosed in the art e.g., Clemens J. J. pi Prog. Mol. Subcell. Biol. 27, 57-89 (2001); Proud C. G. Semin. Cell Dev. Biol. 16, 3-12 (2005); and Wek R. C. et al., Biochem. Soc. Trans. 34, 7-11 (2006).

GSK3 pathway: The definition and details of the GSK3 pathway are disclosed in the art e.g., Biondi R. M. and Nebreda A. R. Biochem J. 372, 1-13 (2003); Jope R. S. and Johnson G. V. Trends Biochem Sci. 29, 95-102 (2004); and Polakis P. Curr. Biol. 12, R499-R501 (2002).

PKR pathway: The definition and details of the PKR pathway are disclosed in the art e.g., Bennett R. L. et al., Blood, 108, 821-829 (2006); Donze O. et al., EMBO J. 23, 564-571 (2004); Guerra S. et al., J. Biol. Chem. 281, 18734-18745 (2006); and Li S. et al., Proc. Natl. Acad. Sci. U S A. 103, 10005-10010 (2006).

Insulin Receptor Pathway: The definition and details of the Insulin Receptor pathway are disclosed in the art e.g., Dudek H. et al., Science, 275, 661-665 (1997); Pandini G. et al., J. Biol Chem. 277, 39684-39695 (2002); and White M. F. and Myeres M. G. In Endocrinology (DeGroot, L. J., and Jameson, J. L., eds)., W. B. Saunders Co., Philadelphia (2001).

mTOR pathway: The definition and details of the mTOR pathway are disclosed in the art e.g., Gingras A. C. et al., Genes Dev. 15, 807-826 (2001); Hannan K. M. et al., Mol. Cell Biol. 23, 8862-8877 (2003); Kim D. H. et al., Cell 110, 163-175 (2002); Kumar V. et al., J. Biol. Chem. 275, 10779-10787 (2000); and Raught B. et al., Proc. Natl. Acad. Sci. USA 98, 7037-7044 (2001).

EGF pathway: The definition and details of the EGF pathway are disclosed in the art e.g., Carpenter G. and Ji Q, Exp. Cell Res. 253, 15-24 (1999); Garcia R. et al., Oncogene 20, 2499-2513 (2001); Henson E. S. and Gibson S. B. Cell Signal, (2006); Olayioye M. A. et al., J. Biol. Chem. 275, 17209-17218 (1999); Guren T. K. et al. J. Cell Physiol. 196, 113-125 (2003); and Sato K. et al., J. Biol. Chem. 277, 29568-29576 (2002).

NGF pathway: The definition and details of the EGF pathway are disclosed in the art e.g., Coulson E. J. Prog. Brain Res. 146, 41-62 (2004); Huang E. J. and Reichardt L. F. Annu. Rev. Biochem. 72, 609-642 (2003); Miller F. D. and Kaplan D. R. Cell Mol. Life Sci. 58, 1045-1053 (2001); and Rabizadeh S. and Bredesen D. E. Cytokine Growth Factor Rev. 14, 225-239 (2003).

FGF pathway: The definition and details of the FGF pathway are disclosed in the art e.g., Lee P. L. et al., Science, 245, 57-60 (1989); Mignatti P. et al., J. Cell Physiol. 151, 81-93 91992); Miki T. et al., Proc. Natl. Acad. Sci. USA. 89, 246-250 (1992); Gringel S. et al., J. Biol. Chem. 385, 1203-1208 (2004); and Ornitz D. M. and Itoh, N. Genome Biol. 2, 1-12 (2001); Sorensen V. et al., Bioessays. 28, 504-514 (2006).

BMP/TGFβ pathway: The definition and details of the BMP/TGFβ pathway are disclosed in the art e.g., Kawabata M. and Miyazono K., J. Biochem. (Tokyo), 125, 9-16 (1999); Wrana J. L., Miner. Electrolyte Metab., 24, 120-130 (1998); and Markowitz S. D., and Roberts A. B., cytokine Growth Factor Rev., 7, 93-102 (1996).

Phosphoinositide 3-kinases: phosphoinositide 3-kinases (PI 3-kinases or PI3Ks) are a family of related enzymes that are capable of phosphorylating the 3 position hydroxyl group of the inositol ring of phosphatidylinositol (Ptdlns), Ptdlns are described on the web at en.wikipedia.org/wiki/Phosphoinositide_{—}3-kinase—cite_note-0. They are also known as phosphatidylinositol-3-kinases.

PI3Ks interact with the IRS (Insulin receptor substrate) in order to regulate glucose uptake through a series of phosphorylation events. The phosphoinositol-3-kinase family is composed of Class I, II and Class III, with Class I the only ones able to convert PI(4,5)P2 to PI(3,4,5)P3 on the inner leaflet of the plasma membrane.

Class I PI3K are heterodimeric molecules composed of a regulatory and a catalytic subunit; they are further divided between IA and IB subsets on sequence similarity. Class IA PI3K are composed of one of five regulatory p85α, p55α, p50α, p85β or p55γ subunit attached to a p110α, β or δ catalytic subunit. The first three regulatory subunits are all splice variants of the same gene (Pik3r1), the other two being expressed by other genes (Pik3r2 and Pik3r3, p85β and p55γ, respectively). The most highly expressed regulatory subunit is p85α, all three catalytic subunits are expressed by separate genes (Pik3ca, Pik3cb and Pik3cd for p110α, 110β and p110δ, respectively). The first two p110 isoforms (α and β) are expressed in all cells, but p110δ is primarily expressed in leukocytes and it has been suggested it evolved in parallel with the adaptive immune system. The regulatory p101 and catalytic p110γ subunits comprise the type IB PI3K and are encoded by a single gene each.

Class II comprises three catalytic isoforms (C2α, C2β, and C2γ), but unlike Classes I and III, no regulatory proteins. These enzymes catalyse the production of PI(3)P from PI (may also produce PI(3,4)P2 from PI(4)P). C2α and C2β are expressed throughout the body, however expression of C2γ is limited to hepatocytes. The distinct feature of Class II PI3Ks is the C-terminal C2 domain. This domain lacks critical Asp residues to coordinate binding of Ca²⁺, which suggests class II PI3Ks bind lipids in a Ca²⁺ independent manner.

class III are similar to II in that they bias the production of PI(3)P from PI, but are more similar to Class I in structure, as they exist as a heterodimers of a catalytic (Vps34) and a regulatory (p150) subunits. Class III seems to be primarily involved in the trafficking of proteins and vesicles.

All PI 3-kinases are inhibited by the drugs wortmannin and LY294002, although certain member of the class II PI 3-kinase family show decreased sensitivity.

PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110α is mutated in many cancers. The PtdIns(3,4,5)P3 phosphatase PTEN which antagonises PI 3-kinase signalling is absent from many tumors. Hence, PI 3-kinase activity contributes significantly to cellular transformation and the development of cancer. The p110δ and p110γ isoforms regulate different aspects of immune responses. PI 3-kinases are also a key component of the insulin signaling pathway.

AKT is activated as a result of PI3-kinase activity, because AKT requires the formation of the PtdIns(3,4,5)P3 (or “PIP3”) molecule in order to be translocated to the cell membrane. At PIP3, AKT is then phosphorylated by phosphoinositide dependent kinase 1 (PDK1), and is thereby activated. The “PI3-k/AKT” signaling pathwas has been shown to be required for an extremely diverse array of cellular activities such as cellular proliferation and survival.

In addition to AKT and PDK1, one other related serine threonine kinase is bound at the PIP3 molecule created as a result of PI3-kinase activity, SGK.

PI3K has also been implicated in Long term potentiation (LTP).

The PI3K pathway also recruits many other proteins downstream, including mTOR, GSK3β, and PSD-95. The PI3K-mTOR pathway leads to the phosphorylation of p70S6K, a kinase which facilitates translational activity.

Glycogen synthase kinase 3 (GSK-3): Glycogen synthase kinase 3 (GSK-3) is a serine/threonine protein kinase. In mammals GSK-3 is encoded by two known genes GSK-3α. See description on the web at en.wikipedia.org/wiki/GSK3A and β. The nucleotide and amino acid sequences of human GSK-3α are disclosed in the art e.g., Hoshino T. et al., “Isolation of cDNA clones for human glycogen synthase kinase 3alpha.”, Submitted (NOV-1997) to the eMBL/GenBank/DDBJ databases; Grimwood J., et al., Nature 428:529-535 (2004); and The MGC Project Team, Genome Res. 14:2121-2127 (2004). The nucleotide and amino acid sequences of human GSKk-3β are disclosed in the art e.g., Stambolic V. and Woodgett J. R. Biochem. J. 303:701-704 (1994); The MGC Project Team, Genome Res. 14:2121-2127 (2004); Rhoads A. R. et al., Mol. Psychiatry 4:437-442 (1990); and Lau K. F. et al., Genomics 60:121-128 (1999).

GSK-3α is implicated in the hormonal control of several regulatory proteins including glycogen synthase, MYB and the transcription factor JUN, GSK-3β participates in the Wnt signaling pathway. It is implicated in the hormonal control of several regulatory proteins including glycogen synthase, MYB and the transcription factor JUN. It also phosphorylates JUN at sites proximal to its DNA-binding domain, thereby reducing its affinity for DNA. It phosphorylates MUC1 in breast cancer cells, and decreases the interaction of MUC1 with CTNNB1/beta-catenin. GSK-3β is inhibited when phosphorylated by AKT1.

GSK-3β is expressed in testis, thymus, prostate and ovary and weakly expressed in lung, brain and kidney.

The unprocessed human GSK-3α protein is 483 amino acid in length with a molecular weight of about 51 kDa. The unprocessed human GSK-3β protein is 420 amino acid in length with a molecular weight of about 47 kDa.

Ca2+ channel: A Calcium channel is an ion channel which displays selective permeability to calcium ions. It is also called as voltage-dependent calcium channel, although there are also ligand-gated calcium channels.

Calcium channel blockers are a class of drugs and natural substances with effects on many excitable cells of the body such as the muscle of the heart, smooth muscles of the vessels or neuron cells. Classes of calcium channel blockers include e.g., Dihydropyridine, Phenylalkylamine, Benzothiazepine.

cGMP-specific 3′,5′-cyclic phosphodiesterase (PDE5): PDE5 refers to a cGMP-binding, cGMP-specific phosphodiesterase, a member of the cyclic nucleotide phosphodiesterase family. This phosphodiesterase specifically hydrolyzes cGMP to 5′-GMP. It is involved in the regulation of intracellular concentrations of cyclic nucleotides and is important for smooth muscle relaxation in the cardiovascular system. Human PDE5 is expressed in aortic smooth muscle cells, heart, placenta, skeletal muscle and pancreas and, to a much lesser extent, in brain, liver and lung.

A PDE5 inhibitor, is a drug used to block the degradative action of PDB5 on cyclic GMP in the smooth muscle cells lining the blood vessels supplying the corpus cavernosum of the penis. These drugs are used in the treatment of erectile dysfunction. Because PDE5 is also present in the arterial wall smooth muscle within the lungs, PDE5 inhibitors have also been explored for the treatment of pulmonary hypertension, a disease in which blood vessels in the lungs become abnormally narrow.

Cannabinoid Receptors: The cannabinoid receptors refer to members of the family of guanine-nucleotide-binding protein (G-protein) coupled receptors which inhibit adenylate cyclase activity in a dose-dependent, stereoselective and pertussis toxin-sensitive manner. The cannabinoid receptors have been found to be involved in the cannabinoid-induced CNS effects (including alterations in mood and cognition) experienced by users of marijuana. Their ligands are known as cannabinoids or endocannabinoids.

Histone Deacetylase (HDAC): Histone deacetylases (HDAC) are a class of enzymes that remove acetyl groups from an ε-N-acetyl lysine amino acid on a histone. Exemplary HDACs include those Class I HDAC: HDAC1, HDAC2, HDAC3, HDAC8; and Class II HDACs: HDAC4, HDAC5, HDAC6, HDAC7A, HDAC9, HDAC10. Type I mammalian HDACs include: HDAC1, HDAC2, HDAC3, HDAC8, and HDAC11. Type II mammalian HDACs include: HDAC4, HDAC5, HDAC6, HDAC7, HDAC9, and HDAC10.

Cardiac Glycosides: Cardiac glycosides are drugs used in the treatment of congestive heart failure and cardiac arrhythmia. Cardiac glycosides work by inhibiting the Na⁺/K⁺ pump. This causes an increase in the level of sodium ions in the myocytes, which then leads to a rise in the level of calcium sons. This inhibition increases the amount of Ca²⁺ ions available for contraction of the heart muscle, improves cardiac output and reduces distention of the heart.

Inhibitor of IκB kinase 2 (IKK2): IKK2 is a protein which is a component of a cytokine-activated intracellular pathway involved in triggering immune responses. Activation of IKK2 leads to phosphorylation of the inhibitor of Nuclear Transcription factor kappa-8 (IκB). Phosphorylation of IκB causes the degradation of the inhibitor IκB via the ubiquination pathway, thereby allowing the transcription factor NFκB to enter the cell's nucleus and activate various genes involved in inflammation and other immune responses.

IKK2 plays a significant factor in the state of brain cells after a stroke. Experimental mice that had an overactive form of IKK2 experienced the loss of many more neurons than controls did after a stroke-simulating event.

Cyclin-dependent kinase 2 (CDK2): The protein encoded by this gene is a member of the cyclin-dependent kinase family of Ser/Thr protein kinases. This protein kinase is highly similar to the gene products of S. cerevisiae cde28, and S. pombe cdc2. It is a catalytic subunit of the cyclin-dependent kinase complex, whose activity is restricted to the G1-S phase of the cell cycle, and is essential for the G1/S transition. This protein associates with and regulated by the regulatory subunits of the complex including cyclin E or A. Cyclin E binds G1 phase Cdk2, which is required for the transition from G1 to S phase while binding with Cyclin A is required to progress through the S phase. Its activity is also regulated by phosphorylation. Two alternatively spliced variants and multiple transcription initiation sites of this gene have been reported. The role of this protein in GAS transition has been recently questioned as cells lacking Cdk2 are reported to have no problem during this transition.

Known CDK inhibitors are p21C1p1 (CDKN1A) and p27Kip1 (CDKN1B). Drugs which inhibit Cdk2 and arrest the cell cycle may reduce the sensitivity of the epithelium to many cell cycle-active antitumor agents end therefore represent a strategy for prevention of chemotherapy-induced alopecia.

### Kits

In another aspect, provided herein is a kit. In some aspects, a kit comprises (a) an agent which regulates degradation SMN protein; and (b) informational material. In some aspects, a kit comprises (b) an agent which increases the level of SMN protein; and (b) informational material. In some aspects, a kit comprises (a) an agent which regulates the overall level of SMN protein; and (b) informational material. In some embodiments of any aspect described herein, informational material comprises Instructions for using the agent for promoting motor neuron survival. In some embodiments, regulating the overall level of SMN protein promotes motor survival by at least one of increasing the levels of SMN protein, stabilizing levels of SMN protein, reducing degradation of SMN protein, preventing degradation of SMN protein, increasing the number of high SMN expressing cells, stabilizing the number of high SMN expressing cells, increasing the ratio of high SMN expressing cells to low SMN expressing cells or decreasing the number of low SMN expressing cells.

In an aspect, a kit comprises: (a) an agent which inhibits SMN ubiquitination; and (b) informational material (e.g., instructions for using the agent for promoting motor neuron survival). In an aspect, a kit comprises: (a) an agent which modulates ligase activity of a cullin; and (b) informational material (e.g., instructions for using the agent for promoting motor neuron survival). In an aspect, a kit comprises (a) an agent which inhibits the level or activity of NAE; and (b) informational material (e.g., instructions for using the agent for promoting motor neuron survival).

A kit comprising: (a) an agent which regulates the overall level of SMN protein; and (b) instructions for using the agent for treating or preventing a neurodegenerative disorder wherein regulating the overall level of SMN protein treats or prevents a neurodegenerative disorder by at least one of increasing the levels of SMN protein, stabilizing levels of SMN protein, reducing degradation of SMN protein, preventing degradation of SMN protein, increasing the number of high SMN expressing cells, stabilizing the number of high SMN expressing cells, increasing the ratio of high SMN expressing cells to low SMN expressing cells, or decreasing the number of low SMN expressing cells.

In an aspect, a kit comprises: (a) an agent which inhibits SMN ubiquitination; and (b) informational material (e.g., instructions for using the agent for treating or preventing a neurodegenerative disorder).

In an aspect, a kit comprises: (a) an agent which modulates ligase activity of a cullin; and (b) informational material (e.g., instructions for using the agent for treating or preventing a neurodegenerative disorder).

In an aspect, a kit comprises; (a) an agent which inhibits the level or activity of NAE; and (b) informational material (e.g., instructions for using the agent for treating or preventing a neurodegenerative disorder).

In some embodiments, the kit comprises (a) an agent or compound, e.g., a cullin modulator, and (b) informational material. The informational material can be descriptive, instructional, marketing or other material that relates to the methods described herein and/or the use of the agent or compound for the methods described herein. For example, the informational material describes methods for administering the agent or compound to alter lifespan regulation or at least one symptom of aging or an age related disease.

In some embodiments the informational material describes instructions to treat a neurodegenerative disorder e.g. SMA or ALS using a method described herein.

In some embodiments, the informational material can include instructions to administer an agent or compound in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., an adult human. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

So addition to the agent or compound, the composition of the kit can include other ingredients, such as a solvent or buffer, a stabilizer or a preservative, and/or a second agent for treating a condition or disorder described herein, e.g. increased pancreatic islet mass. Alternatively, the other ingredients can be included in the kit, but in different compositions or containers than the agent or compound. In such embodiments, the kit can include instructions for admixing the agent or compound and the other ingredients, or for using the modulator together with the other ingredients.

The agent or compound can be provided in any form, e.g., liquid, dried or lyophilized form. It is preferred that the agent or compound be substantially pure and/or sterile. When the agent or compound is provided in a liquid solution, the liquid solution preferably is an aqueous solution, with a sterile aqueous solution being preferred. When the agent or compound is provided as a dried form, reconstitution generally is by the addition of a suitable solvent. The solvent, e.g., sterile water or buffets can optionally be provided in the kit.

The kit can include one or more containers for the composition containing the agent or compound. In some embodiments, the kit contains separate containers, dividers or compartments for the agent or compound (e.g., in a composition) and informational material. For example, the agent or compound (e.g., in a composition) can be contained in a bottle, vial, or syringe, and the informational material can be contained in a plastic sleeve or packet. In other embodiments, the separate elements of the kit are contained within a single, undivided container. For example, the agent or compound (e.g., in a composition) is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agent or compound (e.g., in a composition). For example, the kit includes a plurality of syringes, ampules, foil packets, or blister packs, each containing a single unit dose of the agent or compound. The containers of the kits can be air tight and/or waterproof.

### Some Selected Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compounds, compositions, methods, and respective component(s) thereof that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than the operating examples, or where otherwise indicated, all numbers expressing quantities, of ingredients or reaction conditions used herein, should be understood as modified in all distances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the term “modulate” means to cause or facilitate a qualitative or quantitative change, alteration, or modification in a molecule, a process, pathway, or phenomenon of interest. Without limitation, such change may be an increase, decrease, a change in binding characteristics, or change in relative strength or activity of different components or branches of the process, pathway, or phenomenon. The term “modulator” refers to any molecule or compound that causes or meditates a qualitative or quantitative change, alteration, or modification in a process, pathway, or phenomenon of interest.

As used herein, the phrase “modulation of a biological pathway” refers to modulation of activity of at least one component of the biological pathway. It is contemplated herein that modulator of the signaling pathway can be, for example, a receptor ligand (e.g., a small molecule, an antibody, an siRNA), a ligand sequestrant (e.g., an antibody, a binding protein), a modulator of phosphorylation of a pathway component or a combination of such modulators.

One of skill in the art can easily test a compound to determine if it modulates a signaling pathway by assessing, for example, phosphorylation status of the receptor or expression of downstream proteins controlled by the pathway in cultured cells and comparing the results to cells not treated with a modulator. A modulator is determined to be a signaling pathway modulator if the level of phosphorylation of the receptor or expression of downstream proteins in a culture of cells is reduced by at least 20% compared to the level of phosphorylation of the receptor or expression of downstream proteins in cell that are cultured in the absence of the modulator; preferably the level of phosphorylation is altered by at least 30%, at least 40%, at least 50%, at least 60%, at least 70%, at least 80%, at least 90%, at least 95%, or at least 99% in the presence of a pathway modulator.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used Herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including, a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “small molecule” can refer to compounds that are “natural product-like,” however, the term, “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some embodiments, a small molecule can have a molecular weight equal to or less than 700 Daltons. In some embodiments, a small molecule can have a molecular weight equal to or less than 500 Daltons.

As used herein, an “RNA interference molecule” refers to a compound which interferes with or inhibits expression of a target gene or genomic sequence by RNA interference (RNAi). Such RNA interfering agents include, but are not limited to nucleic acid molecules including RNA molecules which are homologous to the target gene or genomic sequence, or a fragment thereof, short interfering RNA (siRNA), short hairpin or small hairpin RNA (shRNA), microRNA (mRNA) and small molecules which interfere with or inhibit expression of a target gene by RNA interference (RNAi).

The term “polynucleotide” is used herein interchangeably with “Nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. the succession of letters used as abbreviations for bases) that biochemically characterizes a specific nucleic acid. A polynucleotide sequence presented herein is presented in a 5′ to 3′ direction unless otherwise indicated.

The nucleic acid molecules that modulate the biological pathways or targets described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical preparation of the gene therapy vector can include the same therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc . . . A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein, is presented in an N-terminal to C-terminal direction unless otherwise indicated.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent ideality can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol, 215:403-410, 1990). To obtain gapped alignments for comparison purposes. Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nib.gov for these programs. In a specific embodiment, percent identity is calculated using BLAST2 with default parameters as provided by the NCBI.

The term “aliphatic”, as used herein, means straight-chain, branched or cyclic C₁-C₁₂ hydrocarbons which are completely saturated or which contain one or more units of unsaturation, but which are not aromatic. For example, suitable aliphatic groups include substituted or unsubstituted linear, branched or cyclic alkyl, alkenyl, alkynyl groups and hybrids thereof, such as cycloalkyl, (cycloalkyl)alkyl, (cycloalkenyl) alkyl or (cycloalkyl)-alkenyl. In various embodiments, the aliphatic group has one to ten, one to eight, one to six, one to four, or one, two, or three carbons.

The terms “alkyl”, “alkenyl”, and “alkynyl”, used alone or as part of a larger moiety, refer to a straight and branched chain aliphatic group having from one to twelve carbon atoms. For purposes of the present invention, the term “alkyl” will be used when the carbon atom attaching the aliphatic group lo the rest of the molecule is a saturated carbon atom. However, an alkyl group can include unsaturation at other carbon atoms. Thus, alkyl groups include, without limitation, methyl, ethyl, propyl, allyl, propargyl, butyl, pentyl, and hexyl. The term “alkoxy” refers to an —O-alkyl radical.

For purposes of the present disclosure, the term “alkenyl” will be used When the carbon atom attaching the aliphatic group to the rest of the molecule forms part of a carboncarbon double bond. Alkenyl groups include, without limitation, vinyl, 1-propenyl, 1-butenyl, 1-pentenyl, and 1-hexenyl.

For purposes of the present disclosure, the term “alkynyl” will be used when the carbon atom attaching the aliphatic group to the rest of the molecule forms part of a carbon-carbon triple bond. Alkynyl groups include, without limitation, ethynyl, 1-propynyl, 1-butynyl, 1-pentynyl, and 1-hexynyl.

The term “cycloaliphatic”, used alone or as part of a larger moiety, refers to a saturated or partially unsaturated cyclic aliphatic ring system having from 3 to about 14 members, wherein the aliphatic ring system is optionally substituted. In some embodiments, the cycloaliphatic is a monocyclic hydrocarbon having 3-8 or 3-6 ring carbon atoms. Non-limiting examples include cyclopropyl, cyclobutyl, cycloopentyl, cyclopentenyl, cyclohexyl, cyclohexenyl, cycloheptyl cyclohephenyl, cyclooctyl, cyclooctenyl, and cyclooctadienyl. In some embodiments, the cycloaliphatic is a bridged or fused bicyclic hydrocarbon having 6-12, 6-10, or 6-8 ring carbon atoms, wherein any individual ring in the bicyclic ring system has 3-8-members.

In some embodiments, two adjacent substituents on a cycloaliphatic ring, taken together with the intervening ring atoms, than an optionally substituted fused 5- to 6-membered aromatic or 3- to 8-membered non-aromatic ring having 0-3 ring heteroatoms selected from the group consisting of O, N, and S. Thus, the term “cycloaliphatic” includes aliphatic rings that are fused to one or more aryl, heteroaryl, or heterocyclyl rings, where the radical or point of attachment is on the aliphatic ring. Nonlimiting examples include indanyl, 5,6,7,8-tetrahydroquinoxalinyl, decahydronaphthyl, or tetrahydronaphthyl, where the radical orpoint of attachment is on the aliphatic ring.

The terms “haloaliphatic”, “haloalkyl”, “haloalkenyl” and “haloalkoxy” refer to an aliphatic, alkyl, alkenyl or alkoxy group, as the case can be, which is substituted with one or more halogen atoms. As used herein, the term “halogen” or “halo” means F, Cl, Br, or I. The term “fluoroaliphatic” refers to a haloaliphatic wherein the halogen is fluoro. Nonlimiting examples of fluoroaliphatics include —CH₂F, —CHF₂, —CH₃, —CH₂CH_{3y}, —CH₂CH₃, and —CH₂CH₃.

The terms “aryl” and “ar-”, used along or as part of a larger moiety, e.g., “aralkyl”, “aralkoxy”, or “aryloxyalkyl”, refer to a C₆ to C₁₄ aromatic hydrocarbon, comprising one to three rings, each of which is optionally substituted. Preferably, the aryl group is aryl group. Aryl groups include, without limitation, phenyl, naphthyl, and antllracenyl. In some embodiments, two adjacent substituents on an aryl ring, taken together with the intervening ring atoms, form an optionally substituted fused 5- to 6-membered aromatic or 4- to 8-membered non-aromatic ring having 0-3 ring heteroatoms selected from the group consisting of O, N, and S. thus, the term “aryl”, as used herein, includes groups in which an aromatic ring is fused to one or more heteroaryl, cycloaliphatic, or heterocyclyl rings, where the radical or point of attachment is on the aromatic ring. Nonlimiting examples of such fused ring systems include inolyl, isoindolyl, benzosothienyl, benzofuranyl, dibenzofuranyl, indazolyl, benzimidazolyl, benzthiazolyl, quinolyl, isoquinolyl, quinolinyl, phthalazinyl, quinazolinyl, quinoxalinyl, carbazolyl, acridinyl, phenazinyl, phenothiazinyl, phenoxazinyl, tetrahydroquinolinyl, tetrahydroisoquinolinyl, fluorenyl, indanyl, phenanthridinyl, tetrahydronaphthyl, indolinyl, phenoxazinyl, benzodioxanyl, and benzodioxolyl. An aryl group can be mono-, bi-, tri-, or polycyclic, preferably mono-, bi-, or tricyclic, more preferably mono- or bicyclic. The term “aryl” can be used interchangeably with the terms “aryl group”, “aryl moiety”, and “aryl ring.”

An “aralkyl” or “arylalky” group comprises an aryl group covalently attached to an alkyl group, either of which independently is optionally substituted. Preferably, the aralkyl group is C₆₋₁₀ aryl(C₆₋₁₀)alkyl, including, without limitation, benzyl, phenethyl, and naphthylmethyl.

The terms “heteroaryl” and “heteroar-”, used alone or as part of a larger moiety, e.g., heteroaralkyl, or “heteroaralkoxy”, refer to groups having 5 to 14 ring atoms, preferably 5, 6, 9, or 10 ring atoms; having 6, 10, or 14π electrons shared in a cyclic array; and having, in addition to carbon atoms, from one to four heteroatoms. The term “heteroatom” refers to nitrogen, oxygen, or sulfur, and includes any oxidized form of nitrogen or sulfur, and any quaternized form of a basic nitrogen. Thus, when used in reference to a ring atom of a heteroaryl, the term “nitrogen” includes an oxidized nitrogen (as in pyridine N-oxide). Certain nitrogen atoms of 5-membered heteroaryl groups also are substitutabte, as, further defined below. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl.

In some embodiments, two adjacent substituents on a heteroaryl ring, taken together with the intervening ring atoms, form an optionally substituted fused 5- to 6-membered aromatic or 4- to 8-membered non-aromatic ring having 0-3 ring heteroatoms selected from the group consisting of O, N, and S. Thus, the terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzotllienyl, benzofuranyl, dibenzoferanyl, indazolyl, benzimidazolyl, benzthiazolyl, quinolyl, isoquinolyl, acridinyl, phenazinyl, quinazolinyl, quinoxalinyl, 4H-quinolizinyl, carbaxolyl, acridinyl, phenazinyl, phenothiazinyl, phenoxazinyl, tetrahydroquinolinyl, tetrahydroisoquinoilinyl, and pyrido[2,3-b]-1,4-oxazin-3 (4H)-one. A heteroaryl group can be mono-, bi-, tri, or polycyclic, preferably mono-, bi-, or tricyclic, more preferably mono- or bicyclic. The term “heteroaryl” can be used interchangeably with the terms “heteroaryl ring”, or “heteroaryl group”, any of which terms include rings that are optionally substituted. The term “heteroaralkyl” refers to an alkyl group substituted by a heteroaryl, wherein the alkyl and heteroaryl portions independently are optionally substituted.

As used herein, the terms “aromatic ring” and “aromatic ring system” refer to an optionally substituted mono-, bi-, or tricyclic group having 0-6, preferably 0-4 ring heteroatoms, and having 6, 10, or 14π electrons shared in a cyclic array. Thus, the terms “aromatic ring” and “aromatic ring system” encompass both aryl and heteroaryl groups.

As used herein, the terms “heterocycle”, “heterocyclyl”, “heterocyclic radical”, and “heterocyclic ring” are used interchangeably and refer to a stable 3- to 7-membered monocyclic, or to a fused 7- to 10-membered or bridged 6- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a heterocyclyl ring having 1-3 heteroatoms selected from oxygen, sulfur or nitrogen, the nitrogen can be N (as in 3,4-dihydro-2H-pyrrolyl), NH (as in pyrrolidinyl), or *NR (as in N-substituted pyrrolidinyl). A heterocyclic ring can be attached to its pendant group at any heteroatom or carbon atom that results in a stable structure, and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydrothienyl, decahydroquinolinyl, oxaxolidinyl, piperazinyl, dioxanyl, dioxolanyl, diazepinyl, oxazepinyl, thiazepinyl, morpholinyl, and quinuelidinyl.

In some embodiments, two adjacent substituents on a heterocyclic ring, taken together with the intervening ring atoms, form an optionally substituted fused 5 to 6-membered aromatic or 3- to 8-membered non-aromatic ring having 0-3 ring heteroatoms selected from the group consisting of O, N, and S. Thus, the terms “heterocycle”, “heterocyclyl”, “heterocyclyl ring”, “heterocyclic group”, “heterocyclic moiety”, and “heterocyclic radical”, are used interchangeably herein, and include groups in which a heterocyclyl ring is fused to one or more aryl, heteroryl, or cycloaliphatic rings, such as indolinyl, 3H-indolyl, cllromanyl, phenanthridinyl, or tetrahydroquinolinyl, where the radical or point of attachment is on the heterocyclyl ring. A heterocyclyl group can be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond between ring atoms. The term “partially unsaturated” is intended to encompass rings having multiple sites of unsaturation, but is not intended to include aryl or heteroaryl moieties, as herein defined.

The term “linker group” or “linker” means an organic moiety that connects two parts of a compound. Linkers typically comprise an atom such as ocygen or sulfur, a unit such as NH, CH2, C(O), C(O)NH, or a chain or atoms, such as an alkylene chain. The molecular mass of a linker is typically in the range of about 14 to 200, preferably in the range of 14 to 96 with a length of up to about six atoms. In some embodiments, the linker is a C1_{—}6 alkylene chain which is optionally substituted.

The term “alkylene” refers to a bivalent alkyl group, an “alkylene chain” is a polymethylene group, i.e., —(DCH₄)ₙ—, wherein n is a positive integer, preferably from one to six, from one to four, from one to three, from one to two, or from two to three. A substituted alkylene chain is a polymethylene group in which one or more methylene hyddrogen atoms is replaced with a substituent. Suitable substituents include those described below for a substituted aliphatic group. An alkylene chain also can be substituted at one or more positions with an aliphatic group or a substituted aliphatic group.

An alkylene chain also can be optionally interrupted by a functional group. An alkylene chain is “interrupted” by a functional group when an internal methylene unit is replaced with the functional group. Examples of suitable “interrupting functional groups” include —C(R*)═C(R*)—, —C═C—, —O—, —S—S, —S(O)—, —S(O)₂—, —S(O)₂N(R⁺)—, —N(R⁺)—, —N(R⁺)CO—, N(R⁺)CO₂—, —N(R⁺)C(O)N(R⁺)—, —C(O)N(R⁺)—, —C(O)—, —C(O)—C(O)—, —CO₂—, —OCC(O)—, —OC(O)N(R⁺)—, or N(R⁺)S(O)₂. Each R⁺, independently, is hydrogen or an optionally substituted aliphatic, aryl, heteroaryl, or heterocyclyl group, or two R⁺ on the same nitrogen atom, taken together with the nitrogen atom, form a five to eight membered aromatic or non-aromatic ring having, in addition to the nitrogen atom, zero to two ring heteroatoms selected from N, O, and S. Each R* independently is hydrogen or an optionally substituted aliphatic, aryl, heteroaryl, or heterocyclyl group.

One of ordinary skill in the art will recognize that when an alkylene chain having an interruption is attached to a functional group, certain combinations are not sufficiently stable for pharmaceutical use. Only stable or chemically feasible compounds are within the scope of the present invention. A stable or chemically feasible compound is one in which the chemical structure is not substantially altered when kept at a temperature from about −80° C. to about +40° C., preferably from about −20° C. to about +40° C., in the absence of moisture or other chemically reactive conditions, for at least a week, or a compound which maintains its integrity long enough to be useful for therapeutic or prophylactic administration to a patient.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group can have a substituent at each substitutable position of the group, and the substituents can be either the same or different. As used herein, the term “independently selected” means that the same or different values can be selected for multiple instances of a given variable in a single compound.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a 13C- or 14C-enriched carbon are within the scope of the invention.

It also will be apparent to one skilled in the art that certain compounds of this invention can exist in tautomeric forms, all such tautomeric forms of the compounds being within the scope of the invention. Unless stereochemical configuration is expressly defined, structures depicted herein are meant to include all stereochemical forms of the structure; i.e., the R and S configurations for each asymmetric center. Therefore, unless otherwise indicated, single stereochemical isomers as well as enantiomeric and diastereomeric mixtures of the present compounds are within the scope of the invention. By way of example, the compounds of formula (I) wherein R^{a} is hydroxy can have R or S configuration at the carbon atom bearing R^{a}. Both the R and the S stereochemical isomers, as well as all mixtures thereof are included within the scope of the invention.

Where stereochemical configuration at a given asymmetric center is defined by structure, unless stated otherwise, the depicted configuration indicates stereochemistry relative to other asymmetric centers in the molecule. Where stereochemical configuration is defined by chemical name, the designations (rel), (R⁺), and (S⁺) indicate relative stereochemistry, while the designations (+), (−), (R), (S), and (abs) indicate absolute stereochemistry.

In the compounds of formula (I), where relative stereochemistry is defined, the diastereomeric purity of the compound preferably is at least 80%, more preferably at least 90%, still more preferably at least 95%, and most preferably at least 99%. As used herein, the term, “diastereometric purity” refers to the amount of a compound having the depicted relative stereochemistry, expressed as a percentage of the total amount of all diastereomers present.

In some embodiments, stereochemical configurations depicted at asterisked positions indicate absolute as well as relative stereochemistry. Preferably, the enantiomeric purity of the compound is at least 80%, more preferably at least 90%, still more preferably at least 95%, and most preferably at least 99%. As used herein, the term “enantiomeric purity” refers to the amount of a compound having the depicted absolute stereochemistry, expressed as a percentage of the total amount of the depicted compound and its enantiomer.

Methods for determining diastereomeric and enantiomeric purity are well-known in the art. Diastereomeric purity can be determined by any analytical method capable of quantitatively distinguishing between a compound and its diastereomers. Examples of suitable analytical methods include, without limitation, unclear magnetic resonance spectroscopy (NMR), gas chomatography (GC), and high performance liquid chromatography (HPLC). Similarly, enantiomeric purity can be determined by any analytical method capable of quantitatively distinguishing between a compound and its enantiomer. Examples of suitable analytical methods include, without limitation, GC or HPLC, using a chiral column packing material. Enantiomers can also be distinguishable by NMR if first derivatized with an optically enriched derivatizing agent, e.g., Mosher's acid.

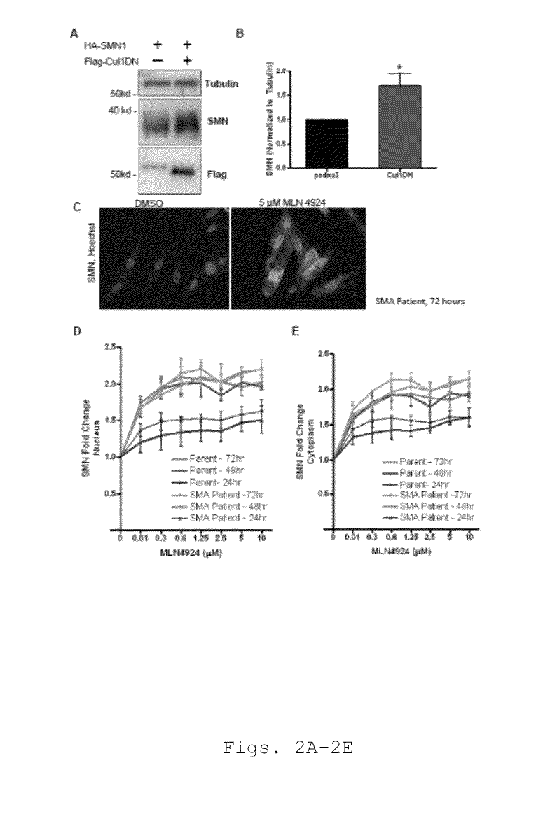

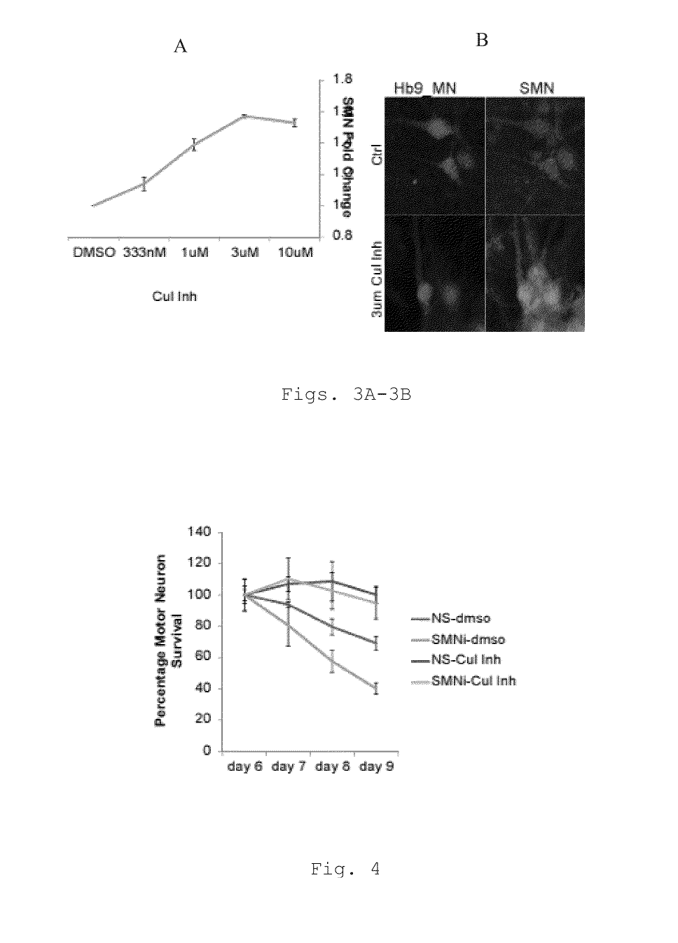

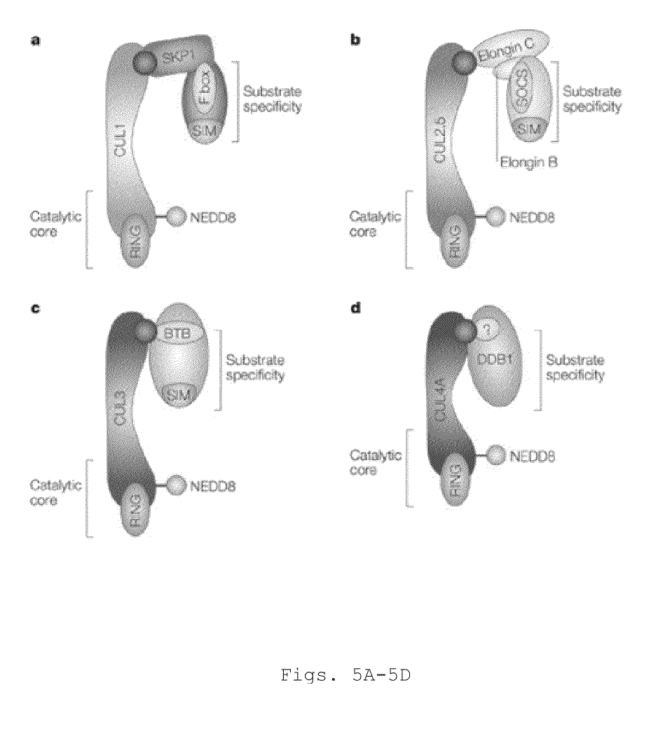

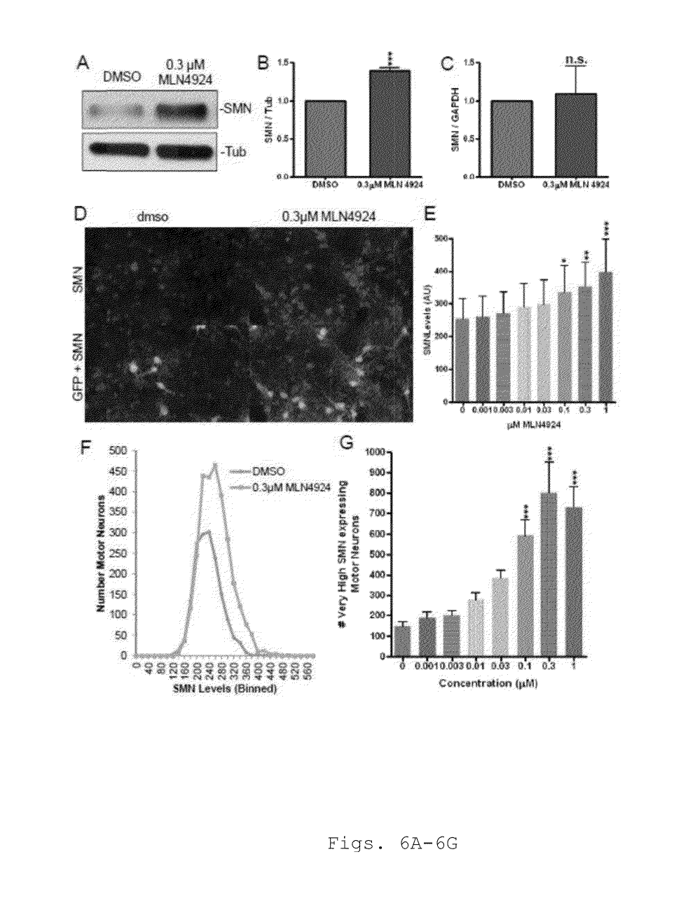

As used herein, the term “high SMN expressor” refers to a neuron (e.g., motor neuron) that displays SMN levels (e.g., protein) greater than the top 10^{th} percentile as defined in the DMSO control (see the experimental results accompanying FIGS. 2A-2G).

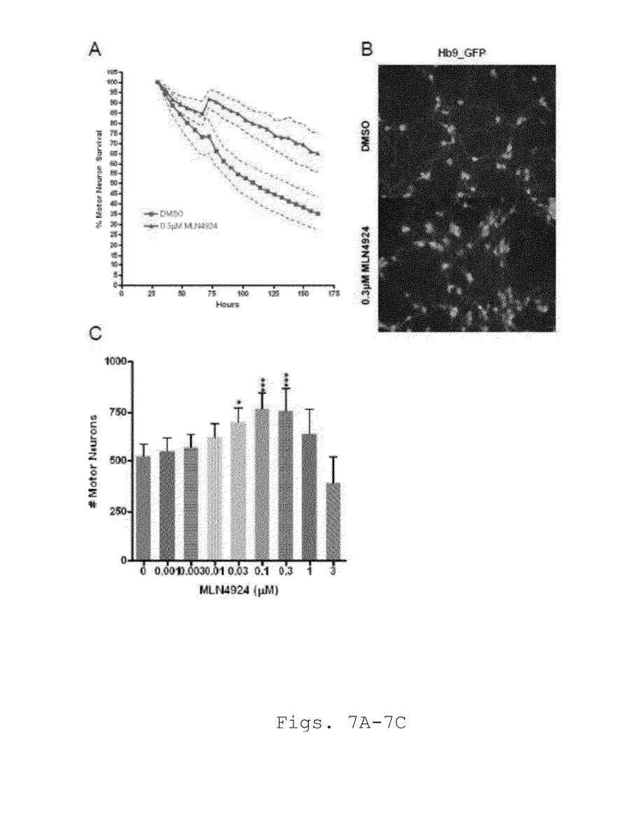

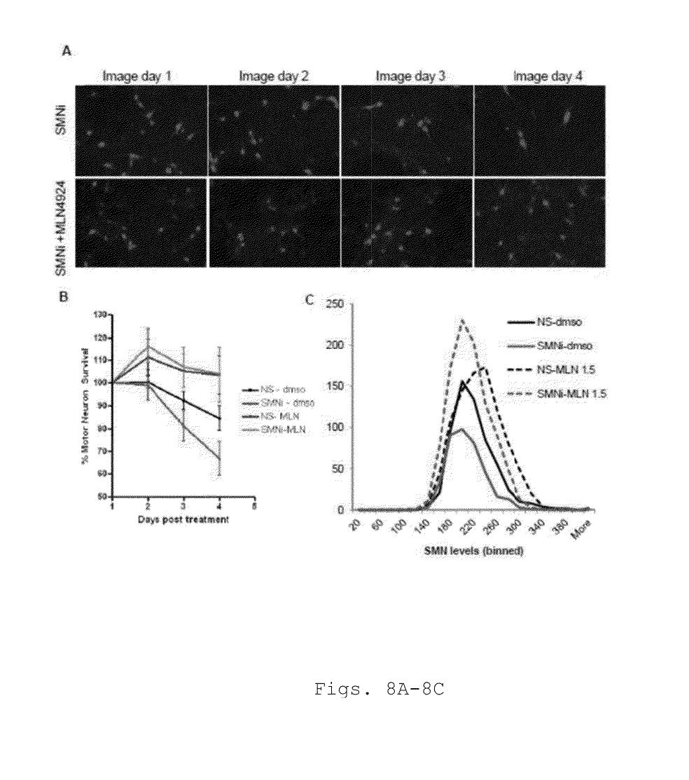

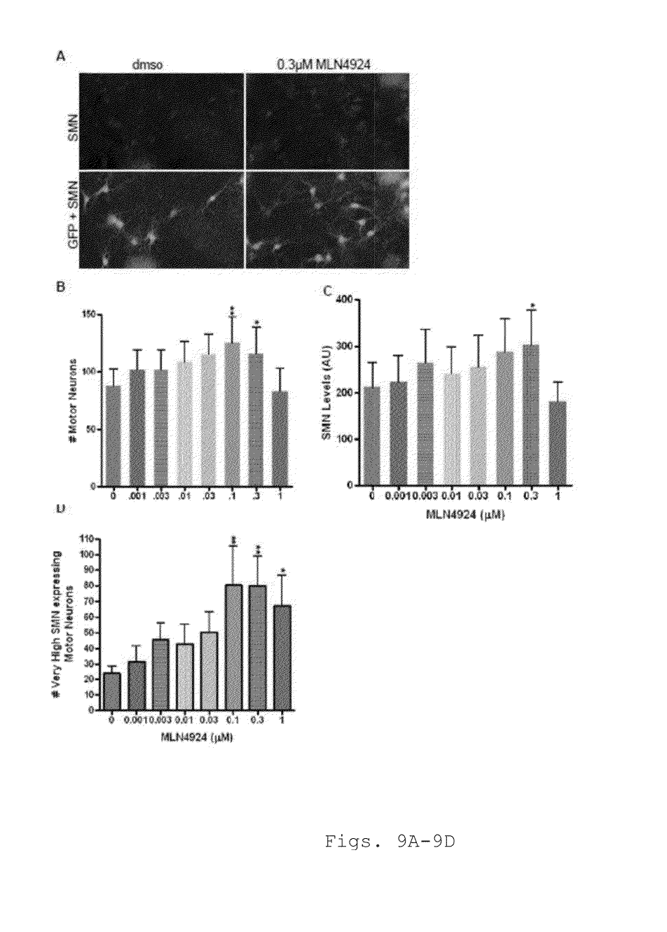

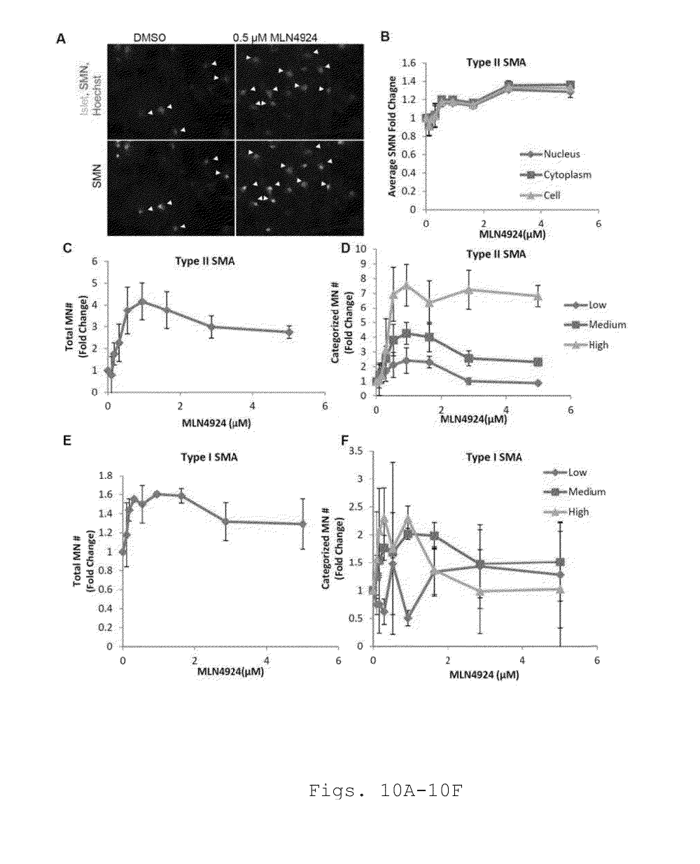

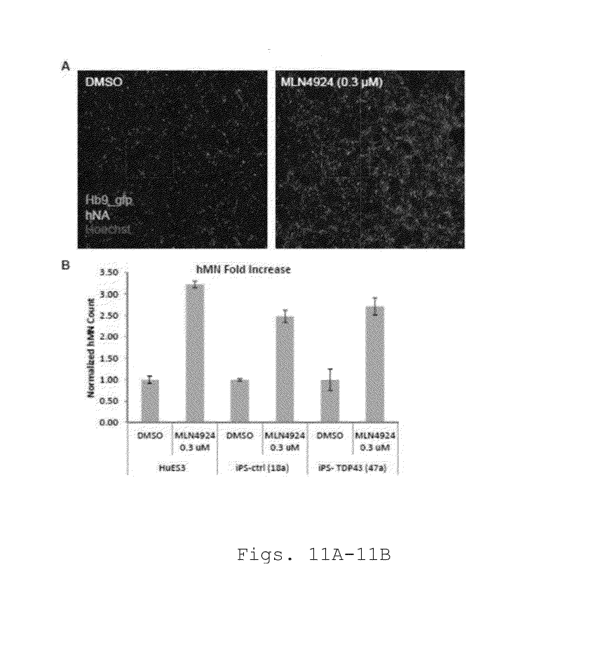

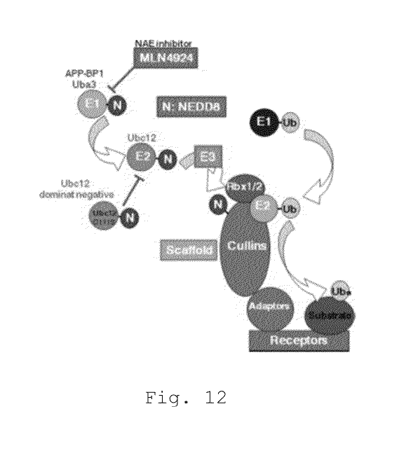

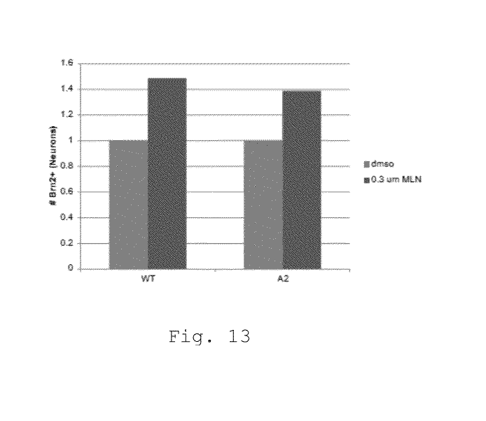

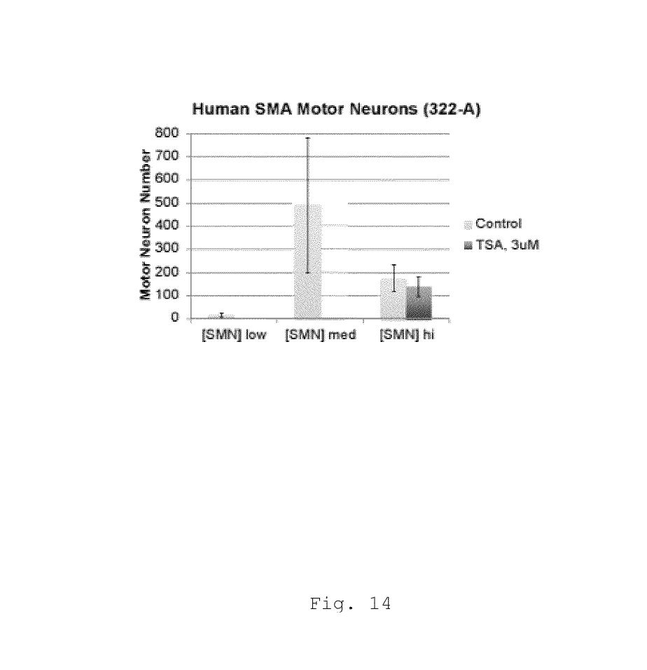

As used herein, the term “average SMN expressor” refers to a neuron (e.g., motor neuron) that displays SMN levels (e.g., protein) lower than the top 10^{th} percentile and higher than the bottom ˜10^{th} percentile as defined in the DMSO control (see the experimental results accompanying FIGS. 6A-2G).

As used herein, the term “low SMN expressor” refers to a neuron (e.g., motor neuron) that displays SMN levels (e.g., protein) lower than the bottom ˜10^{th} percentile as defined in the DMSO control (see the experimental results accompanying FIGS. 6A-2G).

As used herein, the term “SMN-deficient” in the context of a neuron (e.g., motor neuron, e.g., human motor neuron) refers to a neuron in which SMN protein is dysfunctional or degraded or the levels of SMN protein in the neuron are decreased relative to a control or reference level of SMN protein in a normal or healthy neuron.

What is claimed is:

1. A method of regulating survival of motor neuron (SMN) protein levels in a cell, comprising contacting the cell with an effective amount of an agent which inhibits the level or activity of Nedd8 activating enzyme (NAE) in the cell, thereby regulating SMN protein levels in the cell.

2. A method according to claim 1, wherein regulating SMN protein levels in the cell is selected from the group consisting of (i) increasing the levels of SMN protein in the cell, (ii) stabilizing levels of SMN protein in the cell, (iii) reducing degradation of SMN protein in the cell, (iv) preventing degradation of SMN protein in the cell, and (v) any combination of (i)-(iv).

3. A method according to claim 1, wherein the agent is a compound of formula (I): 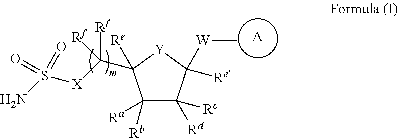
wherein
Ring A is a 6-membered nitrogen-containing heteroaryl ring, optionally fused to a 5- or 6-membered aryl, heteroaryl, cycloaliphatic or heterocyclic ring, wherein either or both rings is optionally substituted and one ring nitrogen atom is optionally osidized or Ring A is selected from the group consisting of 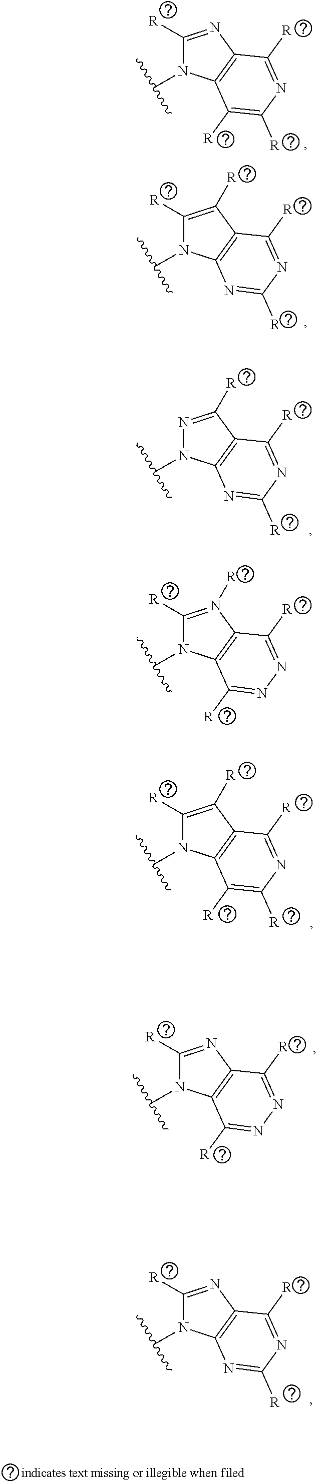
W is CH₂, CHF, CH₂, CH(R1), CH(R¹), NH, N(R), O, S, or —NHC(O)—;
R¹ is C₁₋₄ aliphatic or C₁₋₄ fluoroaliphatic; or R¹ is a C₂₋₄ alkylene chain that is attached to a ring position on Ring A to form a 5-, 6-, or 7-membered fused ring, wherein the alkylene chain optionally is substituted with C₁₋₄ aliphatic, C₁₋₄ fluoroaliphatic, —O, —CN, or —C(O)N(R⁴)2;
X is —O—, —S—, or —C(R^{m})(Rⁿ)—;
Y is —O—, —S—, or —C(R^{m})(Rⁿ)—;
R^{a} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)NR⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, or —N(R^{4x})(R^{4y}); —CO₂R⁵, or —C(O)N(R^{4x})(R4^{4y}); or R^{a} and R^{c} together form a bond;
R^{b} is selected from the group consisting of hydrogen, fluoro, C₁₋₄ aliphatic, and C₁₋₄ fluoroaliphatic;
R^{c} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;
R^{d} is selected from the group consisting of hydrogen, fluoro, C₁₋₄ aliphatic, and C₁₋₄ fluoroaliphatic;
R^{e} is hydrogen, or C₁₋₄ aliphatic; or R^{e}, taken together with one R^{f} and the intervening carbon atoms, forms a 3- to 6-membered spirocyclic ring; or R^{e}, taken together with R^{m} and the intervening carbon atoms, forms a fused cyclopropane ring, which is optionally substituted with one or two substituents independently selected from fluoro or C₁₋₄ aliphatic;
R^{e′} is hydrogen or C₁₋₄ aliphatic; or R^{e′}, taken together with R^{m} and the intervening carbon atoms, forms a fused cyclopropane ring, which is optionally substituted with one or two substituents independently selected from fluoro or C₁₋₄ aliphatic;
each R^{f} is independently hydrogen, fluoro, C₁₋₄ aliphatic, or C₁₋₄ fluoroaliphatic; or two R^{f} taken together form ═O; or two R^{f}, taken together with the carbon atom to which they are attached, form a 3- to 6-membered carbocyclic ring; or one R^{f}, taken together with R^{e} and the intervening carbon atoms, forms a 3- to 6-membered spirocyclic ring;
R^{m} is hydrogen, fluoro, —N(R⁴)₂, or an optionally substituted C₁₋₄ aliphatic group; or R^{m} and Rⁿ together form ═O or ═C(R⁵)₂; or R^{m} and R^{e}, taken together with the intervening carbon atoms, form a fused cyclopropane ring, which is optionally substituted with one or two substituents independently selected from fluoro or C₁₋₄ aliphatic; or R^{m} and R^{e′}, taken together with the intervening carbon atoms, form a fused cyclopropane ring, which is optionally substituted with one or two substituents independently selected from fluoro or C₁₋₄ aliphatic;
Rⁿ is hydrogen, fluoro, or an optionally substituted C₁₋₄ aliphatic group; or R^{m} and Rⁿ together form ═O or ═C(R⁵)₂;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, in addition to the nitrogen atom, 0-2 ring heteroatoms independently selected from N, O, and S;
R^{4x} is hydrogen, C₁₋₄ alkyl, C₁₋₄ fluoroalkyl, or C₆₋₁₀ar(C₁₋₄)alkyl, the aryl portion of which can be optionally substituted;
R^{4y} is hydrogen, C₁₋₄ alkyl, C₁₋₄ fluoroalkyl, C₆₋₁₀ar(C₁₋₄)alkyl, the aryl portion of which can be optionally substituted, or an optionally substituted 5- or 6-membered aryl, heteroaryl, or heterocyclyl ring; or
R^{4x} and R^{4y}, taken together with the nitrogen atom to which they are attached, form an optionally substituted 4- to 5-membered heterocyclyl ring having, in addition to the nitrogen atom, 0-2 ring heteroatoms independently selected from N, O, and S; and
each R⁵ independently is hydrogen or an optionally substituted aliphatic, aryl, heteroaryl, or heterocyclyl group;
each R^{5x} independently is hydrogen, C₁₋₄ alkyl, C₁₋₄ fluoroalkyl, or an optionally substituted C₆₋₁₀ aryl or C₆₋₁₀ar(C₁₋₄)alkyl;
each R⁶ independently is an optionally substituted aliphatic, aryl or heteroaryl group; R^{g} is hydrogen, halo, —NO₂, —CN, —C(R⁵)═C(R⁵)₂, —C═C—R, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OCO₂R⁶, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —N(R⁴)—N(R⁴)_{2l , —N(R}⁴)—OR⁵, ——C(═NR⁴)—N(R⁴)—OR⁵, —C(R⁶)═N—OR⁵, or an optionally substituted aliphatic, aryl, heteroaryl, or heterocyclyl;
each R^{h} independently is hydrogen, halo, —CN—, —OR⁵, —N(R⁴)², —SR⁶, or an optionally substituted C1-4 aliphatic group;
each R^{j} is hydrogen, —OR⁵, —SR⁶, —N(R⁴)₂, or an optionally substituted aliphatic, aryl or heteroaryl group;
R^{k} is hydrogen, halo, —OR⁵, —SR⁶, —N(R⁴)+, or an optionally substituted C₁₋₄ aliphatic group;
m is 1, 2, or 3;
and pharmaceutically acceptable salts thereof.

4. A method according to claim 1, wherein the agent comprises the compound ((1S,2S,4R)-4-(4(((S)-2,3-dihydro-1H-inden-1-yl)amino)-7H-pyrrolo[2,3-d]pyrimidin-7-yl)-2-hydroxycyclopentyl)methyl sulfamate (MLN4924).

5. A method according to claim 1, further comprising contacting the cell with an effective amount of an agent which increases the level of SMN protein selected from the group consisting of a PI3 kinase inhibitor, an RTK ligand, a GSK3 kinase inhibitor, and combinations thereof.

6. A method according to claim 1, wherein the cell comprises a neuron or a motor neuron.

7. A method according to claim 1, wherein contacting the cell occurs ex vivo or in vitro and the cell is obtained from a human or animal subject selected for treatment of a neurodegenerative disorder-selected from the group consisting of: (i) a neurodegenerative disorder characterized by degeneration of motor neurons; (ii) a neurodegenerative disorder characterized by diminished levels of SMN protein; (iii) a neurodegenerative disorder characterized by degradation of SMN protein; or iv) a neurodegenerative disorder is selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telangiectasia; Batten disease; Canavan disease; Cockayne syndrome; corticobasal degeneration; Creutzfeldt-Jakob disease; ischemia stroke; Krabbe disease; Lewy body dementia; multiple sclerosis; multiple system atrophy; Parkinson's disease; Pelizaeus-Merzbacher disease; Pick's disease; primary lateral sclerosis; Refsum's disease; Sandhoff disease; Schilder's disease; spinal cord injury; spinal muscular atrophy (SMA); Steele Richardson-Olszewski disease; and Tabes dorsalis.

8. A method according to claim 1, wherein contacting the cell occurs in vivo in a subject selected for treatment of a neurodegenerative disorder is selected from the group consisting of: (i) a neurodegenerative disorder characterized by degeneration of motor neurons; (ii) a neurodegenerative disorder characterized by diminished levels of SMN protein; (iii) neurodegenerative disorder characterized by degradation of SMN protein; or iv) a neurodegenerative disorder selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telangiectasia; Batten disease; Canavan disease; Cockayne syndrome; corticobasal degeneration; Creutzfeldt-Jakob disease; ischemia stroke; Krabbe disease; Lewy body dementia; multiple sclerosis; multiple system atrophy; Parkinson's disease; Pelizaeus-Merzbacher disease; Pick's disease; primary lateral sclerosis; Refsum's disease; Sandhoff disease; Schilder's disease; spinal cord injury; spinal muscular atrophy (SMA); Steele Richardson-Olszewski disease; and Tabes dorsalis.

9. A method of promoting sell survival in a subject in need thereof by regulating the overall levels of SMN protein in tits subject comprising administering to a subject an agent which inhibits the level or activity of Nedd8 activating enzyme (NAE) in the subject's cells, thereby promoting cell survival in the subject.

10. A method according to claim 9, wherein regulating the overall levels of SMN protein in the subject is selected from the group consisting of: (i) increasing the levels of SMN protein in the subject's cells, (ii) stabilizing levels of SMN protein in the subject's cells, (iii) reducing degradation of SMN protein in the subject's cells, (iv) preventing degradation of SMN protein in the subject's cells, (v) increasing the number of high SMN expressing cells in the subject, (vi) stabilizing the number of high SMN expressing cells in the subject, (vii) increasing the ratio of high SMN expressing cells to low SMN expressing cells in the subject, and (viii) decreasing the number of low SMN expressing cells in the subject, and (ix) any combination of (i)-(viii).

11. A method according claim 9, wherein the agent is a compound of formula (I): 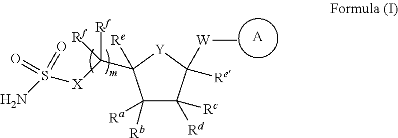
wherein:
Ring A is a 6-membered nitrogen-containing heteroaryl ring, optionally fused to a 5- or 6-membered aryl, heteroaryl, cycloaliphatic or heterocyclic ring, wherein either or both rings is optionally substituted and one ring nitrogen atom is optionally oxidized or Ring A is selected from the group consisting of 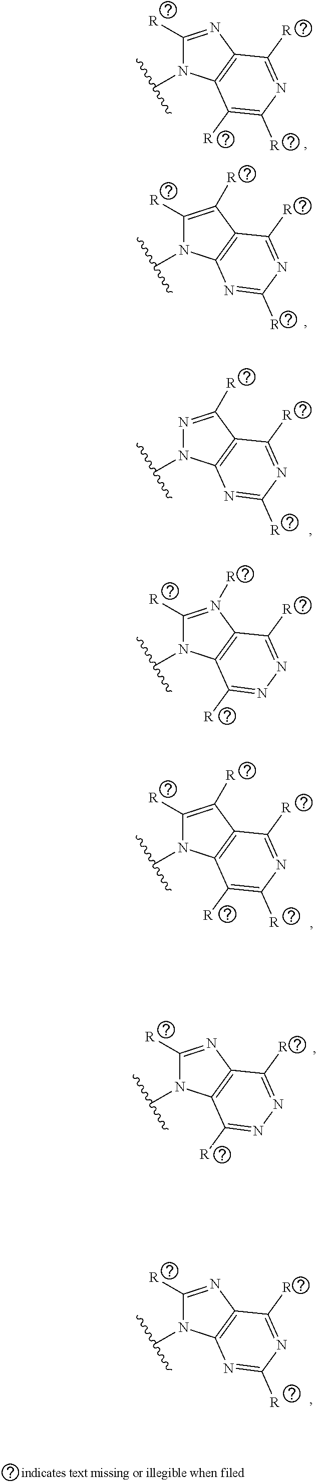
W is CH₂, CHF, CH₂, CH(R1), CH(R¹), NH, N(R), O, S, or —NHC(O)—;
R¹ is C₁₋₄ aliphatic or C₁₋₄ fluoroaliphatic; or R¹ is a C₂₋₄ alkylene chain that is attached to a ring position on Ring A to form a 5-, 6-, or 7-membered fused ring, wherein the alkylene chain optionally is substituted with C₁₋₄ aliphatic, C₁₋₄ fluoroaliphatic, —O, —CN, or —C(O)N(R⁴)2;
X is CH2, CHF, CH2, NH, or O;
Y is —O—, —S—, or —C(R^{m})(Rⁿ)—;
R^{a} is selected from the group consisting of hydrogen, gluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{6y}); or R^{a} and R^{c} together form a bond;
R^{b} is selected from the group consisting of hydrogen, fluoro, C₁₋₄ aliphatic, and C₁₋₄ fluoroaliphatic;
R^{c} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{b} and R^{c} together form a bond;
R^{d} is selected from the group consisting of hydrogen, fluoro, C₁₋₄ aliphatic, and C₁₋₄ fluoroaliphatic;
R^{e} is hydrogen, or C₁₋₄ aliphatic; or R^{e}, taken together with one R^{f} and the intervening carbon atoms, forms a 3- to 6-membered spirocyclic ring; or R^{e}, taken together with R^{m} and the intervening carbon atoms, forms a fused cyclopropane ring, which is optionally substituted with one or two substituents independently selected from fluoro or C₁₋₄ aliphatic;
R^{e′} is hydrogen or C₁₋₄ aliphatic; or R^{e′}, taken together with R^{m} and the intervening carbon atoms, forms a fused cyclopropane ring, which is optionally substituted with one or two substituents independently selected from fluoro or C₁₋₄ aliphatic;
each R^{f} is independently hydrogen, fluoro, C₁₋₄ aliphatic, or C₁₋₄ fluoroaliphatic; or two R^{f} taken together form ═O; or two R^{f}, taken together with the carbon atom to which they are attached, form a 3- to 6-membered carbocyclic ring; or one R^{f}, taken together with R^{e} and the intervening carbon atoms, forms a 3- to 6-membered spirocyclic ring;
R^{m} is hydrogen, fluoro, —N(R⁴)₂, or an optionally substituted C₁₋₄ aliphatic group; or R^{m} and Rⁿ together form ═O or ═C(R⁵)₂; or R^{m} and R^{e}, taken together with the intervening carbon atoms, form a fused cyclopropane ring, which is optionally substituted with one or two substituents independently selected from fluoro or C₁₋₄ aliphatic; or R^{m} and R^{e′}, taken together with the intervening carbon atoms, form a fused cyclopropane ring, which is optionally substituted with one or two substituents independently selected from fluoro or C₁₋₄ aliphatic;
Rⁿ is hydrogen, fluoro, or an optionally substituted C₁₋₄ aliphatic group; or R^{m} and Rⁿ together form ═O or ═C(R⁵)₂;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, in addition to the nitrogen atom, 0-2 ring heteroatoms independently selected from N, O, and S;
R^{4x} is hydrogen, C₁₋₄ alkyl, C₁₋₄ fluoroalkyl, or C₆₋₁₀ar(C₁₋₄)alkyl, the aryl portion of which can be optionally substituted;
R^{4y} is hydrogen, C₁₋₄ alkyl, C₁₋₄ fluoroalkyl, C₆₋₁₀ar(C₁₋₄)alkyl, the aryl portion of which can be optionally substituted, or an optionally substituted 5- or 6-membered aryl, heteroaryl, or heterocyclyl ring; or
R^{4x} and R^{4y}, taken together with the nitrogen atom to which they are attached, form an optionally substituted 4- to 5-membered heterocyclyl ring having, in addition to the nitrogen atom, 0-2 ring heteroatoms independently selected from N, O, and S; and
each R⁵ independently is hydrogen or an optionally substituted aliphatic, aryl, heteroaryl, or heterocyclyl group;
each R^{5x} independently is hydrogen, C₁₋₄ alkyl, C₁₋₄ fluoroalkyl, or an optionally substituted C₆₋₁₀ aryl or C₆₋₁₀ar(C₁₋₄)alkyl;
each R⁶ independently is an optionally substituted aliphatic, aryl or heteroaryl group; R^{g} is hydrogen, halo, —NO₂, —CN, —C(R⁵)═C(R⁵)₂, —C═C—R, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OCO₂R⁶, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —N(R⁴)—N(R⁴)₂, —N(R⁴)—OR⁵, —C(═NR⁴)—N(R⁴)—OR⁵, —C(R⁶)═N—OR⁵, or an optionally substituted aliphatic, aryl, heteroaryl, or heterocyclyl;
each R^{h} independently is hydrogen, halo, —CN—, —OR⁵, —N(R⁴)², —SR⁶, or an optionally substituted C1-4 aliphatic group;
each R^{j} is hydrogen, —OR⁵, —SR⁶, —N(R⁴)₂, or an optionally substituted aliphatic, aryl or heteroaryl group;
R^{k} is hydrogen, halo, —OR⁵, —SR⁶, —N(R⁴)+, or an optionally substituted C₁₋₄ aliphatic group;
m is 1, 2, or 3;
and pharmaceutically acceptable salts thereof.

12. A method according to claim 9, wherein the agent comprises the compound ((1S,2S,4R)-4-(4(((S)-2,3-dihydro-1H-inden-1-yl)amino)-7H-pyrrolo[2,3-d]pyrimidin-7-yl)-2-hydroxycyclopentyl)methyl sulfamate (MLN4924).

13. A method according to claim 9, further comprising administering to the subject an effective amount of an agent which increases the level of SMN protein selected from the group consisting of a PI3 kinase inhibitor, an RTK ligand, a GSK3 kinase inhibitor, and combinations thereof.

14. A method according to claim 9, wherein the cells comprise neurons or motor neurons.

15. A method according to claim 9, wherein the subject is selected for treatment of a neurodegenerative disorder selected from the group consisting of: (i) a neurodegenerative disorder characterized by degeneration of motor neurons; (ii) a neurodegenerative disorder characterized by diminished levels of SMN protein; (iii) neurodegenerative disorder is characterized by degradation of SMN protein; or (iv) a neurodegenerative disorder selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alpers disease; Alzheimer disease; amyotrophic lateral sclerosis (ALS); ataxia telangiectasia; Batten disease; Canavan disease; Cockayne syndrome; corticobasal degeneration; Creutzfeldt-Jakob disease; ischemia stroke; Krabbe disease; Lewy body dementia; multiple sclerosis; multiple system atrophy; Parkinson's disease; Pelizaeus-Merzbacher disease; Pick's disease; primary lateral sclerosis; Refsum's disease; Sandhoff disease; Schilder's disease; spinal cord injury; spinal muscular atrophy (SMA); Steele Richardson-Olszewski disease; and Tabes dorsalis.

16. A method of treating or preventing a neurodegenerative disorder in a subject in need thereof, comprising administering to the subject at least one agent which inhibits the level or activity of Nedd8 activating enzyme (NAE).

17. A method according to claim 16, wherein the at least one agent is a compound of formula (I): 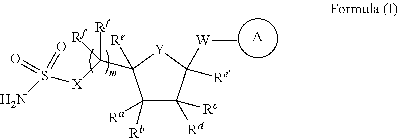
wherein
5- or 6-membered nitrogen-containing heteroaryl ring, optionally fused to a 5- or 6-membered aryl, heteroaryl, cycloaliphatic or heterocyclic ring, wherein either or both rings is optionally substituted and one ring nitrogen atom is optionally oxidized or Ring A is selected from the group consisting of 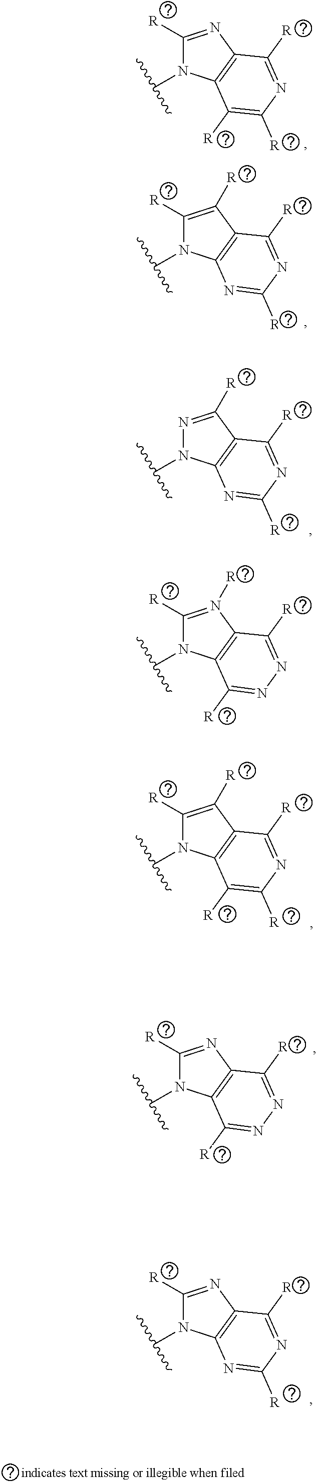
W is CH₂, CHF, CH₂, CH(R1), CH(R¹), NH, N(R), O, S, or —NHC(O)—;
R¹ is C₁₋₄ aliphatic or C₁₋₄ fluoroaliphatic; or R¹ is a C₂₋₄ alkylene chain that is attached to a ring position on Ring A to form a 5-, 6-, or 7-membered fused ring, wherein the alkylene chain optionally is substituted with C₁₋₄ aliphatic, C₁₋₄ fluoroaliphatic, —O, —CN, or —C(O)N(R⁴)2;
X is CH2, CHF, CH2, NH, or O;
Y is —O—, —S—, or —C(R^{m})(Rⁿ)—;
R^{a} is selected from the group consisting of hydrogen, gluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{6y}); or R^{a} and R^{c} together form a bond;
R^{b} is selected from the group consisting of hydrogen, fluoro, C₁₋₄ aliphatic, and C₁₋₄ fluoroaliphatic;
R^{c} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;
R^{d} is selected from the group consisting of hydrogen, fluoro, C₁₋₄ aliphatic, and C₁₋₄ fluoroaliphatic;
R^{e} is hydrogen, or C₁₋₄ aliphatic; or R^{e}, taken together with one R^{f} and the intervening carbon atoms, forms a 3- to 6-membered spirocyclic ring; or R^{e}, taken together with R^{m} and the intervening carbon atoms, forms a fused cyclopropane ring, which is optionally substituted with one or two substituents independently selected from fluoro or C₁₋₄ aliphatic;
R^{e′} is hydrogen or C₁₋₄ aliphatic; or R^{e′}, taken together with R^{m} and the intervening carbon atoms, forms a fused cyclopropane ring, which is optionally substituted with one or two substituents independently selected from fluoro or C₁₋₄ aliphatic;
each R^{f} is independently hydrogen, fluoro, C₁₋₄ aliphatic, or C₁₋₄ fluoroaliphatic; or two R^{f} taken together form ═O; or two R^{f}, taken together with the carbon atom to which they are attached, form a 3- to 6-membered carbocyclic ring; or one R^{f}, taken together with R^{e} and the intervening carbon atoms, forms a 3- to 6-membered spirocyclic ring;
R^{m} is hydrogen, fluoro, —N(R⁴)₂, or an optionally substituted C₁₋₄ aliphatic group; or R^{m} and Rⁿ together form ═O or ═C(R⁵)₂; or R^{m} and R^{e}, taken together with the intervening carbon atoms, form a fused cyclopropane ring, which is optionally substituted with one or two substituents independently selected from fluoro or C₁₋₄ aliphatic; or R^{m} and R^{e′}, taken together with the intervening carbon atoms, form a fused cyclopropane ring, which is optionally substituted with one or two substituents independently selected from fluoro or C₁₋₄ aliphatic;
Rⁿ is hydrogen, fluoro, or an optionally substituted C₁₋₄ aliphatic group; or R^{m} and Rⁿ together form ═O or ═C(R⁵)₂;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, in addition to the nitrogen atom, 0-2 ring heteroatoms independently selected from N, O, and S;
R^{4x} is hydrogen, C₁₋₄ alkyl, C₁₋₄ fluoroalkyl, or C₆₋₁₀ar(C₁₋₄)alkyl, the aryl portion of which can be optionally substituted;
R^{4y} is hydrogen, C₁₋₄ alkyl, C₁₋₄ fluoroalkyl, C₆₋₁₀ar(C₁₋₄)alkyl, the aryl portion of which can be optionally substituted, or an optionally substituted 5- or 6-membered aryl, heteroaryl, or heterocyclyl ring; or
R^{4x} and R^{4y}, taken together with the nitrogen atom to which they are attached, form an optionally substituted 4- to 5-membered heterocyclyl ring having, in addition to the nitrogen atom, 0-2 ring heteroatoms independently selected from N, O, and S; and
each R⁵ independently is hydrogen or an optionally substituted aliphatic, aryl, heteroaryl, or heterocyclyl group;
each R^{5x} independently is hydrogen, C₁₋₄ alkyl, C₁₋₄ fluoroalkyl, or an optionally substituted C₆₋₁₀ aryl or C₆₋₁₀ar(C₁₋₄)alkyl;
each R⁶ independently is an optionally substituted aliphatic, aryl or heteroaryl group; R^{g} is hydrogen, halo, —NO₂, —CN, —C(R⁵)═C(R⁵)₂, —C═C—R, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OCO₂R⁶, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —N(R⁴)—N(R⁴)₂, —N(R⁴)—OR⁵, —C(═NR⁴)—N(R⁴)—OR⁵, —C(R⁶)═N—OR⁵, or an optionally substituted aliphatic, aryl, heteroaryl, or heterocyclyl;
each R^{h} independently is hydrogen, halo, —CN—, —OR⁵, —N(R⁴)², —SR⁶, or an optionally substituted C1-4 aliphatic group;
each R^{j} is hydrogen, —OR⁵, —SR⁶, —N(R⁴)₂, or an optionally substituted aliphatic, aryl or heteroaryl group;
R^{k} is hydrogen, halo, —OR⁵, —SR⁶, —N(R⁴)+, or an optionally substituted C₁₋₄ aliphatic group;
m is 1, 2, or 3;
and pharmaceutically acceptable salts thereof.

18. A method according to claim 16, wherein the at least one agent comprises the compound ((1S,2S,4R)-4-(4(((S)-2,3-dihydro-1H-inden-1-yl)amino)-7H-pyrrolo[2,3-d]pyrimidin-7-yl)-2-hydroxycyclopentyl)methyl sulfamate (MLN4924).

19. A method according to claim 16, further comprising contacting the cell with an effective amount of an agent which increases the level of SMN protein selected from the group consisting of a PI3 kinase inhibitor, an RTK ligand, a GSK3 kinase inhibitor, and combinations thereof.

20. A method according to claim 16, wherein the neurodegenerative disorder is selected from the group consisting of: (i) a neurodegenerative disorder characterized by degeneration of motor neurons; (ii) a neurodegenerative disorder characterized by diminished levels of SMN protein; (iii) neurodegenerative disorder is characterized by degradation of SMN protein; or (iv) a neurodegenerative disorder selected from the group consisting of: a polyglutamine expansion disorders selected from the group consisting of Huntington's disease (HD), dentatorubropallidoluysian atrophy, Kennedy's disease, and spinocerebellar ataxia; a trinucleotide repeat expansion disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telangiectasia; Batten disease; Canavan disease; Cockayne syndrome; corticobasal degeneration; Creutzfeldt-Jakob disease; ischemia stroke; Krabbe disease; Lewy body dementia; multiple sclerosis; multiple system atrophy; Parkinson's disease; Pelizaeus-Merzbacher disease; Pick's disease; primary lateral sclerosis; Refsum's disease; Sandhoff disease; Schilder's disease; spinal cord injury; spinal muscular atrophy (SMA); Steele Richardson-Olszewski disease; and Tabes dorsalis.

21. A method of treating or preventing a neurodegenerative disorder in a subject in need thereof, comprising: (a) obtaining a biological sample comprising cells from a subject suspected of having a neurodegenerative disorder; (b) contacting the cells with an effective amount of at least one agent which inhibits the level or activity of Nedd8 activating enzyme (NAE) in the cells, and (c) administering the cells to the subject, thereby treating or preventing the neurodegenerative disorder.

22. A method according to claim 21, wherein the at least one agent is a compound of formula (I): 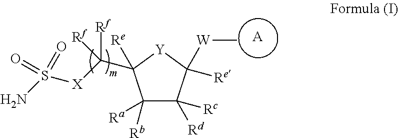
wherein:
Ring A is a 6-membered nitrogen-containing heteroaryl ring, optionally fused to a 5- or 6-membered aryl, heteroaryl, cycloaliphatic or heterocyclic ring, wherein either or both rings is optionally substituted and one ring nitrogen atom is optionally oxidized or Ring A is selected from the group consisting of 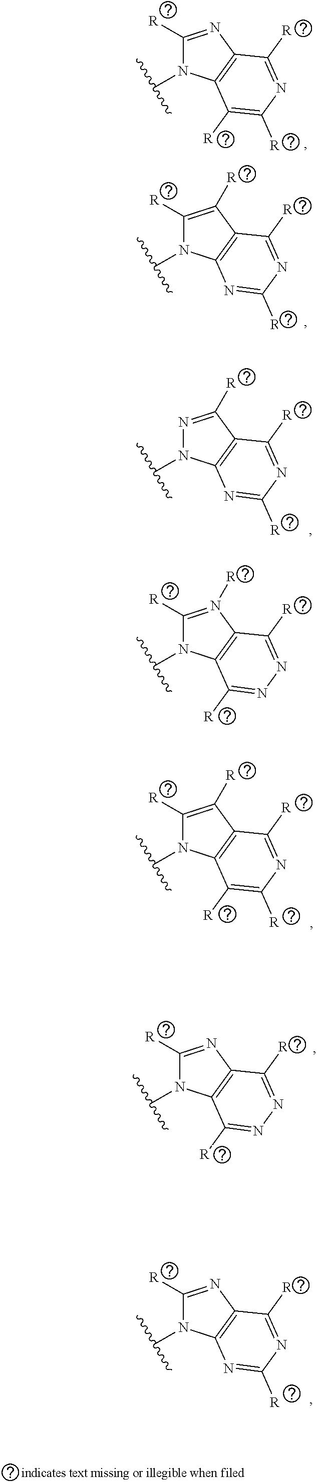
W is CH₂, CHF, CH₂, CH(R1), CH(R¹), NH, N(R), O, S, or —NHC(O)—;
R¹ is C₁₋₄ aliphatic or C₁₋₄ fluoroaliphatic; or R¹ is a C₂₋₄ alkylene chain that is attached to a ring position on Ring A to form a 5-, 6-, or 7-membered fused ring, wherein the alkylene chain optionally is substituted with C₁₋₄ aliphatic, C₁₋₄ fluoroaliphatic, —O, —CN, or —C(O)N(R⁴)2;
X is CH2, CHF, CH2, NH, or O;
Y is —O—, —S—, or —C(R^{m})(Rⁿ)—;
R^{a} is selected from the group consisting of hydrogen, gluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{6y}); or R^{a} and R^{c} together form a bond;
R^{b} is selected from the group consisting of hydrogen, fluoro, C₁₋₄ aliphatic, and C₁₋₄ fluoroaliphatic;
R^{c} is selected from the group consisting of hydrogen, fluoro, CN, N₃, OR⁵, N(R⁴)₂, NR⁴CO₂R⁶, —NR⁴C(O)R⁵, —C(O)N(R4)2, —(O)R⁵, —C(O)N(R⁴)₂, —OC(O)R⁵, —OCO₂R⁶, or a C₁₋₄ aliphatic or C₁₋₄ fluoroaliphatic optionally substituted with one or two substituents independently selected from the group consisting of —OR^{5x}, —N(R^{4x})(R^{4y}), —CO₂R^{5x}, or —C(O)N(R^{4x})(R4^{4y}); or R^{a} and R^{c} together form a bond;
R^{d} is selected from the group consisting of hydrogen, fluoro, C₁₋₄ aliphatic, and C₁₋₄ fluoroaliphatic;
R^{e} is hydrogen, or C₁₋₄ aliphatic; or R^{e}, taken together with one R^{f} and the intervening carbon atoms, forms a 3- to 6-membered spirocyclic ring; or R^{e}, taken together with R^{m} and the intervening carbon atoms, forms a fused cyclopropane ring, which is optionally substituted with one or two substituents independently selected from fluoro or C₁₋₄ aliphatic;
R^{e′} is hydrogen or C₁₋₄ aliphatic; or R^{e′}, taken together with R^{m} and the intervening carbon atoms, forms a fused cyclopropane ring, which is optionally substituted with one or two substituents independently selected from fluoro or C₁₋₄ aliphatic;
each R^{f} is independently hydrogen, fluoro, C₁₋₄ aliphatic, or C₁₋₄ fluoroaliphatic; or two R^{f} taken together form ═O; or two R^{f}, taken together with the carbon atom to which they are attached, form a 3- to 6-membered carbocyclic ring; or one R^{f}, taken together with R^{e} and the intervening carbon atoms, forms a 3- to 6-membered spirocyclic ring;
R^{m} is hydrogen, fluoro, —N(R⁴)₂, or an optionally substituted C₁₋₄ aliphatic group; or R^{m} and Rⁿ together form ═O or ═C(R⁵)₂; or R^{m} and R^{e}, taken together with the intervening carbon atoms, form a fused cyclopropane ring, which is optionally substituted with one or two substituents independently selected from fluoro or C₁₋₄ aliphatic; or R^{m} and R^{e′}, taken together with the intervening carbon atoms, form a fused cyclopropane ring, which is optionally substituted with one or two substituents independently selected from fluoro or C₁₋₄ aliphatic;
Rⁿ is hydrogen, fluoro, or an optionally substituted C₁₋₄ aliphatic group; or R^{m} and Rⁿ together form ═O or ═C(R⁵)₂;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, in addition to the nitrogen atom, 0-2 ring heteroatoms independently selected from N, O, and S;
R^{4x} is hydrogen, C₁₋₄ alkyl, C₁₋₄ fluoroalkyl, or C₆₋₁₀ar(C₁₋₄)alkyl, the aryl portion of which can be optionally substituted;
R^{4y} is hydrogen, C₁₋₄ alkyl, C₁₋₄ fluoroalkyl, C₆₋₁₀ar(C₁₋₄)alkyl, the aryl portion of which can be optionally substituted, or an optionally substituted 5- or 6-membered aryl, heteroaryl, or heterocyclyl ring; or
R^{4x} and R^{4y}, taken together with the nitrogen atom to which they are attached, form an optionally substituted 4- to 5-membered heterocyclyl ring having, in addition to the nitrogen atom, 0-2 ring heteroatoms independently selected from N, O, and S; and
each R⁵ independently is hydrogen or an optionally substituted aliphatic, aryl, heteroaryl, or heterocyclyl group;
each R^{5x} independently is hydrogen, C₁₋₄ alkyl, C₁₋₄ fluoroalkyl, or an optionally substituted C₆₋₁₀ aryl or C₆₋₁₀ar(C₁₋₄)alkyl;
each R⁶ independently is an optionally substituted aliphatic, aryl or heteroaryl group; R^{g} is hydrogen, halo, —NO₂, —CN, —C(R⁵)═C(R⁵)₂, —C═C—R, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OCO₂R⁶, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —N(R⁴)—N(R⁴)₂, —N(R⁴)—OR⁵, —C(═NR⁴)—N(R⁴)—OR⁵, —C(R⁶)═N—OR⁵, or an optionally substituted aliphatic, aryl, heteroaryl, or heterocyclyl;
each R^{h} independently is hydrogen, halo, —CN—, —OR⁵, —N(R⁴)², —SR⁶, or an optionally substituted C1-4 aliphatic group;
each R^{j} is hydrogen, —OR⁵, —SR⁶, —N(R⁴)₂, or an optionally substituted aliphatic, aryl or heteroaryl group;
R^{k} is hydrogen, halo, —OR⁵, —SR⁶, —N(R⁴)+, or an optionally substituted C₁₋₄ aliphatic group;
m is 1, 2, or 3;
and pharmaceutically acceptable salts thereof.

23. A method according to claim 21, wherein the at least one agent comprises the compound ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate (MLN4924).

24. A method according to claim 21, further comprising contacting the cells with an effective amount of an agent which increases the level of SMN protein selected from the group consisting of a PI3 kinase inhibitor, an RTK ligand, a GSK3 kinase inhibitor, and combinations thereof.

25. A method according to claim 21, wherein the neurodegenerative disorder is selected from the group consisting of: (i) a neurodegenerative disorder characterized by degeneration of motor neurons; (ii) a neurodegenerative disorder characterized by diminished levels of SMN protein; (iii) neurodegenerative disorder is characterized by degradation of SMN protein; or (iv) a neurodegenerative disorder selected from the group consisting of a polyglutamine expansion disorders selected from the group consisting of Huntington's disease (HD), dentatorubropallidoluysian atrophy, Kennedy's disease, and spinocerebellar ataxia; a trinucleotide repeat expansion, disorders selected from the group consisting of fragile X syndrome, fragile XE mental retardation, Friedreich's ataxia, myotonic dystrophy, spinocerebellar ataxia type 8, and spinocerebellar ataxia type 12; Alexander disease; Alper's disease; Alzheimer disease; amyotrophic lateral sclerosis (ALS); ataxia telangiectasia; Batten disease; Canavan disease; Cockayne syndrome; corticobasal degeneration; Creutzfeldt-Jakob disease; ischemia stroke; Krabbe disease; Lewy body dementia; multiple sclerosis; multiple system atrophy; Parkinson's disease; Pelizaeus-Merzbacher disease; Pick's disease; primary lateral sclerosis; Refsum's disease; Sandhoff disease; Schilder's disease; spinal cord injury; spinal muscular atrophy (SMA); Steele Richardson-Olszewski disease; and Tabes dorsalis.

26. A method according to claim 21, wherein the cells are selected from the group consisting of: (i) neurons (ii) motor neurons; (iii) non-neuronal cells; (iv) somatic cells; and (v) fibroblasts.

27. A method according to claim 26, wherein the cells are reprogrammed to induced pluripotent stem cells.

28. A method according to claim 26, wherein the cells are reprogrammed to neurons.

29. A method according to claim 26, wherein the cells are reprogrammed to motor neurons.

30. A method according to claim 21, wherein the cells are expanded prior to administering the cells to the subject.
